SEQUENCE LISTING (114 sequences)

SEQ ID NO: 1 - DNA, 92 bp, synthetic construct
  misc_feature 1..92: note = SPacer 1
  source 1..92: mol_type = other DNA
agctctggagcctgacaaggaggacaggagagatgctgcaagcccaagaagctctctgctcagcctgtcacaacctactgactgccagggca

SEQ ID NO: 2 - DNA, 92 bp, synthetic construct
  misc_feature 1..92: note = Spacer 2
  source 1..92: mol_type = other DNA
acgggaccgtcagtcatccaacactgtccgactcgtctctcgaagaacccgaacgtcgtagagaggacaggaggaacagtccgaggtctcga

SEQ ID NO: 3 - DNA, 30 bp, synthetic construct
  misc_feature 1..30: note = Spacer 3
  source 1..30: mol_type = other DNA
aaacgtgccaggaagctgactgccagggca

SEQ ID NO: 4 - DNA, 22 bp, synthetic construct
  misc_feature 1..22: note = misnca-02 target seq
  source 1..22: mol_type = other DNA
aaggactttcaaaggccaagga

SEQ ID NO: 5 - DNA, 22 bp, synthetic construct
  misc_feature 1..22: note = misnca-05 target seq
  source 1..22: mol_type = other DNA
tggctgagaagaccaaagagca

SEQ ID NO: 6 - DNA, 22 bp, synthetic construct
  misc_feature 1..22: note = misnca-07 target seq
  source 1..22: mol_type = other DNA
agagcaagtgacaaatgttgga

SEQ ID NO: 7 - DNA, 22 bp, synthetic construct
  misc_feature 1..22: note = misnca-12 target seq
  source 1..22: mol_type = other DNA
tggctgctgctgagaaaaccaa

SEQ ID NO: 8 - DNA, 22 bp, synthetic construct
  misc_feature 1..22: note = misnca-13 target seq
  source 1..22: mol_type = other DNA
aggaaagacaaaagagggtgtt

SEQ ID NO: 9 - DNA, 22 bp, synthetic construct
  misc_feature 1..22: note = misnca-15 target seq
  source 1..22: mol_type = other DNA
tcaaaaaggaccagttgggcaa

SEQ ID NO: 10 - DNA, 22 bp, synthetic construct
  misc_feature 1..22: note = misnca-16 target seq
  source 1..22: mol_type = other DNA
agactacgaacctgaagcctaa

SEQ ID NO: 11 - RNA, 72 nt, synthetic construct
  misc_feature 1..72: note = miR451 part
  source 1..72: mol_type = other RNA
cttgggaatggcaaggnnnnnnnnnnnnnnnnnnnnnnnnnnnnnnnnnnnnnnnctcttgctatacccaga

SEQ ID NO: 12 - RNA, 16 nt, synthetic construct
  misc_feature 1..16: note = mir451 scaffold1
  source 1..16: mol_type = other RNA
cttgggaatggcaagg

SEQ ID NO: 13 - RNA, 17 nt, synthetic construct
  misc_feature 1..17: note = mir451 scaffold2
  source 1..17: mol_type = other RNA
swcttgctatacccaga

SEQ ID NO: 14 - RNA, 22 nt, synthetic construct
  misc_feature 1..22: note = misnca-02 guide rna
  source 1..22: mol_type = other RNA
tccttggcctttgaaagtcctt

SEQ ID NO: 15 - RNA, 22 nt, synthetic construct
  misc_feature 1..22: note = miscna-05 guide rna
  source 1..22: mol_type = other RNA
tgctctttggtcttctcagcca

SEQ ID NO: 16 - RNA, 22 nt, synthetic construct
  misc_feature 1..22: note = miscna-07 guide rna
  source 1..22: mol_type = other RNA
tccaacatttgtcacttgctct

SEQ ID NO: 17 - RNA, 22 nt, synthetic construct
  misc_feature 1..22: note = miscna-12 guide rna
  source 1..22: mol_type = other RNA
ttggttttctcagcagcagcca

SEQ ID NO: 18 - RNA, 22 nt, synthetic construct
  misc_feature 1..22: note = miscna-13 guide rna
  source 1..22: mol_type = other RNA
aacaccctcttttgtctttcct

SEQ ID NO: 19 - RNA, 22 nt, synthetic construct
  misc_feature 1..22: note = miscna-15 guide rna
  source 1..22: mol_type = other RNA
ttgcccaactggtcctttttga

SEQ ID NO: 20 - RNA, 22 nt, synthetic construct
  misc_feature 1..22: note = miscna-16 guide rna
  source 1..22: mol_type = other RNA
ttaggcttcaggttcgtagtct

SEQ ID NO: 21 - RNA, 22 nt, synthetic construct
  misc_feature 1..22: note = misnca-02 second rna strand
  source 1..22: mol_type = other RNA
ccttactttcaaaggccaaggc

SEQ ID NO: 22 - RNA, 22 nt, synthetic construct
  misc_feature 1..22: note = miscna-05 second rna strand
  source 1..22: mol_type = other RNA
gccatgagaagaccaaagagcg

SEQ ID NO: 23 - RNA, 22 nt, synthetic construct
  misc_feature 1..22: note = miscna-07 second rna strand
  source 1..22: mol_type = other RNA
ctctcaagtgacaaatgttggc

SEQ ID NO: 24 - RNA, 22 nt, synthetic construct
  misc_feature 1..22: note = miscna-12 second rna strand
  source 1..22: mol_type = other RNA
gccatgctgctgagaaaaccac

SEQ ID NO: 25 - RNA, 22 nt, synthetic construct
  misc_feature 1..22: note = miscna-13 second rna strand
  source 1..22: mol_type = other RNA
tcctaagacaaaagagggtgtc

SEQ ID NO: 26 - RNA, 22 nt, synthetic construct
  misc_feature 1..22: note = miscna-15 second rna strand
  source 1..22: mol_type = other RNA
ttgaaaaggaccagttgggcac

SEQ ID NO: 27 - RNA, 22 nt, synthetic construct
  misc_feature 1..22: note = miscna-16 second rna strand
  source 1..22: mol_type = other RNA
gtcttacgaacctgaagcctac

SEQ ID NO: 28 - DNA, 86 bp, synthetic construct
  misc_feature 1..86: note = human pri-mir-144 sequence
  source 1..86: mol_type = other DNA
tggggccctggctgggatatcatcatatactgtaagtttgcgatgagacactacagtatagatgatgtactagtccgggcaccccc

SEQ ID NO: 29 - RNA, 14 nt, synthetic construct
  misc_feature 1..14: note = mir-144 scaffold 1 wherein M is an A or a C or a G or a U)
  source 1..14: mol_type = other RNA
tggggccctggctm

SEQ ID NO: 30 - RNA, 15 nt, synthetic construct
  misc_feature 1..15: note = mir-144 scaffold 2
  source 1..15: mol_type = other RNA
tttgcgatgagawmm

SEQ ID NO: 31 - RNA, 15 nt, synthetic construct
  misc_feature 1..15: note = mir-144 scaffold 3
  source 1..15: mol_type = other RNA
agtccgggcaccccc

SEQ ID NO: 32 - DNA, 28 bp, synthetic construct
  misc_feature 1..28: note = pre-144 scaffold 1
  source 1..28: mol_type = other DNA
atcggcgctatgcttcctgtgcccccag

SEQ ID NO: 33 - DNA, 92 bp, synthetic construct; identical to SEQ ID NO: 1
  misc_feature 1..92: note = pre-144 scaffold 2
  source 1..92: mol_type = other DNA

SEQ ID NO: 34 - DNA, 86 bp, synthetic construct
  misc_feature 1..86: note = miR-144 mutant helper
  source 1..86: mol_type = other DNA
tggggccctggctgggatttcatcatatactgtaagtttgcgatgagacactacagtatagatgatgtactagtccgggcaccccc

SEQ ID NO: 35 - DNA, 423 bp, synthetic construct
  misc_feature 1..423: note = SNCA140
  source 1..423: mol_type = other DNA
atggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaa

SEQ ID NO: 36 - DNA, 381 bp, synthetic construct
  misc_feature 1..381: note = SNCA126
  source 1..381: mol_type = other DNA
atggatgtattcatgaaaggactttcaaaggccaaggagggagttgtggctgctgctgagaaaaccaaacagggtgtggcagaagcagcaggaaagacaaaagagggtgttctctatgtagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaa

SEQ ID NO: 37 - DNA, 339 bp, synthetic construct
  misc_feature 1..339: note = SNCA112
  source 1..339: mol_type = other DNA
atggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaaggaagggtatcaagactacgaacctgaagcctaa

SEQ ID NO: 38 - DNA, 297 bp, synthetic construct
  misc_feature 1..297: note = SNCA98
  source 1..297: mol_type = other DNA
atggatgtattcatgaaaggactttcaaaggccaaggagggagttgtggctgctgctgagaaaaccaaacagggtgtggcagaagcagcaggaaagacaaaagagggtgttctctatgtagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaaggaagggtatcaagactacgaacctgaagcctaa

SEQ ID NO: 39 - protein, 140 aa, synthetic construct
  REGION 1..140: note = aSYN
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 40 - DNA, 10608 bp, synthetic construct
  misc_feature 1..10608: note = pVD1746
  source 1..10608: mol_type = other DNA
ttaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccatcatggagatccttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcagatctgagctccatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaagggtagttctattagcgcttactaaagcgctaatagaactaatcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatcccccgtggcatccctgtgacccctccccagtgcctctcctggccctggaagttgccactccagtgcccaccagccttgtcctaataaaattaagttgcatcagcggccgcagatctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgtttttta

SEQ ID NO: 41 - DNA, 10710 bp, synthetic construct
  misc_feature 1..10710: note = pVD1747
  source 1..10710: mol_type = other DNA
ttaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccatcatggagatccttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcagatctgagctccatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaagggtagttctattagcgcttactaaagcgctaatagaactaatcttgctatacccagaaaacgtgccaggaagctgactgccagggcacttgggaatggcaagggcgacttgtaacgtcagaggcgtctgacgttacaagtcgatcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatcccccgtggcatccctgtgacccctccccagtgcctctcctggccctggaagttgccactccagtgcccaccagccttgtcctaataaaattaagttgcatcagcggccgcagatctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgtttttta

SEQ ID NO: 42 - DNA, 10608 bp, synthetic construct
  misc_feature 1..10608: note = pVD1748
  source 1..10608: mol_type = other DNA
ttaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccatcatggagatccttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcagatctgagctccatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatcccccgtggcatccctgtgacccctccccagtgcctctcctggccctggaagttgccactccagtgcccaccagccttgtcctaataaaattaagttgcatcagcggccgcagatctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgtttttta

SEQ ID NO: 43 - DNA, 10608 bp, synthetic construct
  misc_feature 1..10608: note = pVD1749
  source 1..10608: mol_type = other DNA
ttaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccatcatggagatccttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcagatctgagctccatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggttgcccaactggtcctttttgaaaaggaccagttgggcactcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatcccccgtggcatccctgtgacccctccccagtgcctctcctggccctggaagttgccactccagtgcccaccagccttgtcctaataaaattaagttgcatcagcggccgcagatctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgtttttta

SEQ ID NO: 44 - DNA, 10608 bp, synthetic construct
  misc_feature 1..10608: note = pVD1750
  source 1..10608: mol_type = other DNA
ttaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccatcatggagatccttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcagatctgagctccatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtccttggcctttgaaagtccttactttcaaaggccaaggctcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatcccccgtggcatccctgtgacccctccccagtgcctctcctggccctggaagttgccactccagtgcccaccagccttgtcctaataaaattaagttgcatcagcggccgcagatctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgtttttta

SEQ ID NO: 45 - DNA, 10710 bp, synthetic construct
  misc_feature 1..10710: note = pVD1751
  source 1..10710: mol_type = other DNA
ttaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccatcatggagatccttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcagatctgagctccatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtccttggcctttgaaagtccttactttcaaaggccaaggctcttgctatacccagaaaacgtgccaggaagctgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatcccccgtggcatccctgtgacccctccccagtgcctctcctggccctggaagttgccactccagtgcccaccagccttgtcctaataaaattaagttgcatcagcggccgcagatctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgtttttta

SEQ ID NO: 46 - DNA, 10710 bp, synthetic construct
  misc_feature 1..10710: note = pVD1752
  source 1..10710: mol_type = other DNA
ttaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcctcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccatcatggagatccttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcagatctgagctccatggcgcgcccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgggctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagctgactgccagggcacttgggaatggcaaggttgcccaactggtcctttttgaaaaggaccagttgggcactcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatcccccgtggcatccctgtgacccctccccagtgcctctcctggccctggaagttgccactccagtgcccaccagccttgtcctaataaaattaagttgcatcagcggccgcagatctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaggtaccgactctgctgaagaggaggaaattctccttgaagtttccctggtgttcaaagtaaaggagtttgcaccagacgcacctctgttcactggtccggcgtattaaaacacgatacattgttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaataaagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctgagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcccgcacgccgataacaagccttttcatttttactacagcattgtagtggcgagacacttcgctgtcgtcgacgtacatgtatgctttgttgtcaaaaacgtcgttggcaagctttaaaatatttaaaagaacatctctgttcagcaccactgtgttgtcgtaaatgttgtttttgataatttgcgcttccgcagtatcgacacgttcaaaaaattgatgcgcatcaattttgttgttcctattattgaataaataagattgtacagattcatatctacgattcgtcatggccaccacaaatgctacgctgcaaacgctggtacaattttacgaaaactgcaaaaacgtcaaaactcggtataaaataatcaacgggcgctttggcaaaatatctattttatcgcacaagcccactagcaaattgtatttgcagaaaacaatttcggcgcacaattttaacgctgacgaaataaaagttcaccagttaatgagcgaccacccaaattttataaaaatctattttaatcacggttccatcaacaaccaagtgatcgtgatggactacattgactgtcccgatttatttgaaacactacaaattaaaggcgagctttcgtaccaacttgttagcaatattattagacagctgtgtgaagcgctcaacgatttgcacaagcacaatttcatacacaacgacataaaactcgaaaatgtcttatatttcgaagcacttgatcgcgtgtatgtttgcgattacggattgtgcaaacacgaaaactcacttagcgtgcacgacggcacgttggagtattttagtccggaaaaaattcgacacacaactatgcacgtttcgtttgactggtacgccgtcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaccgagttgtttgcgtacgtgactagcgaagaagatgtgtggaccgcagaacagatagtaaaacaaaaccctagtattggagcaataatcgatttaaccaacacgtctaaatattatgatggtgtgcattttttgcgggcgggcctgttatacaaaaaaattcaagtacctggccagactttgccgcctgaaagcatagttcaagaatttattgacacggtaaaagaatttacagaaaagtgtcccggcatgttggtgggcgtgcactgcacacacggtattaatcgcaccggttacatggtgtgcagatatttaatgcacaccctgggtattgcgccgcaggaagccatagatagattcgaaaaagccagaggtcacaaaattgaaagacaaaattacgttcaagatttattaatttaattaatattatttgcattctttaacaaatactttatcctattttcaaattgttgcgcttcttccagcgaaccaaaactatgcttcgcttgctccgtttagcttgtagccgatcagtggcgttgttccaatcgacggtaggattaggccggatattctccaccacaatgttggcaacgttgatgttacgtttatgcttttggttttccacgtacgtcttttggccggtaatagccgtaaacgtagtgccgtcgcgcgtcacgcacaacaccggatgtttgcgcttgtccgcggggtattgaaccgcgcgatccgacaaatccaccactttggcaactaaatcggtgacctgcgcgtcttttttctgcattatttcgtctttcttttgcatggtttcctggaagccggtgtacatgcggtttagatcagtcatgacgcgcgtgacctgcaaatctttggcctcgatctgcttgtccttgatggcaacgatgcgttcaataaactcttgttttttaacaagttcctcggttttttgcgccaccaccgcttgcagcgcgtttgtgtgctcggtgaatgtcgcaatcagcttagtcaccaactgtttgctctcctcctcccgttgtttgatcgcgggatcgtacttgccggtgcagagcacttgaggaattacttcttctaaaagccattcttgtaattctatggcgtaaggcaatttggacttcataatcagctgaatcacgccggatttagtaatgagcactgtatgcggctgcaaatacagcgggtcgccccttttcacgacgctgttagaggtagggcccccattttggatggtctgctcaaataacgatttgtatttattgtctacatgaacacgtatagctttatcacaaactgtatattttaaactgttagcgacgtccttggccacgaaccggacctgttggtcgcgctctagcacgtaccgcaggttgaacgtatcttctccaaatttaaattctccaattttaacgcgagccattttgatacacgtgtgtcgattttgcaacaactattgttttttaacgcaaactaaacttattgtggtaagcaataattaaatatgggggaacatgcgccgctacaacactcgtcgttatgaacgcagacggcgccggtctcggcgcaagcggctaaaacgtgttgcgcgttcaacgcggcaaacatcgcaaaagccaatagtacagttttgatttgcatattaacggcgattttttaaattatcttatttaataaatagttatgacgcctacaactccccgcccgcgttgactcgctgcacctcgagcagttcgttgacgccttcctccgtgtggccgaacacgtcgagcgggtggtcgatgaccagcggcgtgccgcacgcgacgcacaagtatctgtacaccgaatgatcgtcgggcgaaggcacgtcggcctccaagtggcaatattggcaaattcgaaaatatatacagttgggttgtttgcgcatatctatcgtggcgttgggcatgtacgtccgaacgttgatttgcatgcaagccgaaattaaatcattgcgattagtgcgattaaaacgttgtacatcctcgcttttaatcatgccgtcgattaaatcgcgcaatcgagtcaagtgatcaaagtgtggaataatgttttctttgtattcccgagtcaagcgcagcgcgtattttaacaaactagccatcttgtaagttagtttcatttaatgcaactttatccaataatatattatgtatcgcacgtcaagaattaacaatgcgcccgttgtcgcatctcaacacgactatgatagagatcaaataaagcgcgaattaaatagcttgcgacgcaacgtgcacgatctgtgcacgcgttccggcacgagctttgattgtaataagtttttacgaagcgatgacatgacccccgtagtgacaacgatcacgcccaaaagaactgccgactacaaaattaccgagtatgtcggtgacgttaaaactattaagccatccaatcgaccgttagtcgaatcaggaccgctggtgcgagaagccgcgaagtatggcgaatgcatcgtataacgtgtggagtccgctcattagagcgtcatgtttagacaagaaagctacatatttaattgatcccgatgattttattgataaattgaccctaactccatacacggtattctacaatggcggggttttggtcaaaatttccggactgcgattgtacatgctgttaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgtttttta

SEQ ID NO: 47 - DNA, 798 bp, synthetic construct
  misc_feature 1..798: note = promoter P1
  source 1..798: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 48 - DNA, 101 bp, synthetic construct
  misc_feature 1..101: note = hGH polyA sequence
  source 1..101: mol_type = other DNA
cccgtggcatccctgtgacccctccccagtgcctctcctggccctggaagttgccactccagtgcccaccagccttgtcctaataaaattaagttgcatca

SEQ ID NO: 49 - DNA, 20 bp, synthetic construct
  misc_feature 1..20: note = SNCA1 fwd primer
  source 1..20: mol_type = other DNA
aaatgttggaggagcagtgg

SEQ ID NO: 50 - DNA, 20 bp, synthetic construct
  misc_feature 1..20: note = SNCA1 rev primer
  source 1..20: mol_type = other DNA
tccagaattccttcctgtgg

SEQ ID NO: 51 - DNA, 20 bp, synthetic construct
  misc_feature 1..20: note = SNCA2 fwd primer
  source 1..20: mol_type = other DNA
atgttggaggagcagtggtg

SEQ ID NO: 52 - DNA, 20 bp, synthetic construct
  misc_feature 1..20: note = SNCA2 rev primer
  source 1..20: mol_type = other DNA
tgtcaggatccacaggcata

SEQ ID NO: 53 - DNA, 20 bp, synthetic construct
  misc_feature 1..20: note = GFP fwd
  source 1..20: mol_type = other DNA
agcaaagaccccaacgagaa

SEQ ID NO: 54 - DNA, 17 bp, synthetic construct
  misc_feature 1..17: note = GFP rev
  source 1..17: mol_type = other DNA
gcggcggtcacgaactc

SEQ ID NO: 55 - DNA, 18 bp, synthetic construct
  misc_feature 1..18: note = B2M fwd
  source 1..18: mol_type = other DNA
gccatccaccggagaatg

SEQ ID NO: 56 - DNA, 22 bp, synthetic construct
  misc_feature 1..22: note = B2M rev
  source 1..22: mol_type = other DNA
ggtggaactgagacacgtagca

SEQ ID NO: 57 - DNA, 19 bp, synthetic construct
  misc_feature 1..19: note = GAPDH fwd
  source 1..19: mol_type = other DNA
tgcccccatgtttgtgatg

SEQ ID NO: 58 - DNA, 23 bp, synthetic construct
  misc_feature 1..23: note = GAPDH rev
  source 1..23: mol_type = other DNA
gctgacaatcttgagggagttgt

SEQ ID NO: 59 - DNA, 20 bp, synthetic construct
  misc_feature 1..20: note = ACTB fwd
  source 1..20: mol_type = other DNA
agcgtggctacagcttcacc

SEQ ID NO: 60 - DNA, 24 bp, synthetic construct
  misc_feature 1..24: note = ACTB rev
  source 1..24: mol_type = other DNA
aagtctagggcaacatagcacagc

SEQ ID NO: 61 - DNA, 21 bp, synthetic construct
  misc_feature 1..21: note = HPRT fwd
  source 1..21: mol_type = other DNA
gcgaaagtggaaaagccaagt

SEQ ID NO: 62 - DNA, 23 bp, synthetic construct
  misc_feature 1..23: note = HPRT rev
  source 1..23: mol_type = other DNA
gccacatcaacaggactcttgta

SEQ ID NO: 63 - DNA, 21 bp, synthetic construct
  misc_feature 1..21: note = SNCA1 probe
  source 1..21: mol_type = other DNA
agcagggagcattgcagcagc

SEQ ID NO: 64 - DNA, 23 bp, synthetic construct
  misc_feature 1..23: note = SNCA2 probe
  source 1..23: mol_type = other DNA
cagcagccactggctttgtcaaa

SEQ ID NO: 65 - DNA, 20 bp, synthetic construct
  misc_feature 1..20: note = SNCA1 fwd
  source 1..20: mol_type = other DNA
gactttcaaaggccaaggag

SEQ ID NO: 66 - DNA, 20 bp, synthetic construct
  misc_feature 1..20: note = SNCA1 rev
  source 1..20: mol_type = other DNA
ggagcctacatagagaacac

SEQ ID NO: 67 - DNA, 22 bp, synthetic construct
  misc_feature 1..22: note = SNCA2 fwd
  source 1..22: mol_type = other DNA
attgcagcagccactggctttg

SEQ ID NO: 68 - DNA, 21 bp, synthetic construct
  misc_feature 1..21: note = SNCA2 rev
  source 1..21: mol_type = other DNA
ggcttcaggttcgtagtcttg

SEQ ID NO: 69 - DNA, 24 bp, synthetic construct
  misc_feature 1..24: note = PMP3 fwd
  source 1..24: mol_type = other DNA
cactcatctctatgacgacgtttc

SEQ ID NO: 70 - DNA, 20 bp, synthetic construct
  misc_feature 1..20: note = PMP3 rev
  source 1..20: mol_type = other DNA
caccgtcgagaagctgtaga

SEQ ID NO: 71 - DNA, 267 bp, synthetic construct
  misc_feature 1..267: note = miSNCA_02_miR451
  source 1..267: mol_type = other DNA
gatatcctctggagcctgacaaggaggacaggagagatgctgcaagcccaagaagctctctgctcagcctgtcacaacctactgactgccagggcacttgggaatggcaaggtccttggcctttgaaagtccttactttcaaaggccaaggctcttgctatacccagaaaacgtgccaggaagagaactcaggaccctgaagcagactactggaagggagactccagctcaaacaaggcaggggtgggggcgtgggatggatctcga

SEQ ID NO: 72 - DNA, 267 bp, synthetic construct
  misc_feature 1..267: note = miSNCA5_miR451
  source 1..267: mol_type = other DNA
gatatcctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccctgaagcagactactggaagggagactccagctcaaacaaggcaggggtgggggcgtgggatggatctcga

SEQ ID NO: 73 - DNA, 267 bp, synthetic construct
  misc_feature 1..267: note = miSNCA_07_miR451
  source 1..267: mol_type = other DNA
gatatcctctggagcctgacaaggaggacaggagagatgctgcaagcccaagaagctctctgctcagcctgtcacaacctactgactgccagggcacttgggaatggcaaggtccaacatttgtcacttgctctcaagtgacaaatgttggctcttgctatacccagaaaacgtgccaggaagagaactcaggaccctgaagcagactactggaagggagactccagctcaaacaaggcaggggtgggggcgtgggatggatctcga

SEQ ID NO: 74 - DNA, 267 bp, synthetic construct
  misc_feature 1..267: note = miSNCA_12_miR451
  source 1..267: mol_type = other DNA
gatatcctctggagcctgacaaggaggacaggagagatgctgcaagcccaagaagctctctgctcagcctgtcacaacctactgactgccagggcacttgggaatggcaaggttggttttctcagcagcagccatgctgctgagaaaaccactcttgctatacccagaaaacgtgccaggaagagaactcaggaccctgaagcagactactggaagggagactccagctcaaacaaggcaggggtgggggcgtgggatggatctcga

SEQ ID NO: 75 - DNA, 267 bp, synthetic construct
  misc_feature 1..267: note = miSNCA_13_miR451
  source 1..267: mol_type = other DNA
gatatcctctggagcctgacaaggaggacaggagagatgctgcaagcccaagaagctctctgctcagcctgtcacaacctactgactgccagggcacttgggaatggcaaggaacaccctcttttgtctttcctaagacaaaagagggtgtctcttgctatacccagaaaacgtgccaggaagagaactcaggaccctgaagcagactactggaagggagactccagctcaaacaaggcaggggtgggggcgtgggatggatctcga

SEQ ID NO: 76 - DNA, 267 bp, synthetic construct
  misc_feature 1..267: note = miSNCA_15_miR451
  source 1..267: mol_type = other DNA
gatatcctctggagcctgacaaggaggacaggagagatgctgcaagcccaagaagctctctgctcagcctgtcacaacctactgactgccagggcacttgggaatggcaaggttgcccaactggtcctttttgaaaaggaccagttgggcactcttgctatacccagaaaacgtgccaggaagagaactcaggaccctgaagcagactactggaagggagactccagctcaaacaaggcaggggtgggggcgtgggatggatctcga

SEQ ID NO: 77 - DNA, 267 bp, synthetic construct
  misc_feature 1..267: note = miSNCA_16_miR451
  source 1..267: mol_type = other DNA
gatatcctctggagcctgacaaggaggacaggagagatgctgcaagcccaagaagctctctgctcagcctgtcacaacctactgactgccagggcacttgggaatggcaaggttaggcttcaggttcgtagtcttacgaacctgaagcctactcttgctatacccagaaaacgtgccaggaagagaactcaggaccctgaagcagactactggaagggagactccagctcaaacaaggcaggggtgggggcgtgggatggatctcga

SEQ ID NO: 78 - DNA, 415 bp, synthetic construct
  misc_feature 1..415: note = miR144_miSNCA2+miSNCA5
  source 1..415: mol_type = other DNA
gctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtccttggcctttgaaagtccttactttcaaaggccaaggctcttgctatacccagaaaacgtgccaggaagctgactgccagggcacttgggaatggcaaggtgctctttggtcttctcagccatgagaagaccaaagagcgtcttgctatacccagaaaacgtgccaggaagagaactcaggaccc

SEQ ID NO: 79 - DNA, 828 bp, synthetic construct
  misc_feature 1..828: note = miR144_miSNCA5+miSNCA15
  source 1..828: mol_type = other DNA
gctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagctgactgccagggcacttgggaatggcaaggttgcccaactggtcctttttgaaaaggaccagttgggcactcttgctatacccagaaaacgtgccaggaagagaactcaggacgctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagctgactgccagggcacttgggaatggcaaggttgcccaactggtcctttttgaaaaggaccagttgggcactcttgctatacccagaaaacgtgccaggaagagaactcaggaccc

SEQ ID NO: 80 - DNA, 415 bp, synthetic construct
  misc_feature 1..415: note = miR144_miSNCA5+miSNCA5
  source 1..415: mol_type = other DNA
gctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagctgactgccagggcacttgggaatggcaaggtgctctttggtcttctcagccatgagaagaccaaagagcgtcttgctatacccagaaaacgtgccaggaagagaactcaggaccc

SEQ ID NO: 81 - DNA, 517 bp, synthetic construct
  misc_feature 1..517: note = miR144_miSNCA5+miSNCA15+miSNCA2
  source 1..517: mol_type = other DNA
gctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagctgactgccagggcacttgggaatggcaaggttgcccaactggtcctttttgaaaaggaccagttgggcactcttgctatacccagaaaacgtgccaggaagctgactgccagggcacttgggaatggcaaggtccttggcctttgaaagtccttactttcaaaggccaaggctcttgctatacccagaaaacgtgccaggaagagaactcaggaccc

SEQ ID NO: 82 - DNA, 264 bp, synthetic construct
  misc_feature 1..264: note = miSNCA_01_miR451
  source 1..264: mol_type = other DNA
gatatcctctggagcctgacaaggaggacaggagagatgctgcaagcccaagaagctctctgctcagcctgtcacaacctactgactgccagggcacttgggaatggcaaggcctttgaaagtcctttcatgaatgaaaggactttcaaagctcttgctatacccagaaaacgtgccaggaagagaactcaggaccctgaagcagactactggaagggagactccagctcaaacaaggcaggggtgggggcgtgggatggatcc

SEQ ID NO: 83 - DNA, 264 bp, synthetic construct
  misc_feature 1..264: note = miSNCA_03_miR451
  source 1..264: mol_type = other DNA
gatatcctctggagcctgacaaggaggacaggagagatgctgcaagcccaagaagctctctgctcagcctgtcacaacctactgactgccagggcacttgggaatggcaaggttctcagcagcagccacaactctgtggctgctgctgagactcttgctatacccagaaaacgtgccaggaagagaactcaggaccctgaagcagactactggaagggagactccagctcaaacaaggcaggggtgggggcgtgggatggatcc

SEQ ID NO: 84 - DNA, 264 bp, synthetic construct
  misc_feature 1..264: note = miSNCA_04_miR451
  source 1..264: mol_type = other DNA
gatatcctctggagcctgacaaggaggacaggagagatgctgcaagcccaagaagctctctgctcagcctgtcacaacctactgactgccagggcacttgggaatggcaaggctacatagagaacaccctctttagggtgttctctatgtactcttgctatacccagaaaacgtgccaggaagagaactcaggaccctgaagcagactactggaagggagactccagctcaaacaaggcaggggtgggggcgtgggatggatcc

SEQ ID NO: 85 - DNA, 264 bp, synthetic construct
  misc_feature 1..264: note = miSNCA_06_miR451
  source 1..264: mol_type = other DNA
gatatcctctggagcctgacaaggaggacaggagagatgctgcaagcccaagaagctctctgctcagcctgtcacaacctactgactgccagggcacttgggaatggcaagggtcacttgctctttggtcttctgaccaaagagcaagtgaatcttgctatacccagaaaacgtgccaggaagagaactcaggaccctgaagcagactactggaagggagactccagctcaaacaaggcaggggtgggggcgtgggatggatcc

SEQ ID NO: 86 - DNA, 264 bp, synthetic construct
  misc_feature 1..264: note = miSNCA_09_miR451
  source 1..264: mol_type = other DNA
gatatcctctggagcctgacaaggaggacaggagagatgctgcaagcccaagaagctctctgctcagcctgtcacaacctactgactgccagggcacttgggaatggcaaggcttctgggctactgctgtcacaacagcagtagcccagaactcttgctatacccagaaaacgtgccaggaagagaactcaggaccctgaagcagactactggaagggagactccagctcaaacaaggcaggggtgggggcgtgggatggatcc

SEQ ID NO: 87 - DNA, 264 bp, synthetic construct
  misc_feature 1..264: note = miSNCA_010_miR451
  source 1..264: mol_type = other DNA
gatatcctctggagcctgacaaggaggacaggagagatgctgcaagcccaagaagctctctgctcagcctgtcacaacctactgactgccagggcacttgggaatggcaaggactgtcttctgggctactgctgagtagcccagaagacagctcttgctatacccagaaaacgtgccaggaagagaactcaggaccctgaagcagactactggaagggagactccagctcaaacaaggcaggggtgggggcgtgggatggatcc

SEQ ID NO: 88 - DNA, 264 bp, synthetic construct
  misc_feature 1..264: note = miSNCA_11_miR451
  source 1..264: mol_type = other DNA
gatatcctctggagcctgacaaggaggacaggagagatgctgcaagcccaagaagctctctgctcagcctgtcacaacctactgactgccagggcacttgggaatggcaaggcacattggaactgagcacttgtgtgctcagttccaatgtcacttgctatacccagaaaacgtgccaggaagagaactcaggaccctgaagcagactactggaagggagactccagctcaaacaaggcaggggtgggggcgtgggatggatcc

SEQ ID NO: 89 - DNA, 264 bp, synthetic construct
  misc_feature 1..264: note = miSNCA_14_miR451
  source 1..264: mol_type = other DNA
gatatcctctggagcctgacaaggaggacaggagagatgctgcaagcccaagaagctctctgctcagcctgtcacaacctactgactgccagggcacttgggaatggcaaggggttcgtagtcttgatacccttgtatcaagactacgaacatcttgctatacccagaaaacgtgccaggaagagaactcaggaccctgaagcagactactggaagggagactccagctcaaacaaggcaggggtgggggcgtgggatggatcc

SEQ ID NO: 90 - DNA, 264 bp, synthetic construct
  misc_feature 1..264: note = miSNCA_18_miR451
  source 1..264: mol_type = other DNA
gatatcctctggagcctgacaaggaggacaggagagatgctgcaagcccaagaagctctctgctcagcctgtcacaacctactgactgccagggcacttgggaatggcaaggctccttggttttggagcctacaggctccaaaaccaaggactcttgctatacccagaaaacgtgccaggaagagaactcaggaccctgaagcagactactggaagggagactccagctcaaacaaggcaggggtgggggcgtgggatggatcc

SEQ ID NO: 91 - DNA, 264 bp, synthetic construct
  misc_feature 1..264: note = miSNCA_19_miR451
  source 1..264: mol_type = other DNA
gatatcctctggagcctgacaaggaggacaggagagatgctgcaagcccaagaagctctctgctcagcctgtcacaacctactgactgccagggcacttgggaatggcaaggctttgagaaatgtcatgactggtcatgacatttctcaaactcttgctatacccagaaaacgtgccaggaagagaactcaggaccctgaagcagactactggaagggagactccagctcaaacaaggcaggggtgggggcgtgggatggatcc

SEQ ID NO: 92 - DNA, 612 bp, synthetic construct
  misc_feature 1..612: note = Concatanated SNCA reporter gene
  source 1..612: mol_type = other DNA
ggcgatcgctcgacatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtatgcggccgctggccgcaataaaatatctttattttcattacatctgtgtgttggttttttgtgtg

SEQ ID NO: 93 - DNA, 250 bp, synthetic construct
  misc_feature 1..250: note = miR144_miSCR(ANG1)
  source 1..250: mol_type = other DNA
tggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaagggtagttctattagcgcttactaaagcgctaatagaactaatcttgctatacccaga

SEQ ID NO: 94 - DNA, 415 bp, synthetic construct
  misc_feature 1..415: note = miR144_miSCR(ANG1)+miSCR(C9O)
  source 1..415: mol_type = other DNA
gctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaagggtagttctattagcgcttactaaagcgctaatagaactaatcttgctatacccagaaaacgtgccaggaagctgactgccagggcacttgggaatggcaagggcgacttgtaacgtcagaggcgtctgacgttacaagtcgatcttgctatacccagaaaacgtgccaggaagagaactcaggaccc

SEQ ID NO: 95 - DNA, 313 bp, synthetic construct
  misc_feature 1..313: note = miR144_miSNCA2
  source 1..313: mol_type = other DNA
gctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtccttggcctttgaaagtccttactttcaaaggccaaggctcttgctatacccagaaaacgtgccaggaagagaactcaggaccc

SEQ ID NO: 96 - DNA, 313 bp, synthetic construct
  misc_feature 1..313: note = miR144_miSNCA5
  source 1..313: mol_type = other DNA
gctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtgctctttggtcttctcagccatgagaagaccaaagagcgtcttgctatacccagaaaacgtgccaggaagagaactcaggaccc

SEQ ID NO: 97 - DNA, 313 bp, synthetic construct
  misc_feature 1..313: note = miR144_miSNCA12
  source 1..313: mol_type = other DNA
gctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggttggttttctcagcagcagccatgctgctgagaaaaccactcttgctatacccagaaaacgtgccaggaagagaactcaggaccc

SEQ ID NO: 98 - DNA, 313 bp, synthetic construct
  misc_feature 1..313: note = miR144_miSNCA15
  source 1..313: mol_type = other DNA
gctagcatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggttgcccaactggtcctttttgaaaaggaccagttgggcactcttgctatacccagaaaacgtgccaggaagagaactcaggaccc

SEQ ID NO: 99 - DNA, 687 bp, synthetic construct
  misc_feature 1..687: note = miR144-(miR451_SCR_ANG1)-(miR451_SCR-C9O_1)-(miR451 _SCR_C9O_28)
  source 1..687: mol_type = other DNA
atcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaagggtagttctattagcgcttactaaagcgctaatagaactaatcttgctatacccagaaaacgtgccaggaagctgactgccagggcacttgggaatggcaagggcgacttgtaacgtcagaggcgtctgacgttacaagtcgatcttgctatacccagaaaacgtgccaggaagctgactgccagggcacttgggaatggcaaggattggctcgtctctcatctcgcgatgagagacgagccaactcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgct

SEQ ID NO: 100 - DNA, 267 bp, synthetic construct
  misc_feature 1..267: note = miSCR
  source 1..267: mol_type = other DNA
gatatcctctggagcctgacaaggaggacaggagagatgctgcaagcccaagaagctctctgctcagcctgtcacaacctactgactgccagggcacttgggaatggcaaggtctacatcgtctctatctcttcagatagagacgatgtagctcttgctatacccagaaaacgtgccaggaagagaactcaggaccctgaagcagactactggaagggagactccagctcaaacaaggcaggggtgggggcgtgggatggatctcga

SEQ ID NO: 101 - DNA, 85 bp, synthetic construct
  misc_feature 1..85: note = 144 scaffold with guide strand
  source 1..85: mol_type = other DNA
tggggccctggctgnnnnnnnnnnnnnnnnnnnnnntttgcgatgagacacnnnnnnnnnnnnnnnnnnnnagtccgggcacccc

SEQ ID NO: 102 - DNA, 30 bp, synthetic construct
  misc_feature 1..30: note = Artificial Sequence
  source 1..30: mol_type = other DNA
tgctctttggtcttctcagccatgagaaga

SEQ ID NO: 103 - DNA, 25 bp, synthetic construct
  misc_feature 1..25: note = Artificial Sequence
  source 1..25: mol_type = other DNA
tgctctttggtcttctcagccatga

SEQ ID NO: 104 - DNA, 24 bp, synthetic construct
  misc_feature 1..24: note = Artificial Sequence
  source 1..24: mol_type = other DNA
tgctctttggtcttctcagccatg

SEQ ID NO: 105 - DNA, 23 bp, synthetic construct
  misc_feature 1..23: note = Artificial Sequence
  source 1..23: mol_type = other DNA
tgctctttggtcttctcagccat

SEQ ID NO: 106 - DNA, 17 bp, synthetic construct
  misc_feature 1..17: note = Artificial Sequence
  source 1..17: mol_type = other DNA
gagaagaccaaagagcg

SEQ ID NO: 107 - DNA, 30 bp, synthetic construct
  misc_feature 1..30: note = Artificial Sequence
  source 1..30: mol_type = other DNA
ttgcccaactggtcctttttgaaaaggacc

SEQ ID NO: 108 - DNA, 25 bp, synthetic construct
  misc_feature 1..25: note = Artificial Sequence
  source 1..25: mol_type = other DNA
ttgcccaactggtcctttttgaaaa

SEQ ID NO: 109 - DNA, 24 bp, synthetic construct
  misc_feature 1..24: note = Artificial Sequence
  source 1..24: mol_type = other DNA
ttgcccaactggtcctttttgaaa

SEQ ID NO: 110 - DNA, 23 bp, synthetic construct
  misc_feature 1..23: note = Artificial Sequence
  source 1..23: mol_type = other DNA
ttgcccaactggtcctttttgaa

SEQ ID NO: 111 - DNA, 22 bp, synthetic construct
  misc_feature 1..22: note = Artificial Sequence
  source 1..22: mol_type = other DNA
ttgcccaactggtcctttttga

SEQ ID NO: 112 - DNA, 29 bp, synthetic construct
  misc_feature 1..29: note = Artificial Sequence
  source 1..29: mol_type = other DNA
tccttggcctttgaaagtccttactttca

SEQ ID NO: 113 - DNA, 29 bp, synthetic construct
  misc_feature 1..29: note = Artificial Sequence
  source 1..29: mol_type = other DNA
tccttggcctttgaaagtccttactttct

SEQ ID NO: 114 - DNA, 28 bp, synthetic construct
  misc_feature 1..28: note = TCCTTGGCCTTTGAAAGTCCTTACTTTC
  source 1..28: mol_type = other DNA
tccttggcctttgaaagtccttactttc

(19) United States
(12) Patent Application Publication
(10) Pub. No.: US 2025/0297252 A1
(43) Pub. Date: Sep. 25, 2025

(54) Nucleic acid regulation of SNCA

(71) Applicant: uniQure biopharma B.V., Amsterdam (NL)

(72) Inventors: Astrid Valles Sánchez, Amsterdam (NL); Seyda Acar Broekmans, Amsterdam (NL); Irena Bockaj, Amsterdam (NL); Melvin Maurice Evers, Amsterdam (NL); Morgane Sarah Wartel, Amsterdam (NL); Sebastian Niklas Kieper, Amsterdam (NL)

(73) Assignee: uniQure biopharma B.V., Amsterdam (NL)

(21) Appl. No.: 18/853,724

(22) PCT Filed: Apr. 11, 2023
(86) PCT No.: PCT/EP2023/059361

§ 371 Date: Nov. 5, 2024

(30) Foreign Application Priority Data
Apr. 12, 2022 (EP) 22168001.0

(51) Int. Cl. C12N 15/113

(52) U.S. Cl. CPC C12N 15/113; C12N 2310/11; C12N 2310/531

(57) ABSTRACT

The present invention relates to a nucleic acid comprising two or more RNA encoding sequences, wherein at least one of the sequences comprises a guide sequence substantially complementary to part of the SNCA gene. The invention also relates to associated AAVs, compositions, pharmaceutical compositions and uses in treatments thereof.

### FIELD OF THE INVENTION

The present invention relates to the fields of biotechnology, medicine and gene therapy. Specifically, the invention relates to a nucleic acid comprising two or more RNA encoding sequences, wherein the sequences comprise a guide sequence substantially complementary to part of the α-synuclein (SNCA) gene. The invention also relates to associated AAVs, compositions, pharmaceutical compositions and uses in treatments thereof.

### BACKGROUND OF THE INVENTION

Fibrillar α-synuclein inclusion bodies define two major classes of neurodegenerative disease: Lewy body diseases, including Parkinson's disease (PD) and dementia with Lewy bodies (DLB) and those characterized by Papp-Lantos bodies, including multiple system atrophy (MSA). These are collectively termed synucleinopathies.

PD is a complex progressive neurodegenerative disorder, which includes several motor symptoms such as tremor, rigidity, bradykinesia and/or postural instability. Non-motor symptoms are also observed in PD patients before and/or after the clinical diagnosis. The non-motor symptoms include depression, sleep disturbances, pain and/or fatigue at earlier stages of the disease, anxiety, dementia and/or cognitive dysfunction at later disease stages. Both motor and non-motor symptoms are very debilitating for the patient and their caretakers.

Current therapies focus on short-term relief, focusing on motor symptoms. However, before showing signs of any motor symptoms, PD patients can suffer from non-motor symptoms before diagnosis, and thereby their life quality is significantly diminished, as described above. Hence, symptomatic treatments are not a proactive approach for treating and/or preventing PD.

PD is a complex disorder, and its causes remain unclear. Nonetheless, a number of genes have been found to be involved in the cause and/or progression of PD. One of these genes is the SNCA gene, encoding for the α-synuclein protein.

The main hallmark of PD pathology is the neurodegeneration of dopaminergic neurons in the substantia nigra, which is a mesencephalic brain region with relevant dopaminergic projections to the striatum and cortex, central for motor-related functions. In addition to the loss of nigrostriatal dopaminergic innervation and degeneration in other brain regions, PD is characterized by the presence of cytoplasmic protein aggregates (Lewy bodies) which contain insoluble proteins encoded by the SNCA gene.

As PD progresses, Lewy bodies spread from the first areas including the olfactory bulb and motor nuclei in the brainstem to locus coeruleus and substantia nigra at a later stage, and finally to cortical regions. It was found that the symptoms in different PD stages are associated with specific brain regions where Lewy Bodies spread and/or accumulate. This also means that α-synuclein protein aggregates are correlated with PD, and can result in loss of normal function and/or toxic effects in neurons, which consequently cause neurodegeneration and/or neuroinflammation in different brain regions.

Native α-synuclein protein in the brain is mostly unfolded without a defined tertiary structure. Upon interaction with negatively charged lipids, such as the phospholipids that make up cell membranes, α-synuclein folds into α-helical structures through its N-terminal end. In PD, however, α-synuclein protein adopts a β-sheet-rich amyloid-like structure that is prone to aggregate. The aggregates constitute a major part in Lewy bodies.

MSA is a progressive, adult-onset neurodegenerative disorder of undetermined aetiology characterized by a distinctive oligodendrogliopathy with argyrophilic glial cytoplasmic inclusions (GCIs) and selective neurodegeneration. GCIs or Papp-Lantos inclusions/bodies are now accepted as the hallmarks for the definite neuropathological diagnosis of MSA and suggested to play a central role in the pathogenesis of this disorder. GCIs are composed of hyperphosphorylated α-syn, ubiquitin, LRRK2 (leucin-rich repeat serine/threonine-protein) and other proteins.

Presently, gene therapies for treating and/or preventing a disease are based on completely knocking down a gene and/or transcripts of a gene. The α-synuclein protein encoded by SNCA is considered to be involved in regulation of dopamine release and transport, involved in synaptic transfusion. Therefore, because of the important physiological role of the alpha synuclein protein the depletion of the SNCA transcripts may have a significant impact on the patient's health. Hence, there remains a need for having an optimized and highly potent therapy which can treat and/or prevent different stages of PD and other α-synucleopathies before and/or after clinical diagnosis while minimizing and/or preventing risks.

### SUMMARY OF THE INVENTION

The present invention solves the problem by using nucleic acids which comprising at least two RNA encoding sequences, wherein both sequences comprise a guide sequence substantially complementary to part of an SNCA gene.

The invention provides for a highly versatile system, which allows for the simultaneous use of several guide sequences. The expression of different therapeutic miRNAs from the same vector results in an increased therapeutic efficacy and provides for an optimized and highly potent inhibition of mRNA expression. Consequently, when using the nucleic acids or AAVs of the invention in gene therapy, a single-dose treatment is to be expected. This in turn results in lower toxicity and immunogenicity risks associated with the use of said nucleic acids of the invention. Additionally, the guide sequences may target sequences in different SNCA isoforms that differ qualitatively and quantitatively in their aggregation properties, thereby allowing to reduce expression of RNA encoded by said different isoforms at substantially the same time. Furthermore, the costs of the therapy based on the nucleic acids or AAVs of the invention are expected to be lower than other gene therapy products, presenting a substantial economic advantage.

Thus, a first aspect of the invention relates to a nucleic acid comprising a sequence encoding a first RNA and a sequence encoding a second RNA, wherein each of the first RNA and the second RNA comprise a hairpin, the first RNA comprises a first guide sequence of at least 19 nucleotides substantially complementary to part of an alpha-synuclein (SNCA) gene, and the second RNA comprises a second guide sequence of at least 19 nucleotides substantially complementary to part of an SNCA gene.

A second aspect of the invention relates to an expression cassette comprising the nucleic acid of the invention, where the expression cassette is a DNA molecule.

A third aspect of the invention relates to an adeno-associated virus (AAV) vector (“AAV vector of the invention”) comprising the nucleic acid or expression cassette of the invention.

A fourth aspect of the invention relates to pharmaceutical compositions comprising the nucleic acid, expression cassette, or the AAV vector of the invention.

A fifth aspect of the invention relates to the use as a medicament of the nucleic acid, the expression cassette, the AAV vector or the pharmaceutical composition of the invention.

A sixth aspect of the invention relate to a kit comprising the nucleic acid, the expression cassette, the AAV vector, or pharmaceutical composition of the invention.

A seventh aspect of the invention relates to cells comprising the nucleic acid, the expression cassette or the AAV vector of the invention.

### DESCRIPTION OF THE INVENTION

### Definitions

Unless defined otherwise, technical and scientific terms used herein have the same meaning as commonly understood by one of ordinary skill in the art to which this disclosure belongs. One skilled in the art will recognize many methods and materials similar or equivalent to those described herein, which could be used in the practice of the present invention.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

For the purposes of the present invention, the term “obtained” is considered to be a preferred embodiment of the term “obtainable”. If hereinafter e.g. an antibody is defined to be obtainable from a specific source, this is also to be understood to disclose an antibody which is obtained from this source.

As used herein, the term “and/or” indicates that one or more of the stated cases may occur, alone or in combination with at least one of the stated cases, up to with all of the stated cases.

As used herein, with “At least” a particular value means that particular value or more. For example, “at least 2” is understood to be the same as “2 or more” i.e., 2, 3, 4, 5, 6, 7, 8, 9, 10, 11, 12, 13, 14, 15, . . . etc.

The word “about” or “approximately” when used in association with a numerical value (e.g. about 10) preferably means that the value may be the given value (of 10) more or less 0.1% of the value.

As used herein, “an effective amount” is meant the amount of an agent required to ameliorate the symptoms of a disease relative to an untreated patient. The effective amount of active agent(s) used to practice the present invention for therapeutic treatment of, for example a cancer, varies depending upon the manner of administration, the age, body weight, and general health of the subject. Ultimately, the attending physician or veterinarian will decide the appropriate amount and dosage regimen. Such amount is referred to as an “effective” amount, which (in the case of a viral delivery vector) be determined as genome copies per kilogram (GC/kg). Thus, in connection with the administration of a drug, substance or medicament, which, in the context of the current disclosure, is “effective against” a disease or condition indicates that administration in a clinically appropriate manner results in a beneficial effect for at least a statistically significant fraction of patients, such as an improvement of symptoms, a cure, a reduction in at least one disease sign or symptom, extension of life, improvement in quality of life, or other effect generally recognized as positive by medical doctors familiar with treating the particular type of disease or condition.

The use of a substance as a medicament as described in this document can also be interpreted as the use of said substance in the manufacture of a medicament. Similarly, whenever a substance is used for treatment or as a medicament, it can also be used for the manufacture of a medicament for treatment. Products for use as a medicament described herein can be used in methods of treatments, wherein such methods of treatment comprise the administration of the product for use.

The terms “homology”, “sequence identity” and the like are used interchangeably herein. Sequence identity is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods.

“Sequence identity” and “sequence similarity” can be determined by alignment of two peptide or two nucleotide sequences using global or local alignment algorithms, depending on the length of the two sequences. Sequences of similar lengths are preferably aligned using global alignment algorithms (e.g. Needleman Wunsch) which align the sequences optimally over the entire length, while sequences of substantially different lengths are preferably aligned using local alignment algorithms (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). When sequences have a substantially different overall length, local alignments, such as those using the Smith Waterman algorithm, are preferred.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. 1990 J. Mol. Biol. 215:403-10. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., 1997 Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

Within the context of the present invention, the term “variant thereof”, when referring to a given sequence (SEQ ID), includes any nucleic acids which retain at least some of the properties of the corresponding native nucleic acid, for example, reduction of RNA expression. The term “variant” may include any nucleic acids with at least 50; 55; 60; 65; 70; 75; 80; 90; or 95% sequence identity with the native nucleic acid.

In some embodiments, the sequence of the nucleic acid of the invention is codon optimised. Codon optimisation refers to experimental approaches designed to improve the codon composition of a recombinant gene based on various criteria without altering the amino acid sequence. This is possible because most amino acids are encoded by more than one codon. Most codon-optimization approaches avoid the use of rare codons. However, different approaches vary in the extent of other features considered, including mRNA elements that can inhibit expression, nucleotide context of the initiation codon, mRNA secondary structures, sequence repeats, nucleotide composition, internal ribosome entry sites, promoter sequences, and putative splice donor and acceptor sites. In addition, some programs consider protein structural information, intragenic poly(A) sites, stop codons in alternative reading frames, and dinucleotides that are targets for RNase cleavage, mutation, and methylation-dependent gene silencing. The person skilled in the art has within their understanding the requirements needed to design such a codon-optimised nucleic acid.

As used herein, the term “selectively hybridizing”, “hybridizes selectively” and similar terms are intended to describe conditions for hybridization and washing under which nucleotide sequences at least 66%, at least 70%, at least 75%, at least 80%, more preferably at least 85%, even more preferably at least 90%, preferably at least 95%, more preferably at least 98% or more preferably at least 99% homologous to each other typically remain hybridized to each other. That is to say, such hybridizing sequences may share at least 45%, at least 50%, at least 55%, at least 60%, at least 65, at least 70%, at least 75%, at least 80%, more preferably at least 85%, even more preferably at least 90%, more preferably at least 95%, more preferably at least 98% or more preferably at least 99% sequence identity.

A preferred, non-limiting example of such hybridization conditions is hybridization in 6× sodium chloride/sodium citrate (SSC) at about 45° C., followed by one or more washes in 1×SSC, 0.1% SDS at about 50° C., preferably at about 55° C., preferably at about 60° C. and even more preferably at about 65° C.

Highly stringent conditions include, for example, hybridization at about 68° C. in 5×SSC/5×Denhardt's solution/1.0% SDS and washing in 0.2×SSC/0.1% SDS at room temperature. Alternatively, washing may be performed at 42° C.

The skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), Sambrook and Russell (2001) “Molecular Cloning: A Laboratory Manual (3^{rd} edition), Cold Spring Harbor Laboratory, Cold Spring Harbor Laboratory Press, New York 1995, Current Protocols in Molecular Biology, (John Wiley & Sons, N.Y.).

Of course, a polynucleotide which hybridizes only to a poly A sequence (such as the 3′ terminal poly(A) tract of mRNAs), or to a complementary stretch of T (or U) resides, would not be included in a polynucleotide of the invention used to specifically hybridize to a portion of a nucleic acid of the invention, since such a polynucleotide would hybridize to any nucleic acid molecule containing a poly (A) stretch or the complement thereof (e.g., practically any double-stranded cDNA clone).

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. A “vector” is a nucleic acid construct (typically DNA or RNA) that serves to transfer an exogenous nucleic acid sequence (i.e. DNA or RNA) into a host cell. A vector is preferably maintained in the host by at least one of autonomous replication and integration into the host cell's genome. The terms “expression vector” or “expression construct” refer to nucleotide sequences that are capable of affecting expression of a gene in host cells or host organisms compatible with such sequences. These expression vectors typically include at least one “expression cassette” that is the functional unit capable of affecting expression of a sequence encoding a product to be expressed and wherein the coding sequence is operably linked to the appropriate expression control sequences, which at least comprises a suitable transcription regulatory sequence and optionally, 3′ transcription termination signals. Additional factors necessary or helpful in affecting expression may also be present, such as expression enhancer elements. The expression vector will be introduced into a suitable host cell and be able to affect expression of the coding sequence in an in vitro cell culture of the host cell. A preferred expression vector will be suitable for expression of viral proteins and/or nucleic acids, particularly recombinant AAV proteins and/or nucleic acids.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer or biological entity.

The term “reporter” may be used interchangeably with marker, although it is mainly used to refer to visible markers, such as green fluorescent protein (GFP) or luciferase.

The terms “protein” or “polypeptide” are used interchangeably and refer to molecules consisting of a chain of amino acids, without reference to a specific mode of action, size, 3-dimensional structure or origin.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3-nontranslated sequence (3′-end) comprising a polyadenylation site. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” genetically modified organisms (GMO's) (self-cloning is defined herein as in European Directive 98/81/EC Annex II). When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later. The terms “heterologous” and “exogenous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature.

Heterologous and exogenous nucleic acids or proteins are not endogenous to the cell into which they are introduced but have been obtained from another cell or are synthetically or recombinantly produced. Generally, though not necessarily, such nucleic acids encode proteins, i.e. exogenous proteins, that are not normally produced by the cell in which the DNA is transcribed or expressed. Similarly, exogenous RNA encodes for proteins not normally expressed in the cell in which the exogenous RNA is present. Heterologous/exogenous nucleic acids and proteins may also be referred to as foreign nucleic acids or proteins. Any nucleic acid or protein that one of skill in the art would recognize as foreign to the cell in which it is expressed is herein encompassed by the term heterologous or exogenous nucleic acid or protein. The terms heterologous and exogenous also apply to non-natural combinations of nucleic acid or amino acid sequences, i.e. combinations where at least two of the combined sequences are foreign with respect to each other.

As used herein, the term “non-naturally occurring” when used in reference to an organism means that the organism has at least one genetic alternation that is not normally found in a naturally occurring strain of the referenced species, including wild-type strains of the referenced species. Genetic alterations include, for example, modifications introducing expressible nucleic acids encoding proteins or enzymes, other nucleic acid additions, nucleic acid deletions, nucleic acid substitutions, or other functional disruption of the organism's genetic material. Such modifications include, for example, coding regions and functional fragments thereof for heterologous or homologous polypeptides for the referenced species. Additional modifications include, for example, non-coding regulatory regions in which the modifications alter expression of a gene or operon. Genetic modifications to nucleic acid molecules encoding enzymes, or functional fragments thereof, can confer a biochemical reaction capability or a metabolic pathway capability to the non-naturally occurring organism that is altered from its naturally occurring state.

As used herein, the term “operably linked” refers to a linkage of polynucleotide (or polypeptide) elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

An expression control sequence is “operably linked” to a nucleotide sequence when the expression control sequence controls and regulates the transcription and/or the translation of the nucleotide sequence. Thus, an expression control sequence can include promoters, enhancers, internal ribosome entry sites (IRES), transcription terminators, a start codon in front of a protein-encoding gene, splicing signal for introns, and stop codons.

The term “expression control sequence” is intended to include, at a minimum, a sequence whose presence is designed to influence expression, and can also include additional advantageous components. For example, leader sequences and fusion partner sequences are expression control sequences. The term can also include the design of the nucleic acid sequence such that undesirable, potential initiation codons in and out of frame, are removed from the sequence. It can also include the design of the nucleic acid sequence such that undesirable potential splice sites are removed. It includes sequences or polyadenylation sequences (pA) which direct the addition of a polyA signal, i.e., a string of adenine residues at the 3′-end of a mRNA, sequences referred to as polyA sequences. It also can be designed to enhance mRNA stability. Expression control sequences which affect the transcription and translation stability, e.g., promoters, as well as sequences which affect the translation, e.g., Kozak sequences, are known in insect cells. Expression control sequences can be of such nature as to modulate the nucleotide sequence to which it is operably linked such that lower expression levels or higher expression levels are achieved.

### DETAILED DESCRIPTION OF THE INVENTION

A first aspect of the invention relates to a nucleic acid comprising a sequence encoding a first RNA and a sequence encoding a second RNA, wherein each of the first RNA and the second RNA comprise a hairpin, the first RNA comprises a first guide sequence of at least 19 nucleotides substantially complementary to part of an alpha-synuclein (SNCA) gene, and the second RNA comprises a second guide sequence of at least 19 nucleotides substantially complementary to part of an SNCA gene.

### Nucleic Acids

The term “nucleic acid” as used herein takes its regular meaning in the art. Thus, the term “RNA” or “RNA molecule” or “ribonucleic acid molecule” as used herein refers to a polymer of ribonucleotides (e.g., 2, 3, 4, 5, 10, 15, 20, 25, 30, or more ribonucleotides) and the term “DNA” or “DNA molecule” or “deoxyribonucleic acid molecule” as used herein refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

As used herein, the term “small interfering RNA” (“siRNA”) (also referred to in the art as “short interfering RNAs”) refers to an RNA (or RNA analog) comprising between about 10-50 nucleotides (or nucleotide analogs) which is capable of directing or mediating RNA interference. Preferably, a siRNA comprises between about 15-30 nucleotides or nucleotide analogs, more preferably between about 16-25 nucleotides (or nucleotide analogs), even more preferably between about 18-23 nucleotides (or nucleotide analogs), and even more preferably between about 19-22 nucleotides (or nucleotide analogs) (e.g., 19, 20, 21 or 22 nucleotides or nucleotide analogs). The term “short” siRNA refers to a siRNA comprising about 21 nucleotides (or nucleotide analogs), for example, 19, 20, 21 or 22 nucleotides. The term “long” siRNA refers to a siRNA comprising about 24-25 nucleotides, for example, 23, 24, 25 or 26 nucleotides. Short siRNAs may, in some instances, include fewer than 19 nucleotides, e.g., 16, 17 or 18 nucleotides, provided that the shorter siRNA retains the ability to mediate RNAi. Likewise, long siRNAs may, in some instances, include more than 26 nucleotides, provided that the longer siRNA retains the ability to mediate RNAi absent further processing, e.g., enzymatic processing, to a short siRNA.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be induced, for example, to silence the expression of target genes. Double stranded RNA structures that are suitable for inducing RNAi are well known in the art. For example, a small interfering RNA (siRNA) can induce RNAi. An siRNA comprises two separate RNA strands, one strand comprising a first RNA sequence and the other strand comprising a second RNA sequence, thus a first and a second strand. An siRNA design that is often used involves consecutive base pairs with a 3′ overhang. The first and/or second strand may comprise a 3′-overhang. The 3′-overhang preferably is a dinucleotide overhang on both strands of the siRNA. Such a design is based on observed Dicer processing of larger double stranded RNAs that results in siRNAs having these features. The 3-overhang may be comprised in the first strand. The 3′-overhang may be in addition to the first strand. The length of the two strands of which an siRNA is composed may be 19, 20, 21, 22, 23, 24, 25, 26 or 27 nucleotides or more.

siRNAs may also serve as Dicer substrates. For example, a Dicer substrate may be a 27-mer consisting of two strands of RNA that have 27 consecutive base pairs. The first strand is positioned at the 3′-end of the 27-mer duplex. At the 3′-ends, like with siRNAs, each or one of the strands may comprise a two nucleotide overhang. The 3-overhang may be comprised in the first strand. The 3′-overhang may be in addition to the first strand. 5′ from the first strand, additional sequences may be included that are either complementary to the target RNA sequence adjacent or not. The other end of the siRNA Dicer substrate is blunt ended. This Dicer substrate design may result in a preference in processing by Dicer such that an siRNA can be formed like the siRNA design as described above, having 19 consecutive base pairs and 2 nucleotide overhangs at both 3′-ends.

In any case, siRNAs, or the like, are composed of two separate RNA strands (Fire et al. 1998, Nature 19; 391 (6669):806-11), each RNA strand comprising or consisting of the first and second RNA strand or the third and fourth RNA strand in accordance with to the invention. Thus, the nucleic acid of the invention may be said to derive a first and second RNA strand, which is the first RNA, and a third and fourth RNA strand, which is the second RNA. Alternative naming conventions for each first, second, third and fourth RNA strands are within the scope of the invention, which may be complementary, substantially complimentary, or unique to each other, or in any other required arrangement as discussed herein.

The loop sequence may also be a stem-loop sequence, whereby the double stranded region of the shRNA is extended. Like the siRNA Dicer substrate described above, a shRNA can be processed by e.g. Dicer to provide for an siRNA having an siRNA design such as described above, having e.g. 19 consecutive base pairs and 2 nucleotide overhangs at both 3′-ends. In case the shRNA is to be processed by Dicer, it is preferred to have the first and second strands at the end of the shRNA, i.e. such that the putative strands of the siRNA are linked via a stem loop sequence: 5′-first strand-stem loop sequence-second strand-optional 2 nt overhang sequence-3′. Or, conversely, 5′-second strand-stem loop sequence-first strand-optional 2 nt overhang sequence-3′. Another shRNA design may be a shRNA structure that is processed by the RNAi machinery to provide for an activated RNA-induced silencing complex (RISC) that does not require Dicer processing (Liu et al., 2013 Nucleic Acids Res. 41(6):3723-33, incorporated herein by reference), so called AgoshRNAs or Ago2 processed RNAs, which are based on a structure very similar to the miR-451 scaffold as described below. Such a shRNA structure comprises in its loop sequence part of the first RNA sequence. Such a shRNA structure may also consist of the first strand, followed immediately by the second strand.

### Spacers

In preferred embodiments of the invention, the sequence encoding the first RNA is followed by a spacer comprising at least 15 nucleotides and the sequence encoding the second RNA. Thus, preferably, in a 5′ to 3′ direction, the sequence encoding the first RNA is followed by a first spacer comprising at least 15 nucleotides, which spacer is followed by the sequence encoding the second RNA. It is understood that the 5′ to 3′ direction refers to the coding strand in case of a double-stranded (ds) nucleic acid.

As described above, the nucleic acid may be said to derive a first and second RNA strand, which is the first RNA, and a third and fourth RNA strand, which is the second RNA, wherein the sequences encoding the first and second RNA strands are followed by a spacer of at least 15 nucleotides and the sequences encoding the third and fourth RNA strands.

In some embodiments, the nucleic acid of the invention comprises a third sequence encoding a third RNA.

In preferred embodiments of the invention, the sequence encoding the third RNA is followed by a second spacer comprising at least 15 nucleotides followed by the sequence encoding the first RNA. Thus, preferably, in a 5′ to 3′ direction, the sequence encoding the third RNA is followed by a second spacer comprising at least 15 nucleotides, which second spacer is followed by and the sequence encoding the first RNA, followed by the first spacer and the sequence encoding the second RNA.

Thus, the nucleic acid may be said to derive a first and second RNA strand, which is the first RNA, a third and fourth RNA strand, which is the second RNA, and a fifth and sixth RNA strands, which is the third RNA, wherein preferably, in a 5′ to 3′ direction, the sequences encoding the fifth and sixth RNA strands are followed by a spacer of at least 15 nucleotides followed by the sequences encoding the first and second RNA strands, said first and second strands followed by a spacer of at least 15 nucleotides and the sequences encoding the third and fourth RNA strands.

In some embodiments of the invention, the spacer comprises at least 25; at least 30; or at least 35 nucleotides.

In some embodiments of the invention, the spacer comprises at least: 15; 20; 25; 30; 35; 40; 45; 50; 55; 60; 65; 70; 75; 80; 85; 90; 95; 100; 105; 110; 115; 120; 125; 130; 135; 140; 145; 150; 155; 160; 165; 170; 175; 180; 185; 190; 195; or 200 nucleotides.

In some specific embodiments, the spacer of the invention comprises 15; 16; 17; 18; 19; 20; 21; 22; 23; 24; 25; 26; 27; 28; 29; 30; 31; 32; 33; 34; 35; 36; 37; 38; or 40 nucleotides.

In some specific embodiments of the invention, the spacer comprises 75; 76; 77; 78; 79; 80; 81; 82; 83; 84; 85; 86; 87; 88; 89; 90; 91; 92; 93; 94; 95; 96; 97; 98; 99; 100; 101; 102; 103; 104; or 105 nucleotides.

In some embodiments of the invention, the spacer comprises or consists of a sequence selected from the group consisting of: SEQ ID NO. 1; SEQ ID NO. 2; SEQ ID NO. 3; and variants thereof.

Thus, in some specific embodiments of the invention, the sequences encoding the first and second RNA strands are followed by a spacer of at least 15 nucleotides and the sequences encoding the third and fourth RNA strands.

In some specific embodiments of the invention, the sequences encoding the first and second RNA strands are followed by a spacer comprising or consisting of SEQ ID NO. 3 and the sequences encoding the third and fourth RNA strands.

Thus, in some specific embodiments of the invention, the sequences encoding the first and second RNA strands are followed by a spacer of at least 75 nucleotides and the sequences encoding the third and fourth RNA strands.

In some specific examples of the invention, the sequences encoding the first and second RNA strands are followed by a spacer comprising or consisting of SEQ ID NO. 1 and the sequences encoding the third and fourth RNA strands.

In some specific embodiments, the sequences encoding the fifth and sixth RNA strands are followed by a spacer of at least 75 nucleotides and the sequences encoding the first and second RNA strands are followed by a spacer of at least 15 nucleotides and the sequences encoding the third and fourth RNA strands. In some specific examples of the invention, the sequences encoding the fifth and sixth RNA strands are followed by a spacer comprising or consisting of SEQ ID NO. 1 and the sequences encoding the first and second RNA strands are followed by a spacer comprising or consisting of SEQ ID NO. 3 and the sequences encoding the third and fourth RNA strands.

In some embodiments, if one or both RNAs are shRNA to be processed by Dicer, the relevant shRNA structures mentioned above are also applicable. In some embodiment, if one or both RNAs are Ago shRNAs or Ago2 processed RNAs, the relevant shRNA structures mentioned above are also applicable. Thus, one or both RNAs may be processed by the same or different RNAi machinery.

### RNA Scaffolds

A double stranded RNA according to the invention may be incorporated in a pre-miRNA or pri-miRNA scaffold. MicroRNAs, i.e. miRNA, are guide strands that originate from double stranded RNA molecules that are endogenously expressed e.g. in mammalian cells. A miRNA is processed from a pre-miRNA precursor molecule, similar to the processing of a shRNA or an extended siRNA as described above, by the RNAi machinery and incorporated in an activated RISC (Tijsterman M, Plasterk R H. Dicers at RISC; the mechanism of RNAi. Cell. 2004 Apr. 2; 1 17(1):1-3). A pre-miRNA is a hairpin RNA molecule that can be part of a larger RNA molecule (pri-miRNA), e.g. comprised in an intron, which is first processed by Drosha to form a pre-miRNA hairpin molecule. The pre-miRNA molecule is a shRNA-like molecule that can subsequently be processed by Dicer to result in an siRNA-like double stranded RNA duplex. The miRNA, i.e. the guide strand, that is part of the double stranded RNA duplex is subsequently incorporated in RISC.

The first and second RNA of the invention comprise a first and a second guide sequence of at least 19 nucleotides substantially complementary to part of an SNCA gene.

An RNA molecule such as present in nature, i.e. a pri-miRNA, a pre-miRNA or a miRNA duplex, may be used as a scaffold for producing an artificial miRNA that specifically targets a gene of choice. Based on the predicted RNA structure of the RNA molecule as present in nature, e.g. as predicted using e.g. m-fold software using standard settings (Zuker. Nucleic Acids Res. 31 (13), 3406-3415, 2003), the natural miRNA sequence as it is present in the RNA structure (i.e. duplex, pre-miRNA or primiRNA), and the sequence present in the structure that is substantially complementary therewith are removed and replaced with a first strand and a second strand according to the invention, that are the first strand and second strand of the first RNA, or the first strand and second strand of the second RNA, which may also be referred to as the third and fourth strands. Thus, using the first and second strand for solely exemplary purpose, the first strand and the second strand are preferably selected such that the predicted secondary RNA structures that are formed, i.e. of the pre-miRNA, pri-miRNA and/or miRNA duplex, resemble the corresponding predicted original secondary structure of the natural RNA sequences. pre-miRNA, pri-miRNA and miRNA duplexes (that consist of two separate RNA strands that are hybridized via complementary base pairing) as found in nature, are often not fully base paired, i.e. not all nucleotides that correspond with the first and second strand as defined above are base paired, and the first and second strand are often not of the same length. How to use miRNA precursor molecules as scaffolds for any selected target RNA sequence and substantially complementary first strand is described e.g. in Liu Y P Nucleic Acids Res. 2008 36(9):281 1-24.

A pri-miRNA can be processed by the RNAi machinery of the cell. The pri-miRNA comprising flanking sequences at the 5′-end and the 3′-end of a pre-miRNA hairpin and/or shRNA like molecule. Such a pri-miRNA hairpin can be processed by Drosha to produce a pre-miRNA. The length of the flanking sequences can vary but may be around 80 nt in length (Zeng and Cullen, J Biol Chem. 2005 280(30):27595-603; Cullen, Mol Cell. 2004 16(6):861-5). The minimal length of the single-stranded flanks can easily be determined as when it becomes too short, the RNA molecule may lose its function because e.g. Drosha processing fails resulting in sequence specific inhibition being reduced or even absent. In one embodiment, the pri-miRNA scaffold carrying the first and second strand according to the invention has a 5′-sequence flank and a 3′ sequence flank relative to the predicted pre-miRNA structure of at least 5, at least 10, at least 15, at least 20, at least 30, at least 40, or at least 50 nucleotides. Preferably, the pri-miRNA derived flanking sequences (5′ and 3′) comprised in the miRNA scaffold are derived from the same naturally occurring pri-miRNA sequence. Preferably, pre-miRNA and/or the pri-miRNA derived flanking sequences (5′ and 3′) and/or loop sequences comprised in the miRNA scaffold are derived from the same naturally occurring pri-miRNA sequence. As the (putative) guide strand RNA as comprised in the endogenous miRNA sequence can be replaced by a sequence including (or consisting of) the first strand, and the passenger strand sequence replaced by a sequence including (or consisting of) the second strand, it is understood that flanking sequences and/or loop sequences of the pri-miRNA or pre-miRNA sequences of the endogenous sequence may include minor sequence modifications such that the predicted structure of the scaffold miRNA sequence (e.g. M-fold predicted structure) is the same as the predicted structure of the endogenous miRNA sequence.

The first and second strands and the third and fourth strands, such as to form two double stranded RNAs, i.e. the first and second RNA of the invention, are encoded by an expression cassette. It is understood that, unless otherwise stated, any additional RNAs comprised in the nucleic acid of the invention, such as the third RNA of the invention as described below, are also encoded by the expression cassette. It is also understood that when the double stranded RNAs are to be e.g. two siRNAs, consisting of two strands each, that there may be two or more expression cassettes required. When each double stranded RNA is comprised in a single RNA molecule, e.g. encoding a shRNA, pre-miRNA or pri-miRNA, one expression cassette per RNA molecule may suffice. A pol II expression cassette may comprise a promoter sequence a sequence encoding an RNA to be expressed followed by a polyadenylation sequence. In case the double stranded RNAs that are expressed comprise a pri-miRNA scaffold, the encoded RNA sequence may encode for intron sequences and exon sequences and 3′-UTR's and 5′-UTRs. A pol III expression cassette in general comprises a promoter sequence, followed by a sequence encoding an RNA (e.g. shRNA sequence, pre-miRNA, or a strand of the double stranded RNAs to be comprised in e.g. an siRNA or 5 extended siRNA). A pol I expression cassette may comprise a pol I promoter, followed by an RNA encoding sequence and a 3′-sequence. Expression cassettes for double stranded RNAs are well known in the art, and any type of expression cassette can suffice, e.g. one may use a pol III promoter, a pol II promoter or a pol I promoter (i.a. ter Brake et al., 2008 Mol Ther. March; 16(3):557-64, Maczuga et al., 2012 BMC Biotechnol. July 24; 12:42). In some embodiments, and as further described below, the expression cassette is a DNA molecule. In some specific embodiments, the expression cassette comprises a pol II promoter.

As is clear from the above, the first and second strands comprised in a double stranded RNA can contain additional nucleotides and/or nucleotide sequences. The double stranded RNA of the invention may be comprised in a single RNA sequence or comprised in two separate RNA strands. Whatever design is used, it is designed such that, from the RNA sequence, an antisense RNA molecule comprising the first or the second strand, as further explained below, in whole or a substantial part thereof, can be processed by the RNAi machinery, such that it is incorporated in the RISC complex to have its action, i.e. to induce RNAi against the RNA target sequence comprised in an RNA encoded by the SNCA gene. The sequence comprising or consisting of the first or second strand, in whole or a substantial part thereof, is capable of sequence specifically targeting RNA encoded by a human SNCA gene. Hence, as long as the double stranded RNA is capable of inducing RNAi, such a double stranded RNA is contemplated in the invention.

In some embodiments, the double stranded RNA according to the invention is comprised in a pre-miRNA scaffold, a pri-miRNA scaffold, a shRNA, or an siRNA. Preferably, the first and second strand, or the third and fourth strands, or the fifth and sixth strands, or all strands as encoded by the expressed cassette are to be contained in a single transcript. It is understood that the expressed transcript in subsequent processing, i.e. cleavage, results in the single transcript being processed into multiple separate RNA molecules.

The term complementary is herein defined herein as nucleotides of a nucleic acid sequence that can bind to another nucleic acid sequence through hydrogen bonds, i.e. nucleotides that are capable of base pairing. Ribonucleotides, the building blocks of RNA are composed of monomers (nucleotides) containing a sugar, phosphate and a base that is either a purine (guanine, adenine) or pyrimidine (uracil, cytosine). Complementary RNA strands form double stranded RNA. A double stranded RNA may be formed from two separate complementary RNA strands or the two complementary RNA strands may be comprised in one RNA strand. In complementary RNA strands, the nucleotides cytosine and guanine (C and G) can form a base pair, guanine and uracil (G and U), and uracil and adenine (U and A) can form a base pair as well. The term substantial complementarity means that is not require do have the first and second RNA sequence to be fully complementary, or to have the first RNA sequence and target RNA sequence or sequences of RNA encoded by a human SNCA gene to be fully complementary.

### Guide Strands

The first and second RNA to be expressed in accordance with the invention comprise, in whole or a substantial part thereof, a guide strand.

In certain embodiments of the invention, the guide strand may also be referred to as antisense strand as it is complementary (“anti”) to a target RNA sequence in a sense transcript, the sense target RNA sequence being comprised in an RNA encoded by a SNCA gene. Thus, the first and second RNA also comprise a “sense strand”, that may have substantial sequence identity with, or be identical to, the target RNA sequence. Therefore, the first and second RNA can be described as a hairpin or a double stranded RNA that is substantially complementary to itself.

Said double stranded RNA according to the invention is to induce RNA interference, thereby reducing expression of SNCA transcripts, which includes knocking down of SNCA derived transcripts. Transcripts that may be targeted may include spliced, including splice variants, and unspliced RNA transcripts. Thus, an RNA encoded by a human SNCA gene is understood to comprise unspliced mRNAs comprising a 5′ untranslated region (UTR), intron and exon sequences, followed by a 3′ UTR and a polyA signal, and also splice variants thereof. The double stranded RNA according to the invention may also induce transcriptional silencing.

Reducing expression of an SNCA transcript is herein thus preferably understood as reducing the steady state level of a functional SNCA mRNA in a target cell such that less of the mRNA is available in the cell for translation into the α-synuclein protein, thereby reducing the steady state level of the protein in the target cell. Reducing expression of an SNCA transcript therefore does not necessarily involve reducing de novo transcription of the SNCA gene but rather increased degradation of an SNCA mRNA and/or its precursors, e.g. unspliced RNA transcripts.

In some embodiments, the double stranded RNA according to the invention comprises a first RNA sequence and a second RNA sequence, i.e. the first and second or third and fourth RNA strands, wherein the first and second RNA sequences are substantially complementary, and wherein the first RNA sequence has a sequence length of at least 19 nucleotides and is substantially complementary to a target RNA sequence of an RNA encoded by a SNCA gene, which first RNA sequence is capable of inducing RNA interference to sequence-specifically reduce expression of an RNA transcript comprising the target RNA sequence. In some further embodiments, said induction of RNA interference to reduce expression of an RNA transcript comprising the target RNA sequence means that it is to reduce SNCA gene expression.

In some embodiments, the double stranded RNA according to the invention comprises a first RNA sequence and a second RNA sequence, i.e. the first and second or third and fourth RNA strands, wherein the first and second RNA sequences are substantially complementary, and wherein the second RNA sequence has a sequence length of at least 19 nucleotides and is substantially complementary to a target RNA sequence of an RNA encoded by a SNCA gene, which second RNA sequence is capable of inducing RNA interference to sequence-specifically reduce expression of an RNA transcript comprising the target RNA sequence. In some further embodiments, said induction of RNA interference to reduce expression of an RNA transcript comprising the target RNA sequence means that it is to reduce SNCA gene expression.

The skilled person can easily determine whether this is the case by using standard luciferase reporter assays and appropriate controls such as described in the examples and as known in the art (Zhuang et al. 2006 Methods Mol Biol. 342:181-7). For example, a luciferase reporter comprising a target RNA sequence can be used to show that the double stranded RNA according to the invention is capable of sequence specific knock down. Furthermore, levels of SNCA expression can be determined by detecting endogenous SNCA mRNA, α-synuclein protein (soluble, aggregated or phosphorylated forms), and/or α-synuclein protein isoforms (SNCA140, SNCA126, SNCA112, SNCA98; SEQ ID NO. 35, 36, 37 and 38, respectively). Levels of SNCA mRNA or α-synuclein protein can be determined in different sample types such as cell lysates, tissue lysates, blood cells and biofluids such as serum, plasma and cerebrospinal fluid.

As used herein, the term “RNA silencing” refers to a group of sequence-specific regulatory mechanisms (e.g. RNA interference (RNAi), transcriptional gene silencing (TGS), post-transcriptional gene silencing (PTGS), quelling, co-suppression, and translational repression) mediated by RNA molecules which result in the inhibition or “silencing” of the expression of a corresponding protein-coding gene. RNA silencing has been observed in many types of organisms, including plants, animals, and fungi.

It is understood that “substantially complementary” in this context means that it is not required to have all the nucleotides of the guide sequence and the target sequence to be base paired, i.e. to be fully complementary, or all the nucleotides of the guide sequence and the target sequence to be base paired. As long as the first, second, and where applicable, third RNAs of the invention are capable of inducing RNA interference to thereby sequence-specifically target a sequence comprising the target RNA sequence, such substantial complementarity is contemplated in accordance with the invention.

The substantial complementarity between the strand complimentary to the target RNA sequence, also referred to as part of an SNCA gene, preferably consists of at most two mismatched nucleotides, more preferably having one mismatched nucleotide, most preferably having no mismatches. It is understood that one mismatched nucleotide means that over the entire length of the strand complimentary to the target RNA sequence when base paired with the target RNA sequence one nucleotide does not base pair with the target RNA sequence. Having no mismatches means that all nucleotides of the strand complimentary to the target RNA base pair with the target RNA sequence, having 2 mismatches means two nucleotides of the strand complimentary to the target RNA do not base pair with the target RNA sequence. The strand complimentary to the target RNA may also comprise additional nucleotides that do not have complementarity to the target RNA sequence, and may be longer than e.g. 21 nucleotides. In such a scenario, the substantial complementarity is determined over the entire length of the target RNA sequence. This means that the target RNA sequence in this embodiment has either no, one or two mismatches over its entire length when base paired with the strand complimentary to the target RNA.

Substantial complementarity between the strand complimentary to the target RNA and the target RNA sequence consists of having none, one or two mismatches over the entire length of either the strand complimentary to the target RNA or the target RNA sequence encoded by an RNA of the human SNCA, whichever is the shortest.

As said, a mismatch according to the invention means that a nucleotide of the first (or second) and third strand (the strand) does not base pair with the target RNA sequence encoded by an RNA of the first or second gene. Nucleotides that do not base pair are A and A, G and G, C and C, U and U, A and C, C and U, or A and G. A mismatch may also result from a deletion of a nucleotide, or an insertion of a nucleotide. When the mismatch is a deletion in the strand sequence, this means that a nucleotide of the target RNA sequence is not base paired with the sequence when compared with the entire length of the strand sequence. Nucleotides that can base pair are A-U, G-C and G-U. A G-U base pair is also referred to as a G-U wobble, or wobble base pair. In one embodiment the number of G-U base pairs between the strand sequence and the target RNA sequence is 0, 1 or 2.

In some embodiments, there are no mismatches between the strand RNA sequence and the target RNA sequence and a G-U base pair or G-U pairs are allowed. Preferably, there may be no G-U base pairs between the strand sequence and the target RNA sequence, or the strand sequence and the target RNA sequence only have base pairs that are A-U or G-C. Preferably, there are no G-U base pairs and no mismatches between the strand sequence and the target RNA sequence. The strand sequence of the double stranded RNA according to invention preferably is fully complementary to the target RNA sequence, said complementarity consisting of G-U, G-C and A-U base pairs. The strand sequence of the double stranded RNA according to invention more preferably is fully complementary to the target RNA sequence, said complementarity consisting of G-C and A-U base pairs.

Therefore, in some embodiments, the strand sequence and the target RNA sequence have at least 15, 16, 17, 18, or 19 nucleotides that base pair. Preferably the strand and the target RNA sequence are substantially complementary, said complementarity comprising at least 19 base pairs. In other embodiments, the strand has at least 8, 9, 10, 11, 12, 13 or 14 consecutive nucleotides that base pair with consecutive nucleotides of the target RNA sequence. In other embodiments, the strand has at least 19 consecutive nucleotides that base pair with consecutive nucleotides of the target RNA sequence. In other embodiments, the strand comprises at least 19 consecutive nucleotides that base pair with 19 consecutive nucleotides of the target RNA sequence. In other embodiments, the strand has at least 17 nucleotides that base pair with the target RNA sequence and have at least 15 consecutive nucleotides that base pair with consecutive nucleotides of the target RNA sequence. The sequence length of the first strand is preferably at most 21, 22, 23, 24, 25, 26, or 27 nucleotides. In other embodiments, the strand has at least 20 consecutive nucleotides that base pair with 20 consecutive nucleotides of the target RNA sequence. In other embodiments, the strand comprises at least 21 consecutive nucleotides that base pair with 21 consecutive nucleotides of the target RNA sequence.

As said, it may be not required to have full complementarity (i.e. full base pairing (no mismatches) and no G-U base pairs) between the first or second strand of the first RNA and the first strand of the second RNA, and the target RNA sequence as such a strand can still allow for sufficient suppression of gene expression. Also, not having full complementarity may be contemplated for example to avoid or reduce off-target RNA sequence specific gene suppression while maintaining sequence specific inhibition of transcripts comprising the target RNA sequence. However, it may be preferred to have full complementarity as it may result in more potent inhibition. Without being bound by theory, having full complementarity between the first or second strand of the first RNA and the first strand of the second RNA, and the target RNA sequence may allow for the activated RISC complex comprising said first or second strand of the first RNA or the first strand of the second RNA (or a substantial part thereof) to cleave its target RNA sequence, whereas having mismatches may hamper cleavage and can result in mainly allowing inhibition of translation, of which the latter may result in less potent inhibition.

With regard to the second strand on the first or second RNA, the second strand is substantially complementary with the first strand. The second strand combined with the first strand forms a double stranded RNA. As said, this is to form a suitable substrate for the RNA interference machinery such that a guide sequence derived from the first strand is comprised in the RISC complex in order to sequence specifically inhibit expression of its target, i.e. RNA encoded by a human SNCA gene. The sequence of the second strand has sequence similarity with the target RNA sequence. However, the substantial complementarity of the second strand with the first strand may be selected to have less substantial complementarity as compared with the substantial complementarity between the first strand and the target RNA sequence. Hence, the second strand may comprise 0, 1, 2, 3, 4, or more mismatches, 0, 1, 2, 3, or more GU wobble base pairs, and may comprise insertions of 0, 1, 2, 3, 4, nucleotides and/or deletions of 0, 1, 2, 3, 4, nucleotides. It is understood that, where the guide sequence is comprised within the second strand of the first RNA, or the second strand of the invention, the description above applies to the first strand of the invention.

Preferably the first strand and the second strand are substantially complementary, said complementarity comprising 0, 1, 2 or 3 G U base pairs and/or wherein said complementarity comprises at least 17 base pairs. These mismatches, G-U wobble base pairs, insertions and deletions, are with regard to the first strand, i.e. the double stranded region that is formed between the first and second strands. As long as the first and second strands can substantially base pair, and are capable of inducing sequence specific inhibition of an RNA encoded by a human SNCA gene, such substantial complementarity is allowed according to the invention. It is also understood that substantially complementarity between the first and the second strands may depend on the double stranded RNA design of choice. It may depend for example on the miRNA scaffold that is chosen for in which the double stranded RNA is to be incorporated.

As clear from the above, the substantial complementarity between the first strand and second strand of the first and second RNA may comprise mismatches, deletions and/or insertions relative to a first and second RNA sequence being fully complementary (i.e. fully base paired). In some embodiments, the first and second strands of the first and/or second RNA have at least 11 consecutive base pairs. Hence, at least 11 consecutive nucleotides of the first strand and at least 11 consecutive nucleotides of the second strand are fully complementary. In some embodiment the first and second strands of the first and/or second RNA have at least 15 nucleotides that base pair. Said base pairing between at least 15 nucleotides of the first strand and at least 15 nucleotides of the second strand may consist of G-U, G-C and A-U base pairs, or may consist of G-C and A-U base pairs.

In some embodiments, the first and second RNA sequences have at least 15 nucleotides that base pair and have at least 11 consecutive base pairs. In another embodiments, the first RNA sequence and the second RNA sequence are substantially complementary, wherein said complementarity comprises at least 17 base pairs. Said 17 base pairs may preferably be 17 consecutive base pairs, said base pairing consisting of G-U, G-C and A-U base pairs or consisting of G-C and A-U base pairs.

The invention thus provides for an expression cassette encoding the first strand and second strand of the first and/or second RNA wherein the first and second strands are substantially complementary, wherein the first strand has a sequence length of at least 19 nucleotides and is substantially complementary to a target RNA sequence comprised in an RNA encoded by a human SNCA gene. Suitable target RNA sequences in accordance with the invention are provided (see e.g. table 1). Where applicable, the expression cassette may also encode the first strand and second strand of the third RNA, wherein the first and second strands are substantially complementary.

In certain embodiments of the invention, the first RNA comprises a guide sequence substantially complementary to a first target sequence in its second strand. In said cases, the embodiments above apply to the second and third strands of the invention instead of the first and third strands of the invention.

TABLE 1
Suitable target RNA sequences of
transcripts encoded by the human SNCA gene.

| SEQ | | | length |
|---|---|---|---|
| ID | | Target Sequence | (nucleo- |
| NO. | miSNCA | (5′-sequence-3′) | tides) |
| 4 | 02 | AAGGACTTTCAAAGGCCAAGGA | 22 |
| 5 | 05 | TGGCTGAGAAGACCAAAGAGCA | 22 |
| 6 | 07 | AGAGCAAGTGACAAATGTTGGA | 22 |
| 7 | 12 | TGGCTGCTGCTGAGAAAACCAA | 22 |
| 8 | 13 | AGGAAAGACAAAAGAGGGTGTT | 22 |
| 9 | 15 | TCAAAAAGGACCAGTTGGGCAA | 22 |
| 10 | 16 | AGACTACGAACCTGAAGCCTAA | 22 |

In some embodiments, an expression cassette is provided encoding a first strand and a second strand wherein the first and second strands of the first and/or second RNA are substantially complementary, wherein the first strand has a sequence length of at least 22 nucleotides and is substantially complementary to a target RNA sequence selected from the group listed in table 1 comprised in an RNA encoded by a human SNCA gene.

Similarly, in some embodiments, an expression cassette is provided encoding a first strand and a second strand wherein the first and second strands of the first and/or second RNA are substantially complementary, wherein the second strand of the first RNA and the first strand of the second RNA have a sequence length of at least 22 nucleotides and is substantially complementary to a target RNA sequence selected from the group listed in table 1. Where applicable, the expression cassette may also encode the first strand and second strand of the third RNA, wherein the first and second strands are substantially complementary.

In some embodiments of the invention, each of the guide sequences is substantially complementary to a sequence selected from the group consisting of: SEQ ID NO. 4 to SEQ ID NO. 10.

In some embodiments of the invention, the first and second guide sequences of the invention are the same sequence. In other embodiments, the first and second guide sequences of the invention are different sequences.

As stated above, the RNAs of the invention may be incorporated into miRNA scaffolds. The miRNA scaffold sequence is processed by the RNAi machinery as present in the cell. In some specific embodiments of the invention, the processing of the miRNA scaffold sequence results in: guide sequences comprising the first strand of the first RNA, or a substantial part thereof, in the range of 21-30 nucleotides; and guide sequences comprising the first strand of the second RNA, or a substantial part thereof, in the range of 21-30 nucleotides. In some other specific embodiments of the invention, the processing of the miRNA scaffold sequence results in: guide sequences comprising the second strand of the first RNA, or a substantial part thereof, in the range of 21-30 nucleotides; and guide sequences comprising the first strand of the second RNA, or a substantial part thereof, in the range of 21-30 nucleotides.

Such guide strands are capable of reducing the SNCA gene transcript expression by targeting the selected target sequences.

As shown in the examples, the first strand of the first RNA and the first strand of the second RNA, or, alternatively, the second strand of the first RNA and the first strand of the second RNA, as it is encoded by the expression cassette of the invention, is comprised in part or in whole, in a guide strand when it has been processed by the RNAi machinery of the cell. Hence, the guide strand that is to be generated from the RNA encoded by the expression cassette, comprising the first or second strand of the first RNA and the first strand of the second RNA, is to comprise at least 18 nucleotides of the second RNA sequence.

Preferably, such a guide strand comprises at least 19 nucleotides, 20 nucleotides, 21 nucleotides, or 22 nucleotides. A guide strand can comprise the first or second strand of the first or the first strand of the second RNA sequence, also as a whole. In selecting a miRNA scaffold, the first or second strand of the first RNA sequence and the first strand in the second RNA sequence, can be selected such that it is to replace the original guide strand. However, this does not necessarily mean that the guide strand produced from such an artificial scaffold is identical in length to the first or second strand of the first RNA or the first strand of the second RNA selected, nor that the first or second strand of the first RNA or the first strand of the second RNA is in its entirety to be found in the guide strand that is produced.

miR-451

In some embodiments of the invention, each of the first RNA and the second RNA is incorporated in a pre-miRNA or a pri-miRNA scaffold derived from miR-451. In some specific embodiments of the invention, each of the first RNA and the second RNA comprises SEQ ID NO. 11 or a variant thereof.

Thus, and as shown in the examples, the first, second, third and fourth strands of the nucleic acid of the invention may be incorporated in a pre-miRNA or a pri-miRNA scaffold derived from miR-451. The miR-451 scaffold is found to be in particular useful within the present invention as it can induce RNA interference that can result in mainly guide strand induced RNA interference. The pri-miR-451 scaffold does not result in a passenger strand because the processing is different from the canonical miRNA processing pathway (Cheloufi et al. 2010 Nature 465(7298):584-9 and Yang et al., 2010 Proc Natl Acad Sci USA 107(34):15163-8). The scaffolds represents an excellent candidate to develop a gene therapy product as unwanted potential off-targeting by passenger strands can be largely, if not completely, avoided. As the passenger strand (corresponding to the second sequence) may result in targeting of transcripts other than SNCA RNA, using such scaffolds may prevent such unwanted targeting. Hence, it is preferred that selected scaffolds produce less than 15%; less than 10%; less than 5%; less than 4%; or less than 3% of passenger strands.

A miRNA 451 scaffold, as shown in the examples, preferably comprises from 5′ to 3′, firstly 5′-CUUGGGAAUGGCAAGG-3′ (SEQ ID NO. 12), followed by a sequence of 22 nucleotides, comprising or consisting of the first RNA sequence, followed a sequence of 17 nucleotides, which can be regarded as the second RNA sequence, which is complementary over its entire length with nucleotides 2-18 of said sequence of 22 nucleotides, subsequently followed by sequence 5′-SWCUUGCUAUACCCAGA-3′ (wherein S is an A or a G or a C and W is an A or a U) (SEQ ID NO. 13). Preferably the first 5′-G/C/A nucleotide of the latter sequence is not to base pair with the first nucleotide of the first strand of the first or second RNA.

Such a scaffold may comprise further flanking sequences as found in the original pri-miR-451 scaffold. Alternatively, the hairpin stem sequences, 5′-CUUGGGAAUGGCAAGG-3′ (SEQ ID NO. 12) and 5′-SWCUUGCUAUCCCAGA-3′ (SEQ ID NO. 13) may be replaced by hairpin stem sequences of other pri-mRNA structures. As is clear from the above, the sequence of the scaffold may differ not only with regard to the (putative) guide strand sequence, and sequence complementary thereto, as present in the wild-type scaffold, but may also comprise additional mutations in the 5′, loop and 3′ sequence as well, as additional mutations may be required to provide for an RNA structure that is predicted to mimic the secondary structure of the wild-type scaffold. Such a scaffold may be comprised in a larger RNA transcript, e.g. a pol II expressed transcript, comprising e.g. a 5′ UTR and a 3′UTR and a poly A. Flanking structures may also be absent.

An expression cassette in accordance with the invention thus expressing a shRNA-like structure having a sequence of 22 nucleotides, comprising or consisting of the first strand of the second RNA, followed a sequence of 17 nucleotides, which can be regarded to be the second strand of the second RNA, which is complementary over its entire length with nucleotides 2-18 of said sequence of 22 nucleotides. The latter shRNA-like structure derived from the miR-451 scaffold can be referred to as a pre-miRNA scaffold from miR-451.

In some embodiments, an expression cassette according to the invention is provided, wherein said each of the first strand of the first and second RNA are substantially complementary to a target RNA sequence comprised in antisense RNA transcripts encoded by the human SNCA gene.

In some embodiments, each of the first strand of the first and second RNA is substantially complementary to a target sequence selected from the group consisting of SEQ ID NO. 4 to 10. In some preferred embodiments, each of the first strand of the first and second RNA has a length of 19, 20, 21, or 22 nucleotides. In some specific embodiments, each the first strand of the first and second RNA is fully complementary over its entire length with the target sequence. In some preferred embodiments, each of the first strand of the first and second RNA has a length of 19, 20, 21, or 22 nucleotides, wherein said first strand of the first and second RNA is fully complementary over its entire length with the target sequence. Each of the first strand of the first and second RNA can be selected from the group consisting of SEQ ID NO. 14 to SEQ ID NO. 20.

As described above, the first strand of each of the first and second RNA is to be combined with a second strand of the first and second RNA. As described herein, the skilled person is well capable of designing and selecting a suitable second strand of the first and second RNA in order to provide for a first and second strand for the first and second RNA that can induce RNA interference when expressed in a cell. Suitable second strands of the first and second RNA can be selected from the group consisting of SEQ ID NO. 21 to SEQ ID NO. 27.

The first strand of the first and second RNA is comprised in a miR-451 scaffold, such as shown in the examples. A suitable scaffold comprising a first and second strand for the first and second RNA in accordance with the invention can be a sequence such as SEQ ID NO. 11.

The first strand of the first and/or second RNA as described above can be comprised in expression cassettes. The first strand of the first and/or second RNA can also be comprised in RNA structures that are encoded by expression cassettes.

The first and second strands of the first and second RNA sequence as described above can be comprised in expression cassettes. The first and second strands of the first and second RNA can also be comprised in RNA structures that are encoded by expression cassettes. It is understood that the cassettes may additionally comprise the first and second strands of the third RNA, where present, and as described above.

Targeting the above-disclosed RNA sequences, utilizing such first and second RNA, was found to be particularly useful for reducing expression of RNA transcripts encoded by the human SNCA gene. By targeting human SNCA this way, the current inventors were able to highly efficiently reduce human SNCA gene expression and thus to reduce the expression of SNCA RNA and α-synuclein protein and eventually reduce the formation of α-synuclein aggregates and Lewy and/or Papp-Lantos bodies. Ultimately this may reverse, prevent, slow down the progression of, or completely halt any pathologies related to the SNCA gene.

In some embodiments, the nucleic acid of the invention comprises a third sequence encoding a third RNA. In some specific embodiments, the third RNA comprises SEQ ID NO. 34 or a variant thereof.

Thus, the nucleic acid may be said to derive a first and second RNA strand, which is the first RNA, a third and fourth RNA strand, which is the second RNA, and a fifth and second RNA strands, which is the third RNA, wherein the sequences encoding the fifth and sixth RNA strands are followed by: a spacer of at least 15 nucleotides and the sequences encoding the first and second RNA strands followed by a spacer of at least 15 nucleotides and the sequences encoding the third and fourth RNA strands.

Said third RNA may be incorporated in a pre-miRNA or a pri-miRNA scaffold derived from miR-144. In some specific embodiments of the invention, the third RNA comprises SEQ ID NO. 34 or a variant thereof. In some embodiments, the variant thereof is SEQ ID NO. 28. In other words, and as described above, the first and second strands of the third RNA of the invention are incorporated in a pre-miRNA or a pri-miRNA scaffold derived from miR-144. Thus, the third RNA can be described as a hairpin or a double stranded RNA that is substantially complementary to itself. In some embodiments, the hairpin in the second RNA comprises at least 70 nucleotides.

In said embodiments where the third RNA is incorporated in a pre-miRNA or a pri-miRNA scaffold derived from miR-144, the third RNA is processed by Dicer; therefore, the putative strands of the subsequent siRNA are linked via a stem loop sequence: 5′-first strand-apical loop sequence-second strand-optional 2 nt overhang sequence-3′ or, conversely, 5′-second strand-apical loop sequence-first strand-optional 2 nt overhang sequence-3′.

In some embodiments of the invention where the third RNA is incorporated in a pre-miRNA or a pri-miRNA scaffold derived from miR-144, the third RNA is mutated to reduce processing and/or expression of the third RNA. In some specific embodiments, SEQ ID NO. 34 or the variant thereof is mutated to reduce processing and/or expression of the third RNA.

In some embodiments, the mutation is a single point mutation. In other words, the first RNA comprises a single point mutation to reduce processing and/or expression of the first RNA. Any mismatch, bulge or GU wobble introduced within positions 4-8 of the DROSHA cleavage site may impair the enzymatic activity of DROSHA. Double and triple mismatches, bulges or wobbles within said positions further decrease the activity of DROSHA. Therefore, any of the following single nucleotide polymorphisms (SNPs) and combinations thereof within the 4-8 nucleotide stretch of mir-144 may alter (pre-)-mir-144 expression.

In some embodiments, the third RNA comprises at least one mutation selected from the group consisting of: U>G at position 4; A>U or G at position 5; U>A at position 6; C>G or U at position 7; and A>U or G at position 8.

In preferred embodiments, the third RNA comprises a single point mutation is A>U at position 5. The skilled person can easily determine whether this is the case by using standard assays and appropriate controls such as described in the examples and as known in the art.

The specific combination of miR-144/miR-451 scaffolds within the nucleic acid of the invention is particularly helpful within the context of gene therapy and RNA silencing. miR-144 and miR-451 are examples of clustered miRNAs regulated in trans, wherein miR-144 regulates the processing of miR-451 by Ago2. Specifically, miR-144 enhances miR-451 biogenesis in trans by repressing Dicer and, in turn, repressing global canonical miRNA processing (Kretov et al., 2020 Molecular Cell 78, 317-328). Thus, where this combination of scaffolds is used, the third RNA of the invention plays a key role in enhancing the biogenesis of the first and second RNA of the invention, and therefore, the delivery of the guide sequences comprised in said first and second RNAs.

In some embodiments of the invention, the first RNA is incorporated into a miR-144 scaffold.

In some specific embodiments of the invention, the first RNA is incorporated into a miR-144 scaffold, and the second RNA is incorporated into a miR-451 scaffold.

As stated above, the first RNA comprises a first guide sequence. In those embodiments of the invention where the first RNA is incorporated into a miR-144 scaffold, the first guide sequence may be incorporated into the first or the second strand of the first RNA.

In those embodiments where the first RNA of the invention is incorporated into a miR-144 scaffold, the miRNA-144 scaffold for use in the invention preferably comprises from 5′ to 3′, firstly 5′-UGGGGCCCUGGCUM-3′ (wherein M is an A or a C or a G or a U) (SEQ ID NO. 29), followed by a sequence of 22 nucleotides, comprising or consisting of a first RNA sequence, followed by a sequence of 15 nucleotides which can be regarded as the apical loop 5′-UUUGCGAUGAGAWMM-3′ (wherein W is preferably a C or a G, but can also be a A or a U, and is to base pair with the last nucleotide of the first strand of the first RNA; and wherein M is an A or a C and is not to base pair with nucleotide 21 of the first strand of the first RNA) (SEQ ID NO. 30), followed by a sequence of 20 nucleotides, which can be regarded as the second RNA sequence, and which is complementary over its entire length with nucleotides 1 and 3-10 and 12-20 of said sequence of 22 nucleotides, except nucleotide 18 that forms a mismatch with nucleotide 2 of the first strand, subsequently followed by sequence 5′-AGUCCGGGCACCCCC-3′ (SEQ ID NO.31).

Preferably the first 5′-U nucleotide of the latter sequence is not to base pair with the first nucleotide of the first strand of the third RNA. Such a scaffold may comprise flanking sequences as found in the original pri-miR-144 scaffold. Alternatively, the flanking sequences 5′-ATCGGCGCTATGCTTCCTGTGCCCCCAG-3′ (SEQ ID NO 32) and 5′-AGCTCTGGAGCCTGACAAGGAGGACAGGAGAGATGCTGCAAGCCCAAGAAGCTCTCTGCTC AGCCTGTCACAACCTACTGACTGCCAGGGCA-3′ (SEQ ID NO. 33) may be replaced by flanking sequences of other pri-mRNA structures. Flanking structures may also be absent.

In those embodiments where the first RNA of the invention comprises a miR-144 scaffold, the guide sequence may be comprised within the 5p- or 3p-arm of the scaffold. DROSHA processing of miR-144, subsequently followed by DICER processing, generate a miRNA duplex which ultimately enters the process of miRNA strand selection. Strand selection is operated within the RISC and determines which strand will become the active strand (also referred to as the guide strand) and which strand will be degraded (passenger strand) (Noland and Doudna, 2013 RNA, 19:639-648). Guide strand selection is highly determined by thermodynamic characteristics of the miRNA duplex, and thus it may be influenced by modifying the nucleotide sequence encoding the miRNA. Generally speaking, the strand with the lower thermodynamic stability at its 5′ end will become the guide strand. Another key feature of human miRNA guide strands is a 5′ end U-bias associated with an enrichment for A and G nucleotides, whilst the passenger strands exhibit a 5′ end C-bias and an enrichment for C and U nucleotides (Hai Yang Hu et al. 2009, BMC Genomics 2009, 10:413).

Conveniently, since the guide strand selection is sequence dependent, the miR-144 constructs of the invention comprising guide strands may be designed as 5p- or 3p-guide-containing regions (SEQ ID NO 101). Importantly, the probability with which the desired guide strand of the invention will be selected in RISC can be modulated by single nucleotide variations such as selecting U as the first nucleotide of the first strand (5p-design) or the first nucleotide of the second strand (3p-designs) and selecting C or G as nucleotide 20 of the first strand (5p-designs) or nucleotide 19 of the second strand (3p-designs).

As with miR-451 scaffolds, the sequence of the miR-144 scaffold may differ not only with regard to the (putative) guide strand sequence, and sequence complementary thereto, as present in the wild-type scaffold, but may also comprise additional mutations in the 5′, loop and 3′ sequence as well, as additional mutations may be required to provide for an RNA structure that is predicted to mimic the secondary structure of the wild-type scaffold. Again, such a scaffold may be comprised in a larger RNA transcript, e.g. a pol II expressed transcript, comprising e.g. a 5′ UTR and a 3′UTR and a poly A. Flanking structures may also be absent. An expression cassette in accordance with the invention may thus express a shRNA-like structure having a sequence of 22 nucleotides, comprising or consisting of the first strand of the first RNA, followed a sequence of 17 nucleotides, which can be regarded to be the second strand of the first RNA, which is complementary over its entire length with nucleotides 1 and 3-10 and 11-19 of said sequence of 22 nucleotides, except nucleotide 18 that forms a mismatch with nucleotide 2 of the first strand. The latter shRNA-like structure derived from the miR-144 scaffold can be referred to as a pre-miRNA scaffold from miR-144.

In some embodiments, an expression cassette according to the invention is provided, wherein said first or second strand of the first RNA is substantially complementary to a target RNA sequence comprised in RNA transcripts encoded by the SNCA gene.

In some preferred embodiments, the first or the second strand of the first RNA has a length of 19, 20, 21, or 22 nucleotides. In some specific embodiments, the first or second strand of the first RNA is fully complementary over its entire length with the first target sequence. In some preferred embodiments, the first or the second strand of the first RNA has a length of 19, 20, 21, or 22 nucleotides, wherein said first strand of the first RNA is fully complementary over its entire length with the target sequence.

As explained above, where the first strand of the first RNA comprises a guide sequence, the first strand is to be combined with a second strand of the first RNA. As above, the skilled person is well capable of designing and selecting a suitable second strand of the first RNA.

Similarly, where the second strand of the first RNA comprises a guide sequence, the second strand is to be combined with a first strand of the first RNA. As above, the skilled person is well capable of designing and selecting a suitable first strand of the first RNA

Thus, in some embodiments of the invention, the first RNA comprises SEQ ID NO. 101 or a variant thereof. In some more specific embodiments of the invention, the first RNA comprises SEQ ID NO. 101 or a variant thereof; and the second RNA comprises SEQ ID NO. 11 or a variant thereof.

In some embodiments of the invention, where the first RNA comprises SEQ ID NO. 101, the first guide sequence is incorporated into the first strand of the first RNA; in other embodiments, the guide sequence is incorporated into the second strand of the first RNA.

In some embodiments of the invention, the guide sequences are selected from the group consisting of SEQ ID NO. 14 to SEQ ID NO. 20. In some embodiments, the complementary strand sequences to SEQ ID NO. 14 to 20 are SEQ ID NO. 21 to SEQ ID NO. 27, respectively.

In some embodiments of the invention, the first RNA comprises a sequence selected from the group consisting of SEQ ID NO. 71 to SEQ ID NO. 77. In some embodiments of the invention, the second RNA comprises a sequence selected from the group consisting of SEQ ID NO. 71 to SEQ ID NO. 77.

In some embodiments of the invention, the nucleic acid comprises a sequence selected from the group consisting of SEQ ID NO. 78 to SEQ ID NO. 81 and SEQ ID NO. 95 to SEQ ID NO. 98.

In some embodiments of the invention, the first and second RNA comprise SEQ ID NO. 11; and the sequence encoding the first RNA is followed by a spacer which comprises SEQ ID NO. 3.

In some embodiments of the invention, the first RNA comprises SEQ ID NO. 101 and the second RNA comprises SEQ ID NO. 11; and the sequence encoding the first RNA is followed by a spacer which comprises or consist of SEQ ID NO. 1.

In some embodiments of the invention, the third RNA comprises SEQ ID NO. 34 and the second and third RNA comprise SEQ ID NO. 11; the sequence encoding the third RNA is followed by a spacer which comprises or consist of SEQ ID NO. 1; and the sequence encoding the first RNA is followed by a spacer which comprises or consist of SEQ ID NO. 3.

In a second aspect of the invention, there is provided an expression cassette comprising the nucleic acid of the invention, wherein the expression cassette is a DNA molecule. In some specific embodiments, the nucleic acid comprised within the cassette is operably linked to a promoter and optionally to a poly-A signal. In some embodiments, the expression cassette comprises, in 5′ to 3′ order, the following elements: at least one promoter, at least a first and second RNA and at least a poly A signal.

The nucleotide sequence comprising an expression cassette or expression cassettes as defined herein above for expression in a mammalian cell comprises at least one mammalian cell-compatible expression control sequence, e.g. a promoter, that is/are operably linked to the sequence coding for the gene product of interest, thus forming an expression cassette for expression of the gene product of interest in mammalian target cell to be treated by gene therapy with the gene product of interest. Many such promoters are known in the art (see Sambrook and Russel, 2001, supra). Constitutive promoters that are broadly expressed in many cell-types, such as the CMV promoter, may be used. However, more preferred will be promoters that are inducible, tissue-specific, cell-type-specific, or cell cycle-specific. Preferably a pol II promoter is used, such as a CAG promoter (i.a. Miyazaki et al. 1989 Gene 79 (2): 269-77; Niwa, Gene. 108 (2): 193-9), a PGK promoter, or a CMV promoter (Such as depicted e.g. in FIG. 2 of WO2016102664). As PD and any atypical presentations mimicking other kinds of neurodegenerative brain diseases primarily affect the brain, it may be particularly useful to use a neuron-specific promoter.

Thus, in some embodiments of the invention, the promoter is a promoter capable of driving transcription in a brain cell. Examples of suitable neuron-specific promoters are Neuron-Specific Enolase (NSE), human synapsin 1, CaMKII kinase, native or engineered chicken beta-actin (CAG) promoter, human synapsin I with a minimal CMV sequence (Synl-minCMV), platelet-derived growth factor-beta chain (PDGF), tyrosine hydroxylase (TH), Forkhead Box A2 (FOXA2) and tubulin alpha (Hioki et al. 2007 Gene Ther. 14(11):872-82). Other suitable promoters that can be contemplated are inducible promoters, i.e. a promoter that initiates transcription only when the host cell is exposed to some particular stimulus.

The expression cassette comprising the nucleic acid of the invention encodes a polyA signal comprised operably linked to the 3′ end of the RNA molecule. In some embodiments of the invention, the polyA signal is the simian virus 40 polyadenylation (SV40 polyA); or Bovine Growth Hormone polyadenylation (bGH polyA); or human growth hormone polyadenylation (hGH polyA).

The expression cassettes according to the invention can be transferred to a cell, using e.g. transfection methods. Any suitable means may suffice to transfer an expression cassette according to the invention. Preferably, the expression cassettes according to the invention are comprised in a viral vector, preferably a gene therapy vector. Preferably, gene therapy vectors are used that stably transfer the expression cassette to the cells such that stable expression of the double stranded RNAs that induce sequence specific inhibition of the SNCA can be achieved. Suitable vectors may be lentiviral vectors, retrotransposon based vector systems, or AAV vectors. It is understood that as e.g. lentiviral vectors carry an RNA genome, the RNA genome will encode for the said expression cassette such that after transduction of a cell, the said DNA sequence and said expression cassette is formed. Preferably, the gene therapy vector is a viral vector. Preferably the viral vector is an AAV. Therefore, in some embodiments, an expression cassette as disclosed herein is flanked by Inverted Terminal Repeats.

Thus, in preferred embodiments of the invention, the expression cassette comprising the nucleic acid of the invention is flanked by at least one AAV Inverted Terminal Repeats (ITRs). In some specific embodiments, the expression cassette is flanked by one 5′ ITR and one 3′ ITR. In other words, the expression cassette is flanked by an ITR sequence at the 5′ end of the cassette and an ITR sequence at the 3′ end of the cassette.

### AAVs

A third aspect of the present invention relates to an adeno-associated virus (AAV) comprising the nucleic acid or expression cassette of the invention.

Recombinant parvoviruses, in particular dependoviruses such as infectious human or simian adeno-associated virus (AAV), and the components thereof (e.g. a parvovirus genome), may be used as vectors for introduction and/or expression of nucleic acids in mammalian cells, preferably human cells. An “AAV vector” is defined as a recombinantly produced AAV or AAV particle that comprises a polynucleotide to be delivered into a host cell, either in vivo, ex vivo or in vitro. Herein, an AAV vector construct refers to the polynucleotide comprising the viral genome or part thereof, usually at least one ITR, and a transgene. Herein, a transgene refers to a nucleotide sequence of interest and may comprise promotor and/or regulatory sequences necessary for expression as well as sequences encoding the gene of interest.

A “recombinant parvoviral or AAV vector” (or “rAAV vector”) or a “parvoviral or AAV vector” herein refers to a parvoviral or AAV virion (i.e. a capsid), comprising (or ““packaging”) one or more nucleotide sequences of interest, genes of interest or “transgenes” that is/are flanked by at least one parvoviral or AAV inverted terminal repeat sequence (ITR). Preferably, the transgene(s) is/are flanked by ITRs, one on each side of the transgene(s). Such (r)AAV vectors can be replicated and packaged into infectious viral particles when present in a suitable host cell that is expressing AAV rep and cap gene products (i.e. AAV Rep and Cap proteins). When the transgene(s) of interest that is/are flanked by at least one ITR is incorporated into a larger nucleic acid construct (e.g. in a chromosome or in another vector such as a plasmid or baculovirus used for cloning or transfection), this is typically referred to as a “pro-vector” which can be “rescued” by replication and encapsidation in the presence of AAV packaging functions and necessary helper functions.

Preferably the AAV vector that is used is an AAV vector of serotype 5 or 9. AAV of serotype 5 or 9 (also referred to as AAV5 and AAV9) may be in particularly useful for transducing human neurons and human astrocytes such as shown in the examples. Therefore, in some embodiment, there is an AAV comprising an expression cassette as disclosed herein. Thus, AAV5 and AAV9 can efficiently transduce different human cell types of the CNS including FBN, dopaminergic neurons, motor neurons and astrocytes and is therefore a suitable vector candidate to deliver therapeutic genes to the CNS to treat neurogenerative diseases, including but not limited to the treatment of PD via targeting e.g. SNCA gene as described herein. The production of AAV vectors comprising any expression cassette of interest is well described in, WO2007/046703, WO2007/148971, WO2009/014445, WO2009/104964, WO2011/122950, WO2013/0361 18, which are incorporated herein in its entirety.

AAV sequences that may be used in the present invention for the production of AAV vectors, e.g. produced in insect or mammalian cell lines, can be derived from the genome of any AAV serotype. Generally, the AAV serotypes have genomic sequences of significant homology at the amino acid and the nucleic acid levels, provide an identical set of genetic functions, produce virions which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chlorini et al. (1997, J. Vir. 71: 6823-33); Srivastava et al. (1983, J. Vir. 45:555-64); Chlorini et al. (1999, J. Vir. 73:1309-1319); Rutledge et al. (1998, J. Vir. 72:309-319); and Wu et al. (2000, J. Vir. 74: 8635-47). AAV serotypes 1, 2, 3, 4 and 5 are preferred source of AAV nucleotide sequences for use in the context of the present invention. Preferably the AAV ITR sequences for use in the context of the present invention are derived from AAV1, AAV2, and/or AAV5. Likewise, the Rep52, Rep40, Rep78 and/or Rep68 coding sequences are preferably derived from AAV1, AAV2 and AAV5. The sequences coding for the VP1, VP2, and VP3 capsid proteins for use in the context of the present invention may however be taken from any of the known 42 serotypes, more preferably from AAV1, AAV2, AAV3, AAV4, AAV5, AAV6, AAV7, AAV8 or AAV9 or newly developed AAV-like particles obtained by e.g. capsid shuffling techniques and AAV capsid libraries. AAV capsids may consist of VP1, VP2 and VP3, but may also consist of VP1 and VP3.

In some embodiments, the AAV vector according to the inventions comprises an AAV5 or AAV9 capsid proteins. In some embodiment, the AAV vector according to the inventions comprises a AAV5 capsid protein. In some embodiment, the AAV vector according to the inventions comprises a AAV9 capsid protein.

In another embodiment, a host cell is provided comprising the said nucleic acid or said expression cassette according to the invention. For example, the said expression cassette or nucleic acid may be comprised in a plasmid contained in bacteria. Said expression cassette or nucleic acid may also be comprised in a production cell that produces e.g. a viral vector. Said expression cassette may also be provided in a baculovirus vector.

Various modifications of the nucleotide sequences as defined above, including e.g. the wildtype AAV sequences, for proper expression in the host cell is achieved by application of well-known genetic engineering techniques such as described e.g. in Sambrook and Russell (2001, supra). Various further modifications of coding regions are known to the skilled artisan which could increase yield of the encode proteins. These modifications are within the scope of the present invention.

In one embodiment, any mammalian cell may be infected by an AAV vector of the invention, for example, but not limited to, a muscle cell, a liver cell, a nerve cell, a glial cell and an epithelial cell and the mammalian species may be any species including, but not limited to, murine, dog, non-human primate and human. In a preferred embodiment the cell to be infected is a human cell.

It is to be understood that the capsid amino acid sequences and the nucleotide sequences encoding them can be engineered, for example, the sequence may be a hybrid form or may be codon optimized, such as for example by codon usage of AcmNPv or Spodoptera frugiperda. The capsid proteins may be engineered, for example, via DNA shuffling, error prone PCR, bioinformatics rational design or site saturated mutagenesis. Resulting capsids are based on the existing serotypes but contain various amino acid or nucleotide changes that improve the features of such capsids. The resulting capsids can be a combination of various parts of existing serotypes, “shuffled capsids” or contain completely novel changes, i.e. additions, deletions or substitutions of one or more amino acids or nucleotides, organized in groups or spread over the whole length of gene or protein. See for example Schaffer and Maheshri; Proceedings of the 26th Annual International Conference of the IEEE EMBS San Francisco, CA, USA; Sep. 1-5, 2004, pages 3520-3523; Asuri et al., 2012 Molecular Therapy 20(2):329-3389; Lisowski et al., 2014, Nature 506(7488):382-386, herein incorporated by reference.

In some embodiments, the ITRs and capsid proteins (or parts thereof) in the AAV vector of the invention may be from different AAV serotypes. By way of example, and not limitation, the ITRs may be derived from AAV2, whilst the capsid proteins may be derived from a different serotype, for example AAV5 or AAV9.

In a fourth aspect, the present invention relates to a pharmaceutical composition (“the pharmaceutical composition of the invention”) comprising the nucleic acid of the invention, the expression cassette, or the AAV vector of the invention, and at least one pharmaceutically acceptable excipient.

Suitable excipients include, but are not limited to, buffers and stabilizers, antioxidants, etc. By way of example, the pharmaceutical composition of the invention may comprise physiological buffers, such as e.g. PBS, and stabilizing agents such as e.g. sucrose.

The compositions of the invention are compatible with and suitable and intended for use in subsequent intravenous, intrastriatal, intracerebellar, intrathecal, intraparenchymal, intravitreal, subretinal administration or for use in organ-targeted vascular delivery such as intraportal or intracoronary delivery or isolated limb perfusion.

In some embodiments, the pharmaceutical composition of the invention may also comprise at least one immunosuppressive compound. Said compound may reduce and/or prevent an immune response induced by administration of the pharmaceutical composition of the invention.

In some embodiments these compositions are used to transduce cells in vitro or ex vivo, in which case the excipients will need to be compatible with cell culture.

In a fifth aspect, the invention relates to the nucleic acid of the invention, the expression cassette of the invention, the AAV vector of the invention, or the pharmaceutical composition of the invention for use as a medicament. The appropriate definitions are provided elsewhere in this application.

In some embodiments, said medicament reduces expression of RNA encoded by the human SNCA gene, as explained herein above.

In some embodiments, at least one of the guide sequences is substantially complementary to part of an exon comprised in the human SNCA gene. Said exon may be selected from the group consisting of: exon 2; exon 4; and exon 6.

In some specific embodiments, the medicament reduces expression of transcripts encoded by at least one splice-site variant of the SNCA gene. In some more specific embodiments, the at least one splice-site variant is selected from the group consisting of: SNCA140, SNCA 112, SNCA126, and SNCA98.

The human SNCA gene encodes different isoforms through alternative splicing. SNCA isoforms SNCA140 (SEQ ID NO. 35), SNCA126 (SEQ ID NO. 36), SNCA112 (SEQ ID NO. 37), SNCA98 (SEQ ID NO. 38) are encoded by SNCA nucleic acids comprising common exons such as exons 2, 4 and 6. These four isoforms differ qualitatively and quantitatively in their aggregation properties. Canonical isoform SNCA140 is more prone to aggregation than SNCA126, SNCA112 and/or SNCA98. Moreover, in aggregates, SNCA140 forms relatively straight fibrils while SNCA126 forms shorter fibrils, which are arranged in parallel fibril bundles, and SNCA98 forms annular structures. Pathologic aggregation of SNCA proteins is a central process in the pathogenesis of PD. Thus, using the nucleic acids of the present invention allows to reduce expression of different isoforms at substantially the same time, providing for a highly potent method for treating and/or preventing PD.

Within the context of this invention, “potency” is understood as an expression of the activity of a medicament or therapeutic substance, in terms of the concentration or amount needed to produce a defined effect. The potency of a given medicament may be expressed as the concentration (EC50) or dose (ED50) of the drug required to produce 50% of that medicament's maximal effect.

In certain cases, relative potency may be used, where instead of using units to describe the dose required to achieve a certain endpoint, a ratio of equivalent doses is used. By way of example, medicament A is 10 times more potent than medicament B i.e., it achieves the same effect with 1/10^{th} of the dose.

In some specific embodiments, the medicament decreases or knocks down the amount of α-syn protein aggregates and/or the amount of Lewy and/or Papp-Lantos bodies.

In some embodiments of the invention, the medicament is used for treating and/or preventing PD, DLB, MSA, neuropsychiatric symptoms, motor symptoms of PD, cognitive impairment, sleep disturbances, autonomic disturbances, and/or olfactory disturbance.

As used herein, the term “treat” and any of its variants refers to any kind of healthcare which is intended to relieve or eliminate the symptoms and/or causes of illness, injury, mental health problems, etc. The term “prevent” and any of its variants refers to any action taken to decrease the chance of getting a disease or condition.

It is understood that treatment of PD, LBD, MSA, neuropsychiatric symptoms, motor symptoms of PD, cognitive impairment, sleep disturbances, autonomic disturbances, and/or olfactory disturbance may be provided not only to human subjects suffering from any of said diseases, but also to human subjects having a genetic predisposition of developing the diseases who may or may not show symptoms of the disease. These subjects include, without limitation, subjects with known duplication or triplication of SNCA gene, or with disease-causing mutations in SNCA gene. i.e. A30P, E46K, H50Q, G51D and A53T.

In certain embodiments, the nucleic acid of the invention, the AAV of the invention or the pharmaceutical composition of the invention need to be delivered to a target cell for their use as a medicament. As stated above, PD, DLB, MSA, neuropsychiatric symptoms, motor symptoms of PD, cognitive impairment, sleep disturbances, autonomic disturbances, and/or olfactory disturbance primarily affect the central nervous system (CNS). Thus, in some embodiments of the invention, the target cell is a CNS cell. In some embodiments of the invention, the target cell is a neuron. In some specific embodiments of the invention, the target cell is a brainstem neuron or a midbrain neuron or a hippocampal neuron or an amygdala neuron and/or cerebral cortex neuron.

In certain embodiments, the nucleic acid of the invention, the AAV of the invention or the pharmaceutical composition of the invention are delivered to the cerebrospinal fluid (CSF).

In certain embodiments, the nucleic acid of the invention, the AAV of the invention or the pharmaceutical composition of the invention are delivered to the substantia nigra and/or to the striatum and/or to the thalamus.

In certain embodiments of the invention, the AAV of the invention or the pharmaceutical composition of the invention are delivered to the CNS target cell by injection. In certain embodiments, the injection is a intraparenchymal injection. In some embodiments, the injection is an intrathecal injection. In some other embodiments, the injection is a cisterna magna injection. In some other embodiments, the injection is an intracerebroventricular injection. In some other embodiments, the injection is a subpial injection. In certain embodiments of the invention, the injections are MRI-guided injections. In certain embodiments of the intervention, the injections are followed by focused ultrasound (fUS).

In certain embodiments, the AAV of the invention or the pharmaceutical composition of the invention are delivered to the CNS target cell by a combination of delivery methods. By way of a non-limiting example, the combination of delivery methods may comprise intrathecal or subpial injection combined with intracerebroventricular and/or intrastriatal injection; or intrathecal or subpial injection combined with intraparenchymal injections. In some embodiments of the invention, the AAV of the invention or the pharmaceutical composition of the invention are delivered by convection enhanced delivery

The methods for producing the nucleic acid of the invention comprise any methods for producing nucleic acids, including but not limited to de novo synthesis, cloning and subcloning of the miRNA sequences, constructing a plasmid with promoters and replication elements that are part of the plasmid containing the designed miRNA sequences all of which would be apparent to the skilled person.

The method for producing the AAV vector of the invention may comprise the steps of: a) culturing a host cell as herein defined above under conditions such that the AAV vector is produced; and, b) optionally, one or more of recovery, purification and formulation of the AAV vector.

The host cell is a host cell that is suitable for the production of AAV vectors. Accordingly, the host cell is a host cell that is amenable to in vitro culture, preferably at large scale. Host cells that are suitable for the production of AAV vectors are well-known in the art and will typically be a mammalian or an insect cell line. Mammalian cell lines for producing AAV vectors are selected from among any mammalian species, including, without limitation, cells such as A549, WEHI, 3T3, 10T1/2, BHK, MDCK, COS 1, COS 7, BSC 1, BSC 40, BMT 10, VERO, W138, HeLa, a HEK 293 cell (which express functional adenoviral E1), Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this invention; nor is the type of mammalian cell, i.e., fibroblast, hepatocyte, tumor cell. Mammalian cell lines for producing AAV vectors in particular include a broad range of HEK293 cell lines, of which the HEK293T cell line is preferred.

Insect cell lines for producing AAV vectors can be any cell line that is suitable for the production of heterologous proteins. Preferably the insect cell allows for replication of baculoviral vectors and can be maintained in culture, more preferably in suspended culture. In a preferred embodiment, the insect cell allows for replication of recombinant parvoviral vectors, including rAAV vectors. For example, the cell line used can be from Spodoptera frugiperda, Drosophila, or mosquito, e.g., Aedes albopictus derived cell lines. Preferred insect cells or cell lines are cells from the insect species which are susceptible to baculovirus infection, including e.g. S2 (CRL-1963, ATCC), Se301, SeIZD2109, SeUCR1, Sf9, Sf900+, Sf21, BTI-TN-5B11-4, MG-1, Tn368, HzAm1, Ha2302, Hz2E5, High Five (Invitrogen, CA, USA) and expresSF+® (U.S. Pat. No. 6,103,526; Protein Sciences Corp., CT, USA).

Thus, in some embodiments, the expression cassette or construct is an insect cell-compatible vector or a mammalian cell-compatible vector. An “mammalian cell-compatible vector” is understood to be a nucleic acid molecule capable of productive transformation or transfection of a mammalian cell or cell line. Mammalian cell-compatible vectors are well-known in the art. An “insect cell-compatible vector” is understood to be a nucleic acid molecule capable of productive transformation or transfection of an insect or insect cell. Exemplary insect cell-compatible vectors include plasmids, linear nucleic acid molecules, and recombinant viruses, such as baculoviruses.

Any vector can be employed as long as it is insect cell-compatible. The mammalian or insect cell-compatible vector may integrate into the cell's genome but the presence of the vector in the cell need not be permanent and transient episomal vectors are also included. The vectors can be introduced by any means known, for example by chemical treatment of the cells, electroporation, or infection.

The AAV in the supernatant can be recovered and/or purified using suitable techniques which are known to those of skill in the art. For example, monolith columns (e.g., in ion exchange, affinity or IMAC mode), chromatography (e.g., capture chromatography, fixed method chromatography, and expanded bed chromatography), centrifugation, filtration and precipitation, can be used for purification and concentration. These methods may be used alone or in combination. In one embodiment, capture chromatography methods, including column-based or membrane-based systems, are utilized in combination with filtration and precipitation. Suitable precipitation methods, e.g., utilizing polyethylene glycol (PEG) 8000 and NH₃SO₄, can be readily selected by one of skill in the art. Thereafter, the precipitate can be treated with benzonase and purified using suitable techniques. In addition, recovery may preferably comprises the step of affinity-purification of the (virions comprising the) recombinant parvoviral (rAAV) vector using an anti-AAV antibody, preferably an immobilised antibody. The anti-AAV antibody preferably is a monoclonal antibody. A particularly suitable antibody is a single chain camelid antibody or a fragment thereof as e.g. obtainable from camels or llamas (see e.g. Muyldermans, 2001, Biotechnol. 74: 277-302). The antibody for affinity-purification of rAAV preferably is an antibody that specifically binds an epitope on an AAV capsid protein, whereby preferably the epitope is an epitope that is present on capsid protein of more than one AAV serotype. E.g. the antibody may be raised or selected on the basis of specific binding to AAV2 capsid but at the same time also it may also specifically bind to AAV1, AAV3 and AAV5 capsids.

In general, suitable methods for producing an AAV vector according to the invention in mammalian or insect host cells, and means therefore (such as expression constructs for expression of AAV rep proteins), are described, for mammalian cells in: Clark et al. (1995, Hum. Gene Ther. 6, 1329-134), Gao et al. (1998, Hum. Gene Ther. 9, 2353-2362), Inoue and Russell (1998, J. Virol. 72, 7024-7031), Grimm et al. (1998, Hum. Gene Ther. 9, 2745-2760), Xiao et al. (1998, J. Virol. 72, 2224-2232) and Judd et al. (Mol Ther Nucleic Acids. 2012; 1: e54), and for insect cells in: Urabe et al. (2002, Hum. Gene Ther. 13:1935-1943), WO2007/046703, WO2007/148971, WO2009/014445, WO2009/104964, WO2011/122950, WO2013/036118, WO2015/137802, WO2019/016349 and in co-pending applications EP21177449.2, PCT/EP2021/058794 and PCT/EP2021/058798.

The methods for producing the pharmaceutical composition of the invention comprise any methods for producing pharmaceutical compositions, all of which would be apparent to the skilled person. Said methods generally comprise the combination of the nucleic acid or the AAV of the invention with excipients under specific conditions.

Another aspect of the invention relates to a kit comprising the nucleic acid of the invention, the expression cassette, the AAV vector of the invention, or the pharmaceutical composition of the invention, and an immunosuppressive compound.

In certain embodiments of the invention, the immunosuppressive compound may reduce and/or prevent an immune response induced by administration of the nucleic acid, the AAV vector, or the pharmaceutical composition of the invention.

In another aspect, the invention relates to a cell comprising the nucleic acid of the invention, the expression cassette, or the AAV of the invention, or a host cell.

In some embodiments, the cell of the invention is a prokaryote cell. In some specific embodiments, the cell of the invention is a bacterial cell. In some embodiments, the cell of the invention is a eukaryote cell. In some embodiments, the cell of the invention is a mammalian cell. In some embodiments, the cell of the invention is an insect cell.

The nucleic acid of the invention or the AAV vector of the invention may be delivered into the cell of the invention by any suitable methods, included but not limited to, transfection, transformation, electroporation, nucleofection, transduction and microinjection. For example, the expression cassette or nucleic acid of the invention may be comprised in a plasmid contained in bacteria. The expression cassette or nucleic acid of the invention may also be comprised in a production cell that produces e.g. a viral vector. Said expression cassette may also be provided in a baculovirus vector.

Further detail on host cells comprising an AAV vector according to the invention has been provide elsewhere in this application. Any mammalian cell may be infected by the AAV vector of the invention, including but not limited to a muscle cell, a liver cell, a nerve cell, a glial cell or an epithelial cell. In some preferred embodiments of the invention, the cell to be infected is a human cell.

In another aspect, the present invention relates to a method of treating or preventing a disorder, wherein the method comprises administering the nucleic acid of the invention or the AAV of the invention to a subject, thereby treating or preventing the disorder.

In certain embodiments, the disorder is caused by SNCA gene or a pathological human SNCA gene (duplication, triplication or mutations A30P, E46K, H50Q, G51D and A53T).

In certain embodiments, the disorder is PD, DLB, MSA, neuropsychiatric symptoms, motor symptoms of PD, cognitive impairment, sleep disturbances, autonomic disturbances, and/or olfactory disturbances or any atypical presentation mimicking other kinds of neurodegenerative brain diseases.

In another aspect, the invention relates to the nucleic acid of the invention, the expression cassette, or the AAV of the invention for use in the manufacture of a medicament for treatment of a disorder.

### BRIEF DESCRIPTION OF THE FIGURES

FIG. 1. Design strategy for guide sequences and miRNAs of the invention

FIG. 2. Design strategy of isoform-targeting guide sequences

FIG. 3. Dual Luciferase SNCA miRNA candidate selection: α-synuclein reporter lowering

FIG. 4. In vivo study in A53T KI rats (A. viral DNA (vDNA) levels; B. miSNCA expression levels; C. SNCA mRNA lowering in striatum)

FIG. 5. Dual Luciferase titration experiments: α-synuclein reporter lowering

FIG. 6. Design of candidate scaffolds

FIG. 7. Dual Luciferase assay results for candidate scaffolds

FIG. 8. Endogenous mRNA lowering results by the candidate scaffolds

FIG. 9. miRNA processing of the candidate scaffolds

FIG. 10. Motor behaviour improvement of C. elegans PD model treated with empty vector (EV), full-length SNCA RNAi (SNCA) or with the candidate scaffolds (miSNCA 5, 13, 15)

FIG. 11. SNCA mRNA lowering and α-synuclein protein lowering by full-length SNCA RNAi or the candidate scaffolds (miSNCA5; also named miRNA 5) in C. elegans PD model

FIG. 12. Small RNA sequencing results of C. elegans samples (Day 1 and Day 4) after treatment of the C. elegans at L1 stage (full length SNCA RNAi, miSNCA5 and miSNCA15)

FIG. 13. miRNA processing of the candidate scaffolds in vitro

FIG. 14. miRNA processing of the candidate scaffold 1 in vivo

FIG. 15. vDNA levels in striatum in in vivo Proof of Concept study in AAV-Syn rat model (Study 2).

FIG. 16. miSNCA levels in striatum in in vivo Proof of Concept study in AAV-Syn rat model (Study 2); (A) miSNCA2; (B) miSNCA5; (B) miSNCA15.

FIG. 17. Human SNCA mRNA levels in striatum in vivo Proof of Concept study in AAV-Syn rat model (Study 2).

FIG. 18. Human α-syn protein levels in striatum in in vivo Proof of Concept study in AAV-Syn rat model (Study 2).

FIG. 19. Dopamine transporter levels (assessed by ([125I]-RTI-121 autoradiography) in striatum in in vivo Proof of Concept study in AAV-Syn rat model (Study 2).

FIG. 20. Dopamine and dopamine metabolite levels (assessed by LC/MS) in striatum in in vivo Proof of Concept study in AAV-Syn rat model (Study 2).

FIG. 21. Motor behavior test (cylinder test, assessing paw use asymmetry) in in vivo Proof of Concept study in AAV-Syn rat model (Study 2). (A) At baseline (before any injections); (B) Day 56 post-treatment.

FIG. 22. Results of the in vivo Proof of Concept study in AAV-Syn rat model (Study 2), assessed by immunohistochemistry in (A) total TH; (B) total human α-syn; and (C) α-syn positive TH neurons in the substantia nigra.

### EXAMPLES

Within the experimental section, nucleic acids comprising SEQ ID NO. 95-98 and SEQ ID NO. 78-81 will be designated as “Scaffold 1”; the nucleic acids of the invention, comprising at least 3 RNAs, will be designated as “Scaffold 2”.

### Materials and Methods

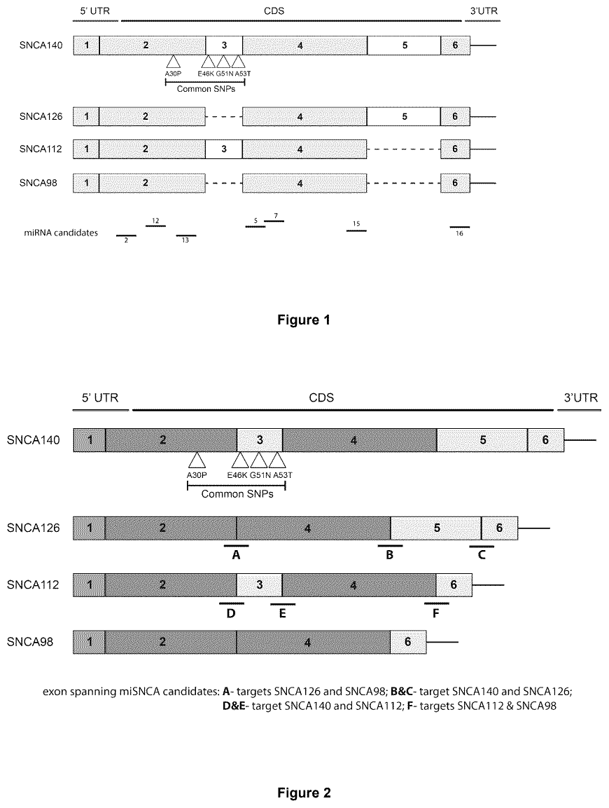

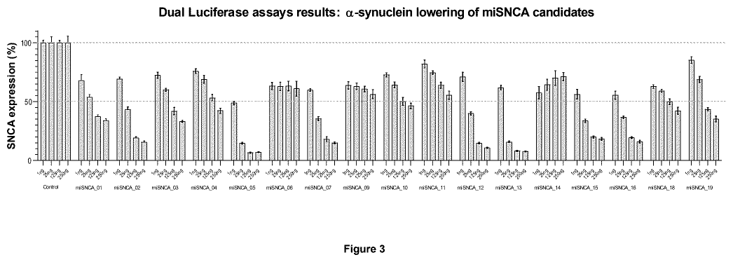

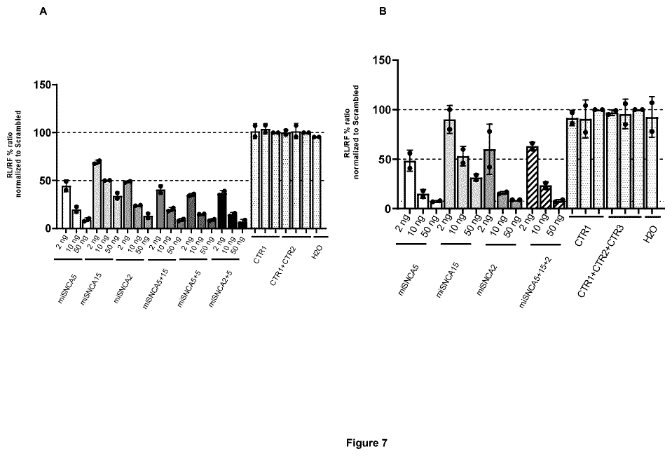

SNCA miRNA guide strand design. The miSNCAs (miRNA guide strands) were designed to target common RNA sequences of the most common SNCA mRNA variants; SNCA140, SNCA126, SNCA112 and SNCA98. For designing a miRNA that is targeting all of these variants, miRNAs in regions that are common with all of the major mRNA variants of SNCA were designed (FIG. 1). The target regions of the SNCA mRNA sequences are a portion of the exon 2, exon 4 and exon 6. The most common SNPs outside of these exons, namely A30P, was avoided to be included in the guide RNAs. Each of the conserved sequences was used to generate a number of different guide strands having 22 nts. Seventeen guides targeting SNCA were designed and incorporated into miR451 scaffold: miSNCA2 (SEQ ID NO.71), miSNCA5 (SEQ ID NO.72), miSNCA7 (SEQ ID NO.73), miSNCA12 (SEQ ID NO.74), miSNCA13 (SEQ ID NO.75), miSNCA15 (SEQ ID NO.76), miSNCA16 (SEQ ID NO.77), miSNCA1 (SEQ ID NO.82), miSNCA3 (SEQ ID NO.83), miSNCA4 (SEQ ID NO.84), miSNCA6 (SEQ ID NO.85), miSNCA9 (SEQ ID NO.86), miSNCA10 (SEQ ID NO.87), miSNCA11 (SEQ ID NO.88), miSNCA14 (SEQ ID NO.89), miSNCA18 (SEQ ID NO.90), and miSNCA19 (SEQ ID NO.91). These were tested in vitro for their efficacy to lower the concatenated SNCA reporter gene (SEQ ID NO.92) in Dual luciferase reporter plasmid in total and tested with Dual Luciferase assays (FIG. 3). Out of these seventeen miRNAs, seven of them showed potential to lower the SNCA mRNA levels in a dose-dependent manner (SEQ ID NO.71-77) (FIG. 5).

The miSNCA guides were selected based on the following criteria: the miRNA guide sequence should not include a stretch of >4 G, >4 C, >5 A and >5 T nt, a GC content between 30% and 70%, <4000 predicted off-target genes of the miRNA seed sequence for SNCA targeting guides and <5000 predicted off-target genes of the miRNA seed sequence for SNCA targeting guides by using siSPOTR analysis (https://sispotr.icts.uiowa.edu./sispotr/tools/lookup/evaluate.html) and pre-miRNA sequence folding energy between −44 and −55 kcal/mole. To generate a negative control, two scramble guides were selected for in vitro testing (miR144_miSCR(ANG1) (SEQ ID NO.93) and miR144_miSCR(ANG1)+miSCR(C90) (SEQ ID NO.94)).

The selected miSNCA guides meet the following criteria: conservation with monkey SNCA gene sequence (Macaca mulatta, NCBI accession number NC_041768.1), the miRNA guide sequence should not include a stretch of >4 G or >4 C nt, a GC content between 20% and 70%, a GC seed content between 40% and 70%, pre-miRNA sequence folding energy between −45 and −55 kcal/mole and no matching with endogenous miRNA seeds.

Guide sequences were incorporated into human pri-miRNA miR-451 scaffold sequences and the mFold program (http://unafold.ma.albany.edu/?q=mfold) was used with standard settings to determine whether the candidates are folded into the secondary structures.

The SNCA Scaffold 1) consists of the miR-144 hairpin/scaffold combined with one mir-451 downstream hairpin/scaffold. The SNCA Scaffold 2 consists of the miR-144 hairpin combined with two or more mir-451 downstream scaffold. Placement of the miR144 hairpin is always at the 5′ most compared to the miR451 hairpin sequence(s). Four SNCA Scaffolds 1 (SEQ ID NO. 95-98) were generated to target the SNCA mRNAs and four SNCA Scaffolds 2 were generated to target different or same region of the SNCA mRNA (SEQ ID NOs.78-81).

### Dual Reporter Luciferase Assay

For Dual Luciferase assays HEK293T cells (1×105 cells/well) were plated into 24-well tissue culture-treated plates in triplicates. The cells were co-transfected with a reporter plasmid (1 Ong) that is carrying full-length SNCA gene encoding for 140 amino acid long (full-length; SEQ ID NO. 39) α-synuclein protein and varying amounts (0.1-1-10-100 ng) of plasmid carrying miSNCA candidates (FIG. 1) using Lipofectamine 3000 (Thermo Fisher Scientific). The cells were then collected after two days of transfection and the cell samples were analyzed for Renilla Luciferase and Firefly Luciferase activity using the Dual Luciferase assay kit from Promega. The assays were performed in GloMax Luminescence reader. α-synuclein lowering was measured as a decrease in the RL/FL activity ratio. The experiments were repeated at least three times.

Dual Luciferase assays for targeting preferentially different mRNA variants of SNCA are carried out in a similar manner. HEK293T cells are co-transfected with a reporter plasmid (10 ng) carrying a single gene coding for a shorter mRNA variant coding for 126, 112 or 98 amino acids long α-synuclein isoforms and varying amounts (0.1-1-10-100 ng) of plasmid carrying miSNCA candidates (FIG. 2). The rest of the assay is carried out as described in the previous paragraph.

### Transfections and Endogenous α-Synuclein Lowering

To evaluate endogenous α-synuclein lowering by miSNCA candidates, HEK293T cells were used. For these assays the HEK293T cells (1 or 5×10⁵ cells/well) were plated into 24 or 6-well tissue culture-treated plates respectively. The cells were transfected with varying amounts (50-100-200-1000 ng) of plasmid carrying miSNCA candidates using Lipofectamine 2000 or Lipofectamine 3000 (Thermo Fisher Scientific). Each transfection was performed in triplicates. The cells were then collected two days after the transfections. The cell samples were analyzed for the mRNA levels of SNCA and α-synuclein protein levels. The experiments were repeated at least three times.

### Transfections and Endogenous α-Synuclein Lowering of the Isoforms

To evaluate endogenous α-synuclein lowering by miSNCA candidates targeting different isoforms, HEK293T cells are used. For these assays the HEK293T cells (1 or 5×105 cells/well) are plated into 24 or 6-well tissue culture-treated plates respectively. The cells are transfected with varying amounts (50-100-200-1000 ng) of plasmid carrying miSNCA candidates using Lipofectamine 2000 or Lipofectamine 3000 (Thermo Fisher Scientific). Each transfection is performed in triplicates. The cells are then collected two days after the transfections. The cell samples are analysed for the mRNA levels of mRNA splicing variants of SNCA using RT qPCR with exon spanning primer sets as described in McLean et al., 2012 Mol. and Cell. Neuroscience, 49(2):230-239. Furthermore, from those cell samples, α-synuclein isoform protein levels are analysed by Western blots using antibodies that are targeting different isoforms or antibodies recognizing an epitope present in all isoforms (each isoform can then be distinguished by its molecular weight). The experiments are repeated at least three times.

### DNA Constructs for Baculovirus Seed Generation

The expression cassettes carrying candidate miSNCA constructs were subcloned into pVD1746 (SEQ ID NO. 40), pVD1747 (SEQ ID NO. 41), pVD1748 (SEQ ID NO. 42), pVD1749 (SEQ ID NO. 43), pVD1750 (SEQ ID NO. 44), pVD1751 (SEQ ID NO. 45), and pVD1752 (SEQ ID NO. 46), containing ITR regions for AAV5 and AAV6 packaging.

The pVD plasmids comprising Scaffold 1 constructs carry a constitutive promoter P1 (SEQ ID NO. 47), followed by the sequence encoding the RNAs of the invention and a hGH polyA sequence (SEQ ID NO. 48). pVD plasmids comprising Scaffold 2 constructs carry a short CAG promoter, followed by the sequence encoding the RNAs of the invention and hGH polyA sequence (SEQ ID NO. 48).

### AAV5 Vectors (HEK Material)

AAV production by co-transfection of HEK 293T cells with a transfer vector and packaging plasmid(s) for the AAV5 serotype. Purification of the AAV was done via affinity chromatography & iodixanol gradient ultracentrifugation. After formulation and concentration, the titer of the purified AAV was determined using QPCR.

### AAV5 Vectors (Baculovirus Material)

Recombinant AAV5 harbouring the expression cassettes were produced by infecting SF+ insect cells (Protein Sciences Corporation, Meriden, Connecticut, USA) with two Baculoviruses, encoding Rep, Cap and Transgene. Following standard protein purification procedures on a fast protein liquid chromatography system (AKTA Explorer, GE 30 Healthcare) using AVB sepharose (GE Healthcare) the titer of the purified AAV was determined using QPCR.

### AAV6 Vectors (HEK Material)

Recombinant AAV6 harbouring the expression cassettes were produced by co-transfection of HEK293T cells with packaging plasmids encoding Rep, Cap and the Transgene. Following standard protein purification procedures on a fast protein liquid chromatography system (AKTA Explorer, GE 30 Healthcare) using AVB sepharose (GE Healthcare) the titer of the purified AAV was determined using QPCR.

### In Vitro Models and Transduction Assays

To measure the effects of AAV5-miSNCA Scaffold1/2 on human α-synuclein mRNA and protein levels, control non-patient-derived iPSC-derived forebrain neurons, patient-derived iPSC-derived forebrain neurons and Lund human mesencephalic (LUHMES) derived DA neurons are used. IPSC cell lines (Table 1) are obtained from NINDS RUCDR repository and LUHMES cells are obtained from ATCC.

TABLE 2
iPSC cell lines and controls

| | Subject | Cell Line | Gene | |
|---|---|---|---|---|
| Short name | ID | ID | affected | Genotype |
| A53T | NDS00188 | ND50050 | SNCA | ALA53THR |
| Isogenic control | NDS00188 | ND50085 | SNCA | Isogenic (edited) |
| Edited | NDS00188 | ND50086 | SNCA | Heterozygous |
| (heterozygous) | | | | (edited) |
| SNCA3X | NDS00201 | ND50040 | SNCA | SNCA triplication |
| Control | NDS00159 | ND41865 | none | Non-patient |
| | | | | control |

Differentiation of iPSC cells into forebrain neurons is done using Forebrain Neurons Differentiation kit from StemCell Technologies. The LUHMES cells were differentiated into DA neurons using the protocol described in Harischandra et al., 2020 BBA Mol. Basis of Disease 1866:165533.

The above-mentioned in vitro cell models are transduced using either HEK cell- or Baculovirus produced AAV5-miSNCA Scaffold 1 and AAV5-miSNCA Scaffold 2 (or HEK293T-cell produced AAV6-miSNCA Scaffold 1/2) candidates at various multiplicity of infection (MOI) of the virus. The cells are plated into PDL-Laminin, or PLO-Laminin coated 6-well plates at 5×10⁵ cells/well. After 3-4 days of passaging, the cells are transduced at MOI of 10³, 10⁴, 10⁵ and 10⁶/cell. The cells are then collected at 7-15 days after the transduction. The cell samples are used for RNA and DNA isolation to determine vector DNA levels, miSNCA expression, SNCA mRNA and α-syn protein expression.

### RNA Isolation and Small RNA Sequencing Using Next-Generation Sequencing (NGS)

A. From HEK293T Cells Transfected by miSNCA Scaffolds 1 and 2 Candidates

Total RNA was isolated from the HEK293T cells that were transfected with 10 ng of miSNCA scaffold 1 or 2 containing plasmids (with 10 ng plasmid for of 1×10⁵/cell) using Direct-zol RNA Microprep Kits (ZymoResearch) with the DNAse treatment step. Total RNA was checked for quality and quantified using Nanodrop. The samples were then sent for small RNA sequencing to GenomeScan BV (Leiden, Netherlands). Small RNA sequencing was performed by GenomeScan using the NebNext small RNA library prep method including BluePippin size selection of the final library combined with Illumina NovaSeq6000 PE150 sequencing. The data was analysed using CLC Genomics v20 Suit (Qiagen). The trimmed small RNA reads were mapped against the human database of micro-RNAs (miRbase v22) and annotated in parallel to mapping and annotation to custom database containing the synthetic (pre-) miRNA sequences. The quantification of the miSNCA, and miR-144 A>T (guide and passenger strands when relevant) sequences was expressed as the number of counts/sequence versus the total number of small RNA counts per sample. The processing of the miSNCA candidates was also investigated by alignment of the reads to the miRNA guide sequences and quantified as the number of counts for one isomiR sequence versus the total number of isomiR counts per sample.

B. From Neuronal Cell Models Transduced with AAV-miSNCA Scaffolds 1 and 2 Candidates

RNA is isolated from the AAV5- or AAV6-Scaffold 1 or 2 (either HEK-cell or Baculovirus produced) transduced cells (DA neurons, forebrain neurons and/or LUHMES derived DA neurons) using Zymogen RNA isolation kit. The RNA quality is tested using Bioanalyzer and quantified using Nanodrop. Then the samples are sent for small RNA sequencing to GenomeScan BV (Leiden, Netherlands) using next generation sequencing methods. The data is analyzed using CLC Genomics Suit (Qiagen), to extract information about the expression values of the miSNCA candidates and also to evaluate the processing of the miSNCA candidates that are expressed from AAV5 or AAV6 packaged Scaffold 1 and 2 candidates.

### Small RNA Sequencing (NGS) Data Analysis

Analysis of the data obtained from neurons transduced with AAV5-Scaffold1/Scaffold 2 or AAV6-Scaffold1/Scaffold 2 miSNCAs is carried out using CLC Genomics Workbench 21 suit. The trimmed small RNA sequence reads are counted and annotated using miRbase database. The miSNCA molecules are annotated by aligning the pri-miRNA sequence against these small RNA library. The expression values of the miSNCA candidates are expressed as the number of counts of miSNCA candidate counts versus the whole annotated small RNA counts. The mostly expressed miSNCA molecules are analyzed by looking at the relative counts of the various sizes of the miSNCA aligning the pre-miSNCA to the small RNA library and using the RNA counts obtained from there.

### RNA Sequencing (NGS) and Off Target Analysis

RNA sequencing is done on the samples collected from transduced forebrain neurons or differentiated LUHMES cells.

### A. Data Processing and Quality Control

Paired-end RNA-seq data are generated for all samples using the Illumina NovaSeq 6000. Alignment of reads from all samples to human genome is performed using STAR aligner. Raw expression data are evaluated using several automated outlier tests including sum of Euclidean distance, Hoeffding's D-statistic, mean Pearson correlation and the Kolmogorov-Smirnov test statistic.

Expression data is generated. Normalisation is carried out using trimmed mean normalization and data were transformed with voom (https://genomebiology.biomedcentral.com/articles/10.1186/gb-2014-15-2-r29).

Principal component analysis (PCA) and hierarchical clustering are performed to identify clustering of samples.

b. Differential Expression

Ten statistical contrasts are performed in order to determine significantly differentially expressed genes (DEGs) between all sample groups. Significant DEGs are determined using a statistical threshold corrected for multiple testing using the false discovery rate (FDR) adjustment.

Vector DNA Isolation and Quantification from Cells and Animal Tissues

DNA extraction was performed using AllPrep DNA/RNA Mini Kit (Qiagen) and following the manufacturer's instructions. Vector genome copies were quantified by using TaqMan qPCR assay (Thermo Fisher Scientific) with primers against the polyA region of the vector. The quantification (GC/ug DNA) was done using linearized pVD plasmid and making a standard curve with varying amounts of this linearized plasmid. The standard curve created this way was used to calculate the vector DNA copy number from the DNA isolated from cells transduced with AAV5-miSNCA Scaffold 1 and AAV5-miSNCA Scaffold 2.

RNA and Protein Isolation from Transfected HEK Cells and Quantification of mRNA of SNCA and α-Synuclein Protein Levels

For the RNA Isolation, the Direct-zol™ RNA Miniprep (Catalog no. R2050) was used. TRizol was applied to the snap frozen cell pellets to lyse them. The cDNA syntheses were performed using the Maxima First Strand cDNA Synthesis Kit (Thermo Fisher Scientific) for RT-qPCR.

For protein isolations, RIPA buffer (Sigma) containing PhosSTOP phosphatase inhibitor (Roche) and EDTA-free protease inhibitor (Roche) was used. For extraction of the protein the buffer was added into the cell pellet and the cells were agitated at 4 C, 400 rpm for 30 minutes. The cell extract was then centrifuged at top speed. The clarified supernatant was used for α-synuclein and total protein measurement i.e. HTRF and BCA assays.

For the detection of mSNCA levels SYBR Green based RT-qPCR assay was used using a set of primers designed against SNCA (Table 3). The results were displayed as fold change using the ΔΔCycle threshold (ΔΔCt) of the treated sample against the untreated sample normalized to the mean expression of household genes (Table 3).

TABLE 3
Primer sequences for RT-qPCR of HEK cells.

| SEQ ID | | Type of | | |
|---|---|---|---|---|
| NO. | Primer | gene | Forward primer | Reverse primer |
| 49 & 50 | SNCA1 | transgene | AAATGTTGGAGGAG | TCCAGAATTCCTTCCTGT |
| | | | CAGTGG (SEQ ID | GG (SEQ ID NO. 50) |
| | | | NO. 49) | |
| 51 & 52 | SNCA2 | transgene | ATGTTGGAGGAGCA | TGTCAGGATCCACAGGCA |
| | | | GTGGTG (SEQ ID | TA (SEQ ID NO. 52) |
| | | | NO. 51) | |
| 53 & 54 | GFP | transgene | AGCAAAGACCCCAA | GCGGCGGTCACGAACTC |
| | | | C GAGAA (SEQ ID | (SEQ ID NO. 54) |
| | | | NO.53) | |
| 55 & 56 | B2M | House- | GCCATCCACCGGAG | GGTGGAACTGAGACACGT |
| | | keeping | AATG (SEQ ID NO. | AGCA (SEQ ID NO. 56) |
| | | gene | 55) | |
| 57 & 58 | GAPDH | House- | TGCCCCCATGTTTG | GCTGACAATCTTGAGGGA |
| | | keeping | TGATG (SEQ ID NO. | GTTGT (SEQ ID NO. 58) |
| | | gene | 57) | |
| 59 & 60 | ACTB | House- | AGCGTGGCTACAGC | AAGTCTAGGGCAACATAG |
| | | keeping | TTCACC (SEQ ID NO. | CACAGC (SEQ ID NO. 60) |
| | | gene | 59) | |
| 61 & 62 | HPRT | House- | GCGAAAGTGGAAAA | GCCACATCAACAGGACTC |
| | | keeping | GCCAAGT (SEQ ID | TTGTA (SEQ ID NO. 62) |
| | | gene | NO. 61) | |

For the detection of α-synuclein protein levels the total α-syn HTRF kit (Cisbio) was used. The HTRF measurements were then normalized by total protein added to the HTRF assay. Total protein measurements were done using a bicinchoninic acid assay (BCA Protein assay kit; Pierce™). The HTRF results were given as the HTRF ratio/μg total protein.

RNA Isolation and Quantification of miSNCA Candidates, GFP mRNA and SNCA mRNA from Animal Tissues

Tissue was homogenized using the Tissue Lyser system (Qiagen) and AllPrep DNA/RNA Mini kit (Qiagen) following the manufacturer's instructions. DNA and RNA quantity and integrity were determined by Nanodrop and Bioanalyzer.

For miSNCA expression, the following protocol was used: Total RNA was isolated using AllPrep DNA/RNA Micro kit (Qiagen). RT-qPCR using a Taqman stem-loop-miRNA assay (Thermo Fisher) designed for detecting 28 nts miSNCA2, 23 nts miSNCA5 and 22 nts miSNCA15 the level of miRNA expression was used. The expression levels were expressed as miRNA molecules/ug total RNA.

For mRNA expression, the following protocol was used: Total RNA was isolated using AllPrep DNA/RNA Micro kit (Qiagen). A Taqman assay for RT-qPCR was used to measure SNCA and GFP mRNA expression. The genes that were used as house-keeping genes are; ACTB, B2M, GAPDH, HPRT. Primer sequences shown in Table 4.

TABLE 4
Primer and probe sequences for
RT-qPCR from animal tissues.

| SEQ ID | | Type of | |
|---|---|---|---|
| NO. | Gene | gene | Primer Sequence |
| 49 | SNCA1 | transgene | 5′-AAATGTTGGAG |
| | | | GAGCAGTGG-3′ |
| 50 | SNCA1 | transgene | 3′-TCCAGAATTC |
| | | | CTTCCTGTGG-5′ |
| 51 | SNCA2 | transgene | 5′-ATGTTGGAGGA |
| | | | GCAGTGGTG-3′ |
| 52 | SNCA2 | transgene | 3′-TGTCAGGATC |
| | | | CACAGGCATA-5′ |
| SEQ ID | | Type of | |
| NO. | Gene | gene | Probe Sequence |
| 63 | SNCA1 | transgene | 5′-AGCAGGGAGCA |
| | | | TTGCAGCAGC-3′ |
| 64 | SNCA2 | transgene | 5′-CAGCAGCCACT |
| | | | GGCTTTGTCAAA-3′ |

LC-MS/MS from Animal Tissues

Striatal tissue samples were sent on dry-ice to the Vanderbilt Neurochemistry Core Facility (Nashville, TN, USA) for determination of catecholamine levels and data returned blinded for analysis.

Tissue Extraction. Brain sections were homogenized, using a tissue dismembrator, in 100-750 μl of 0.1 M TCA containing 10-2 M sodium acetate, 10-4 M EDTA, and 7.5% methanol (pH 3.8). 10 μl of homogenate was removed for measurement of protein concentration. The samples were then spun in a microcentrifuge at 10,000 g for 20 minutes at 4° C. Supernatant was transferred to a new microcentrifuge tube for biogenic amine analysis.

Biogenic Amine Analysis. Dopamine, HVA, and DOPAC levels were determined by a highly sensitive and specific liquid chromatography/mass spectrometry (LC-MS/MS) methodology following derivatization of analytes with benzoyl chloride (BZC). 5 μl of supernatant was treated with 10 μl each of 500 mM NaCO3 (aq) and 2% BZC in acetonitrile. After four minutes, the reaction was stopped by the addition of 10 μl internal standard solution (in 20% acetonitrile containing 3% sulfuric acid) containing 200 μg of each 13C6-derivatized dopamine-d4, HVA, and DOPAC. Liquid Chromatography was performed on a 2.0×50 mm, 1.7 μm particle Acquity BEH C18 column (Waters Corporation, Milford, MA, USA) using a Waters Acquity UPLC. Mobile phase A was 0.15% aqueous formic acid and mobile phase B was acetonitrile. Samples were separated by a gradient of 98-5% of mobile phase A over 11 min at a flow rate of 600 μl/min prior to delivery to a SCIEX 6500+ QTrap mass spectrometer (AB Sciex, Framingham, MA, USA). The following MRM transitions were monitored for quantitative purposes: 466 to 105, BZC-dopamine; 488 to 111, 13C6-BZC-dopamine-d4; 304 to 150, BZC-HVA; 310 to 111, 13C6-BZC-HVA; 394 to 105, BZC-DOPAC; 406 to 111, 13C6-BZC-DOPAC. Automated peak integration was performed using SCIEX Multiquant software version 3.0.2. All peaks were visually inspected to ensure proper integration. Levels of dopamine, HVA, and DOPAC in samples were calculated using calibration curves constructed on the basis of peak area ratio (Panalyte/PI.S.) versus concentrations of internal standard by linear regression. Levels were normalised to protein concentration in the tissue extract.

Protein assay. Protein concentration in tissue homogenates was determined using the Pierce™ BCA Protein Assay Kit (Thermo Fisher Scientific, Waltham, MA USA) as described in the provided kit instructions. Absorbance was measured using a POLARstar Omega plate reader (BMG LABTECH, Offenburg, Germany).

ELISA for Transene-Derived Human α-syn

Dissected striatal tissue from fresh frozen cryosections of all animals was homogenised in a lysis buffer containing protease and phosphatase inhibitors (Roche: 11836153001). Samples were agitated at 4° C. for 30 minutes followed by centrifugation (135000 rpm for 10 minutes at 4° C.) to produce supernatant. Using a 1:500 dilution for a concentration of 0.001 mg/ml, a portion of supernatant was used to determine total protein levels (BCA assay, Pierce, Rockford, IL). Another portion of supernatant underwent ELISA procedures according to the manufacturer's instructions (BioLegend: 844101). The samples were analysed using CLARIOstar systems quantifying the luminescent counts relative to the amount of aSyn. Levels of α-Syn were expressed as pg/mg total protein (Pierce™ BCA Protein Assay Kit, Thermo Fisher Scientific, Waltham, MA USA).

### Dopamine Transporter (DAT) Binding

The levels of striatal DAT were be assessed by [125I]-RTI-121 binding autoradiography in cryostat cut sections prepared from 20 μm fresh-frozen tissue. Briefly, thawed slides were placed in binding buffer (2×15 min, room temperature) containing 50 mM Tris, 120 mM NaCl and 5 mM KCl. Sections were then placed in the same buffer containing 50 pM [125I]-RTI-121 (Perkin-Elmer, specific activity 2200 Ci/μmol) for 120 min at 25° C. to determine total binding. Non-specific binding was defined as that observed in the presence of 100 μM GBR 12909 (Tocris Bioscience). All slides were then washed (4×15 min) in ice-cold binding buffer, rinsed in ice-cold distilled water and air-dried. Together with [125I]-microscale standards (Amersham), slides were then apposed to autoradiographic film (Kodak) and left for ˜7 days at room temperature before developing. Autoradiograms were then analysed using MCID software (Image Research Inc, Ontario, Canada). Densitometric analysis of 3 striata from each animal was carried out whereby a reference curve of c.p.m. versus optical density was calculated from β-emitting [14C] micro-scale standards and used to quantify the intensity of signal as nCi/g. Background intensity was subtracted from each reading. Data were then expressed as mean±s.e.m. signal intensity for each treatment group. Non-specific binding was calculated in the same way and subtracted from the total to give specific binding. Non-specific binding was typically found to account for <1% of total binding.

### Immunofluorescence and Stereology

Immunofluorescence: Brains were sectioned frozen in the coronal plane at a thickness of 40 μm on a freezing sliding microtome (Leica Microsystems Inc., Richmond Hill, ON) and 6 series of sections were stored in cryoprotectant (30% glycerol, 30% ethoxyethanol, 40% PBS). Using a single series of midbrain sections, double label immunofluorescence was performed to reveal hemagglutinin (HA)-tagged human aSyn and tyrosine hydroxylase (TH). Briefly, on free floating sections, levels and distribution of TH (sheep anti-TH, 1:1000, Pel Freez, P60101; secondary antibody, Alexa fluor donkey anti-sheep, Fisher Scientific, A21099, 1:500) and HA (rabbit anti-HA, 1:1000; Abcam, AB9110; Alexa Fluor donkey anti-rabbit, 1:500, Fisher Scientific, A21206, 1:500) was evaluated by double label immunofluorescence.

Stereology: Estimates of TH+ve neuronal number with and without human α-syn co-localization within the substantia nigra pars compacta (SNc) was performed using Stereo Investigator software (MBF Bioscience, Williston, VT) according to stereologic principles. Seven or eight sections, each separated by 240 μm from the anterior to the posterior SN, was used for counting each case. Stereology was performed using a Zeiss microscope (Axiolmager M2 with Apotome, Carl Zeiss, Canada) coupled to a monochrome digital camera for visualization of tissue sections. The total number of TH+ve neurons with and without human α-syn inclusions was estimated from coded slides using the optical fractionator method. For each tissue section analyzed, section thickness was assessed empirically and guard zones of ˜2 μm thickness were used at the top and bottom of each section. The SNc was outlined under low magnification (5×) and TH+ve neurons counted under 40× magnification. Stereological parameters were empirically determined (i.e. grid size, counting frame size and dissector height) using Stereo Investigator software (MicroBrightfield, VT, USA). The acceptable coefficient of error (CE) was calculated according to the procedure of West and colleagues, known as the Gunderson CE (m=1). Gunderson values<0.10 were accepted.

The results of the counting stereology produced absolute numbers of TH+ve neurons in the SNc to assess neuroprotection. The number of those TH+ve neurons remaining that contain reactivity for human alpha-synculein were also produced to provide an indication of the number of human α-syn expressing TH+ve neurons. A ratio of TH+ve/synuclein+ve: TH+ve/synuclein-ve was then calculated.

### Study Design In Vivo Studies

### Study 1. Mechanism of Action Study in α-Synuclein KI Rats

In this study, the mechanism of action of two AAV-miSNCA candidates was evaluated in human α-synuclein KI rats. A total of three treatment groups with N=3 animals per group (total N=9, Envigo, USA) were used. On Day 1, all animals received a 3×3 ul unilateral injection of AAV5 into the striatum. The contralateral side served as the control and was injected with formulation buffer in the same manner as the AAV5 injections. Groups are indicated in Table 5.

TABLE 5
Groups of in vivo study 1.

| | | | | Terminal | |
|---|---|---|---|---|---|
| | | | | procedures - | |
| | Ipsilateral | Contralateral | Rat | brain | |
| Group | (AAV5) | (Buffer) | strain | samples | N |
| 1 | AAV5-miSCR | formulation buffer | Rat-KI | D 43 | 3 |
| 2 | AAV5-miSNCA5 | formulation buffer | Rat-KI | D 43 | 3 |
| 3 | AAV5-miSNCA15 | formulation buffer | Rat-KI | D 43 | 3 |
| 4 | AAV5-miSNCA5 + 15 | formulation buffer | Rat-KI | D 43 | 3 |

AAV5 titers = 2.5 × 10Exp11 gc/brain, 3 sites intra-striatal; Rat-KI = Envigo human α-synuclein_knock-in rat.

Striatal stereotaxic injection coordinates were site 1: +1.3 mm AP, −/+2.8 ML, −4.5 DV; site 2: +0.2 mm AP, −/+3.0 ML, −5.0 DV; site 3: site 2: −0.6 mm AP, −/+4.0 ML, −5.5 DV; with the toothbar set at −3.3. Viral vectors were administered at a rate of 0.5 ul/min and a 5 min wait time allowed after each injection.

On Day 43, all rats received an overdose of isoflurane and transcardially perfused with ice-cold 0.9% saline. Brains were then removed as rapidly as possible and left and right hemispheres were divided. In all animals from each group, the following regions were freshly dissected, separately for left and right hemispheres, frozen on dry ice and stored at −80° C. for molecular analyses: prefrontal cortex, striatum, hippocampus, hypothalamus, thalamus, posterior cortex, cerebellum, ventral midbrain and brainstem.

### Study 2. Proof of Concept Study in AAV-α-Synuclein Rat Model

This study is designed to assess the ability of two miRNAs targeting SNCA mRNA (encoding α-synuclein) to protect dopaminergic function in the AAV1/2-hA53T-aSyn rat model of Parkinson's disease. On day 1 (D1), AAV1/2 human A53T α-synuclein (AAV1/2-hA53T-α-Syn), in combination with one of 4 other AAV5 vectors, is administered unilaterally into the right substantia nigra according to stereotaxic techniques. Behavioural assessment is conducted in the cylinder test, to assess forelimb asymmetry, prior to surgery (baseline, D-3) and on D14, D21, D42 and D56 (2, 3, 6 and 8 weeks following AAV administration). Groups are indicated in Table 6.

TABLE 6
Groups of in vivo study 2.

| | Viral vector -1 | Viral vector -2 | |
|---|---|---|---|
| Group | intra-SN, 2 ul volume | | N |
| 1 | D 1: AAV1/2-EV | D 14: AAV5-unrelated | 12 |
| | | miR | |
| 2 | D 1: AAV1/2-hA53T- | D 14: AAV5-unrelated | 12 |
| | aSyn | miR (for scaffold 1) | |
| 3 | D 1: AAV1/2-hA53T- | D 14: AAV5-unrelated | 12 |
| | aSyn | miR (for Scaffold 2) | |
| 4 | D 1: AAV1/2-hA53T- | D 14: AAV5-miSNCA2 | 12 |
| | aSyn | | |
| 5 | D 1: AAV1/2-hA53T- | D 14: AAV5-miSNCA5 | 12 |
| | aSyn | | |
| 6 | D 1: AAV1/2-hA53T- | D 14: AAV5-miSNCA15 | 12 |
| | aSyn | | |
| 7 | D 1: AAV1/2-hA53T- | D 14: AAV5- | 12 |
| | aSyn | miSNCA5 + 15 low dose | |
| 8 | D 1: AAV1/2-hA53T- | D 14: AAV5- | 12 |
| | aSyn | miSNCA5 + 15 mid dose | |
| 9 | D 1: AAV1/2-hA53T- | D 14: AAV5- | 12 |
| | aSyn | miSNCA5 + 15 high dose | |

AAV1/2-hA53T-aSyn = AAV1/2 human A53T α-synuclein, 3 x; AAV1/2-EV = empty α-Syn vector; D 15, viral vector 2; Group 1 to 6, and Group 9, 3 × 10¹³ gc/ml; Group 7, 6 × 10¹² gc/ml,; Group 8, 1.2 × 10¹² gc/ml,; SN = substantia nigra; D = day. All groups are sequential injections of AAVs on D 1 and D 14.

On D57, animals were sacrificed for post-mortem assessments. Blood, samples were collected, processed and stored as required.

Primary endpoints of the study included: assessment of forelimb asymmetry (via cylinder test); quantification of striatal dopamine and metabolite levels (via LC-MS/MS); quantification of dopamine transporter (via autoradiography); and quantification of striatal transgene-derived aSyn levels (via ELISA).

Optional endpoints included: quantification of tyrosine hydroxylase positive (TH+ve) cells in the substantia nigra with and without co-expression with human aSyn (via double label immunofluorescence).

Sacrifice and sample collection were done as follows: animals were deeply anaesthetized with isoflurane and then sacrificed via exsanguination by way of transcardial perfusion with ice-cold 0.9% saline containing 0.2% heparin. Brains were placed, ventral up, into an ice-cold stainless steel rat brain matrix and were first cut in the coronal plane at the level of the hypothalamus. The rostral portion of the brain, including the entire striatum, was immediately frozen in isopentane chilled to −42° C. and later sectioned for DAT autoradiography and dissected for the quantification of levels of dopamine and metabolites of dopamine (HVA and DOPAC) by LC-MS/MS and levels of human aSyn by ELISA. Tissues were stored in a locked freezer at −80° C. Additional regions of interest (including additional striatal dissections) were collected according to table 11 for molecular assays.

TABLE 9
Brain regions of interest and corresponding
assays and sample handling.

| Region of | | | |
|---|---|---|---|
| interest | Assay | Buffers used | Format |
| striatum | autoradiography | None, cryosectioned | on-slide |
| striatum | LCMS | See LCMS method | supernatant |
| | | below | |
| striatum | ELISA | RIPA, protease and | share |
| | | phosphatase inhibitors | aliquots |
| striatum | TR-FRET | RIPA, protease and | between |
| | | phosphatase inhibitors | assays |
| striatum | Q-PCR (DNA) | None, cryosectioned | In tube |
| | RT-QPCR | and dissected | |
| | (miRNA, RNA) | | |

The remainder of the caudal portion of the brain, including the mesencephalon, was immersed in 4% paraformaldehyde (PFA) for 48 hours for fixation followed by cryoprotection in graded sucrose solutions (15 to 30% sucrose). Tissue prepared in this manner was used for quantification of dopamine neuron numbers in the SNc via immunohistochemistry of tyrosine hydroxylase and unbiased stereology.

Study 3. Phenotypic Rescue of Motor Phenotype in C. elegans PD Model

In this study, the effect of expression of miSNCA candidates on phenotypic rescue of altered motor behaviour in a C. elegans PD model (OW40; van Ham et a/2008 PLoS Genet 4(3): el 000027) was studied. In this model α-synuclein is overexpressed in the body wall muscle of C. elegans. This α-synuclein overexpression causes the slowing of the movement of the worms when compared to the control worms. We studied the effect of decreasing SNCA mRNA levels thereby decreasing the α-synuclein protein levels by RNAi using the full length SNCA gene or our miSNCA constructs. The double stranded RNA with one of these constructs were introduced to the organism by feeding. The C. elegans OW40 worms were fed by E. coli that were overexpressing either the empty T444T plasmid as negative control or the full-length SNCA gene or one of our miSNCA candidates (miSNCA5, miSNCA13 or miSNCA15) at different stages of their life; Larval stage 1 (L1), Larval Stage 4 (L4) and Day 1 of their adulthood. The treatment experiments were repeated at 25° C. and at 15° C. degrees. Following treatment on day 1, day 4 and day 8 of their adulthood, the worms were video tracked using a high throughput tracking set up to measure their movement (speed as μm/s) (Perni et al 2018 Journal of Neuroscience Methods, 306:57-67).

Additional read-outs were RT-qPCR of the SNCA mRNA and α-synuclein protein levels using western blot analysis. The primers used for RT-qPCR of the SNCA mRNA is given in Table 7.

TABLE 7
qPCR primers for SNCA mRNA determination
in C. elegans samples

| SEQ | | | |
|---|---|---|---|
| ID NO. | Gene | Type of gene | Primer |
| 65 | SNCA1 | transgene | 5′-GACTTTCAAAGGC |
| | | | CAAGGAG-3′ |
| 66 | SNCA1 | transgene | 3′-GGAGCCTACATAG |
| | | | AGAACAC-5′ |
| 67 | SNCA2 | transgene | 5′-ATTGCAGCAGCCA |
| | | | CTGGCTTTG-3′ |
| 68 | SNCA2 | transgene | 5′-GGCTTCAGGTTC |
| | | | GTAGTCTTG-3′ |
| 69 | PMP3 | House-keeping | 5′-CACTCATCTCTAT |
| | | gene | GACGACGTTTC-3′ |
| 70 | PMP3 | House-keeping | 3′-CACCGTCGAGAA |
| | | gene | GCTGTAGA-5′ |

For detection of α-synuclein protein levels western blot analysis was used. For this purpose the proteins were extracted using RIPA buffer and Tissuelyser (Qiagen). Similar protein amounts from different treatment conditions were loaded onto the SDS PAGE and western blot was performed using anti human α-synuclein antibody (Table 8) for detecting the α-synuclein levels. For normalization tubulin was used and tubulin was detected using anti tubulin antibody (Table 8).

TABLE 8
Antibodies used for Western Blot analysis of C. elegans samples

| Antibody name | Catalog # | Type of protein |
|---|---|---|
| α-synuclein | 18-0215 (Invitrogen) | Target protein |
| Tubulin | T6074 (sigma) | Housekeeping protein for |
| | | normalization |

### Study 4. Study in PFF Rat Model

In this study the propagation of α-synuclein (α-Syn) pathology in transgenic mice (Line 83, or Line 20 or line 61) following administration of α-Syn-preformed fibrils (PFFs) to the striatum is evaluated. The effect of AAV5-Scaffold 1 or AAV5-Scaffold 2 in reducing the propagation of α-Syn pathology is assessed.

Animals receive a stereotaxic injection of α-Syn-PFF (mouse or human based) or respective monomer as control, in combination with control AAV (non-targeting miRNA) or different doses of AAV5-miSNCA.

At different time points after administration of α-Syn with or without AAV, animals sacrificed via transcardial perfusion with ice-cold 0.9% saline followed by 4% PFA. Brains are then removed and post-fixed in 4% paraformaldehyde (overnight) and cryoprotected in sucrose solutions. Forebrain and midbrain are then cut on a freezing sliding microtome for histological procedures. Qualitative assessment of pSer129 α-Syn expression for extent of pathological α-Syn spread in the forebrain (prefrontal cingulate cortex, insular cortex, striatum) and midbrain (substantia nigra; SN) is assessed by immunohistochemistry.

### Study 5. Dose-Response Study in Rodent Model of Parkinson's Disease

In this study, several doses of the most potent AAV5-miSNCA candidates determined in previous experiments, are tested in a relevant rodent (rat or mouse) Parkinson's disease model (transgenic line). Molecular, histological and motor readouts are used to determine a dose-response relationship between AAV5-miSNCA and phenotypic rescue.

### Results

### In Vitro Experiments

### Example 1. In Vitro Silencing Efficacy of Artificial miSNCA Constructs

To evaluate the miSNCA knockdown efficacy of the miSNCA constructs in vitro, HEK293T cells were co-transfected with Renilla luciferase reporters encoding the SNCA gene. The Firefly luciferase (FL) gene was expressed from the same reporter vector and served as an internal control to correct for transfection efficiency. In the first screening HEK cells were co-transfected with 1-10-50 or 250 ng of each of the miSNCA constructs and Dual Luc reporter carrying SNCA gene. From 17 miSNCA constructs (SEQ ID NO.71-77 and SEQ ID NO. 82-91) designed to target SNCA gene, miSNCA2 (SEQ ID NO. 71), miSNCA5 (SEQ ID NO. 72), miSNCA7 (SEQ ID NO. 73), miSNCA12 (SEQ ID NO. 74), miSNCA13 (SEQ ID NO. 75), miSNCA15 (SEQ ID NO. 76) and miSNCA16 (SEQ ID NO. 77) induced the most potent dose-dependent decrease in the RL/FL ratio (FIG. 3). To further determine the potency, above mentioned constructs were further used for titration experiments. The constructs were co-transfected into HEK293T cells in different concentrations; 0.1, 1, 10, or 100 ng with 10 ng of SNCA luciferase reporter plasmid. According to these results, the transfections at 100 ng of miSNCA plasmids showed at least 50% decrease for all the miSNCA candidates used in the titration experiments (FIG. 5). For further testing in different models, miSNCA5, miSNCA13 and miSNCA15 were chosen because of their relatively higher efficacy in decreasing mSNCA levels.

### Example 2. Lowering of Endogenous SNCA Expression in Transfected Cells with the Scaffold 1 and Scaffold 2 Constructs

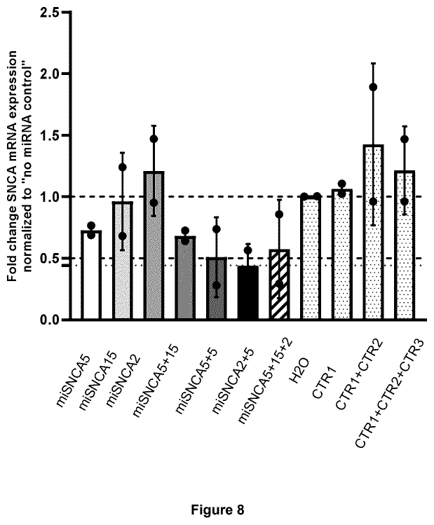

The miSNCA5, miSNCA15 and miSNCA2 Scaffold 1 (SEQ ID NO. 96, 98, 95) and Scaffold 2 constructs (miSNCA2+5, miSNCA5+15, miSNCA5+5, miSNCA5+15+2; SEQ ID Nos. 78 to 81, respectively) and control miRNAs for Scaffolds 1 and 2 (SEQ ID NO. 93, 94, and 99) single (CTR1), double (CTR1+CTR2) or triple scrambled miRNAs (CTR1+CTR2+CTR3)) were chosen for testing the knockdown of SNCA mRNA expression in cells (FIGS. 7A and B). The knockdown of the endogenous SNCA gene expression in HEK293T cells was confirmed by RT-QPCR on transfected cells. Transfection of 50 ng of miRNA plasmid resulted in a decrease of SNCA mRNA expression of 60-40% by all miSNCA Scaffold 2 candidates tested (FIG. 8). A similar decrease in protein expression is expected.

### Example 3. Processing of miSNCA Constructs Upon Transfection in Cells (NGS Data)

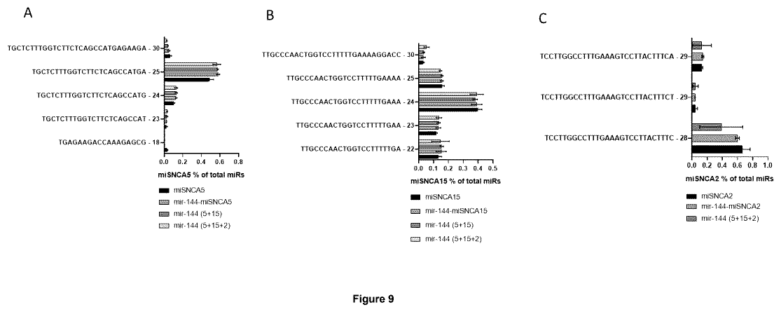

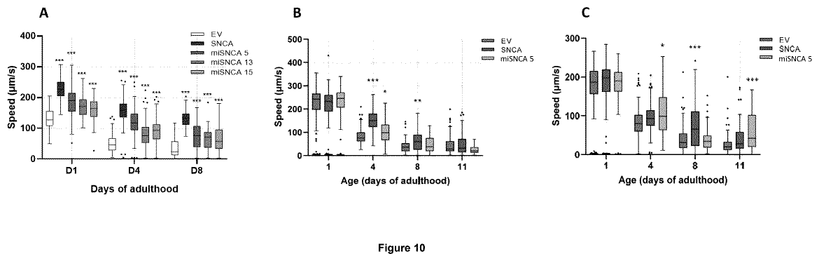

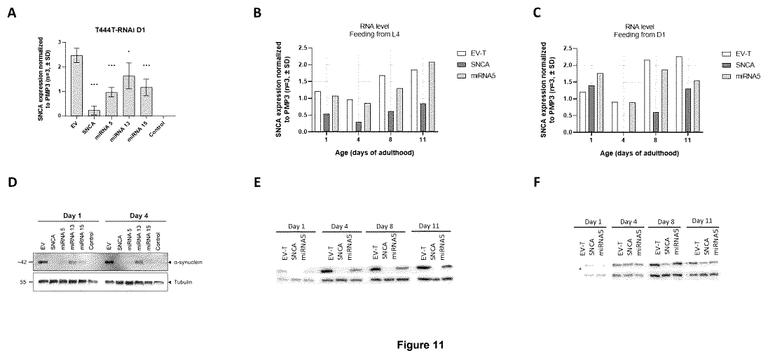

The processing of miRNAs was investigated by alignment of the reads to the pre-miRNA sequences of miSNCA5, miSNCA15 and miSNCA2 constructs in Scaffold 1 and Scaffold 2 backbones. According to the results, all miSNCAs were processed the same way in both Scaffold 1 or Scaffold 2 backbones. The length of the most abundant form for miSNCA5 was 25 nts, followed by 24 nts (FIG. 9A), for miSNCA15 was 24 nts, followed by 25 nts (FIG. 9B), for miSNCA2 was 28 nts (FIG. 9C)

### Example 4. AAV-miSNCA Transduction and Dose-Dependent SNCA mRNA Lowering in Human Cells

To investigate the ability of AAV5 or AAV6-Scaffold 1 or Scaffold 2 to transduce and deliver the packaged expression cassette, DA neurons or forebrain neurons and/or LUHMES-derived DA neurons are transduced at various Multiplicity of Infections (MOI); 10⁴, 10⁵, 10⁶ and 10⁷. The vector DNA levels are measured; a dose-dependent vDNA level increase in these cells is expected. RNA is isolated from the transduced cells and mRNA levels of SNCA is measured using RT-qPCR assays, with a dose dependent mRNA decrease of SNCA levels in these transduced cells being expected.

### Example 5. AAV5-miSNCA and AAV6-miSNCA Transduction in In Vitro and In Vivo Studies: Evaluation of miSNCA Processing

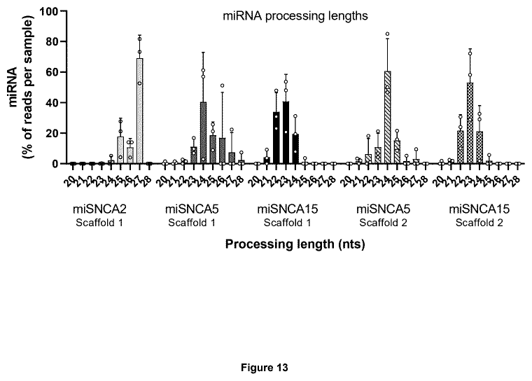

To evaluate the miRNA processing of the miSNCA2, miSNCA5 and miSNCA15 in Scaffold 1 and Scaffold 2, AAV5-miSNCA2, AAV5-miSNCA5, AAV5-miSNCA15, AAV5-miSNCA5+15 were used to transduce human induced-pluripotent stem cell (iPSC)-derived forebrain neurons at MOI5. Total RNA was isolated from these samples and small RNA sequencing was performed. The processing of miRNAs was investigated by alignment of the reads to the pre-miRNA sequences of miSNCA2, miSNCA5 and miSNCA15 constructs in Scaffold 1 and Scaffold 2 backbones. According to the results, all miSNCAs were processed the same way in both Scaffold 1 or Scaffold 2 backbones (FIG. 13). The length of the most abundant form for miSNCA2 was 27 nts, followed by 25-26 nts, for miSNCA5 was 24 nts, followed by 25-24 nts, for miSNCA15 was 23 nts followed by 22-24 nts (FIG. 13).

### Example 6. AAV5-miSNCA Processing from In Vivo Samples

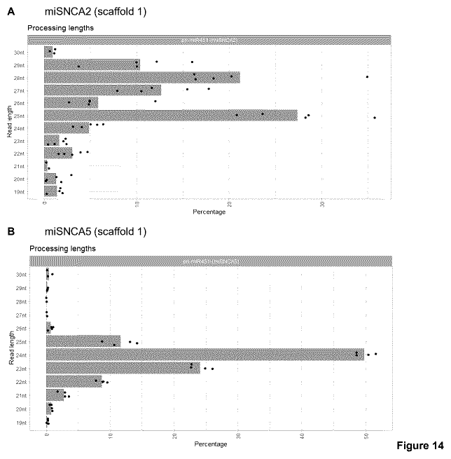

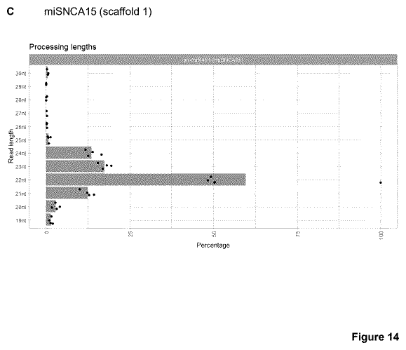

To investigate the miRNA processing and isoforms of the miRNA expressed from Scaffold 1, RNA isolated from striatum tissue samples from in vivo Study 2 were analysed by small RNA sequencing (Next Generation Sequencing, by GenomeScan). The processing of the miSNCA2, miSNCA5 and miSNCA15 in Scaffold 1 in vivo samples was similar to the in vitro processing of these miRNAs (FIG. 13 versus FIG. 14). The length of the most abundant isoforms; for miSNCA2 were 25 and 28 nts followed by 27 and 29 nts (FIG. 14A), for miSNCA5 were 24 nts, followed by 23, 25 and 22 nts (FIG. 14B), and for miSNCA15 were 22 nts followed by 23, 21 and 24 nts (FIG. 14C).

### In Vivo Studies

### Example 7. AAV5-miSNCA Candidates Lowered Human SNCA mRNA Expression in α-Syn KI Rats (Study 1)

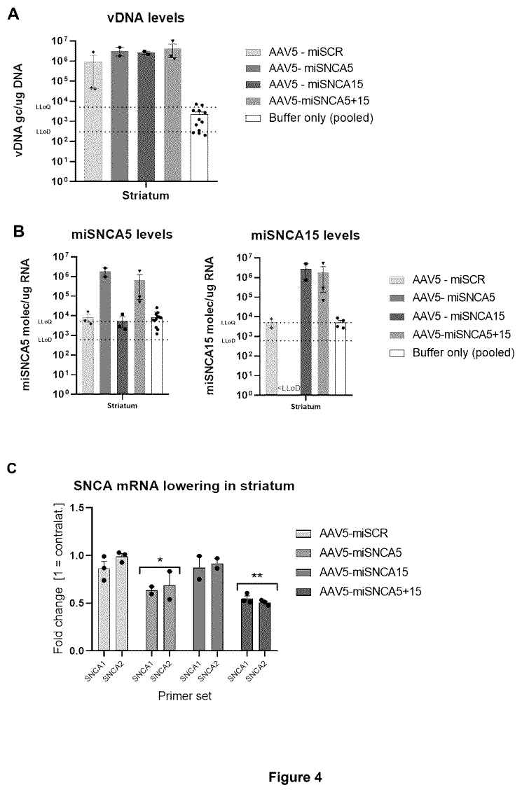

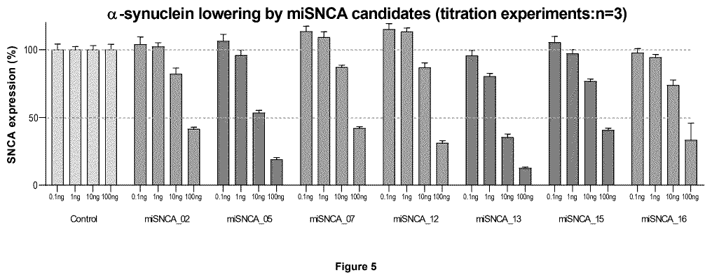

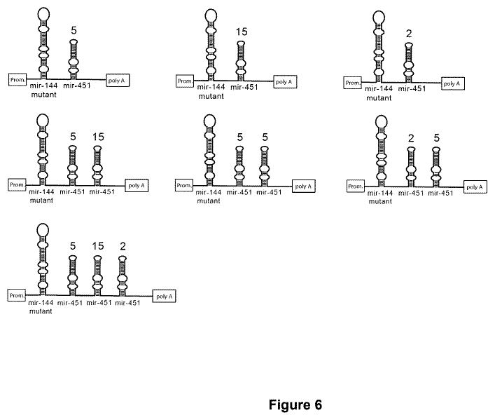

To evaluate the capacity of two of the designed candidates (miSNCA5 and miSNCA15) in lowering human SNCA mRNA expression, AAV5-miSCR (miSCR non-targeting scramble control (SEQ ID NO. 100), AAV5-miSNCA5 or AAV5-miSNCA15 were injected into the left striatum of adult α-syn KI rats. The miSNCA13 was excluded from in vivo studies because it was targeting a region outside of the humanized part of SNCA KI rat model, and it has 3 mismatches to the wt rat SNCA gene. As an additional control, formulation buffer was injected in the right striatum. At the single dose used, vDNA was detected in the AAV5 injected brain hemispheres, while in the control hemispheres, vDNA levels were below the lower limit of quantification (LLOQ) (FIG. 4A). Transduction led to expression of miSNCA candidates 5 and 15 in a vector-specific manner (FIG. 4B): miSNCA5 was detected only in the AAV5-miSNCA5 injected hemisphere, and miSNCA15 only in the AAV5-miSNCA15 injected one. At the single dose used, AAV5-miSNCA5 was effective in reducing SNCA mRNA expression in the injected striatum, as evaluated by two different RT-QPCR SNCA assays (primer set SNCA1 and primer set SNCA2), compared to the control striatum (FIG. 4C). This study shows AAV5-miSNCA candidates for the treatment of Parkinson's disease can reduce expression of human SNCA mRNA, and eventually reduce associated toxicity of α-synuclein. Moreover, combination of miSNCA5 and miSNCA15 is more efficacious in lowering the mRNA levels of SNCA.

### Example 8. Proof of Concept Study in AAV-Syn Rat Model (Study 2)

Different AAV5-miSNCA Scaffold 1 and AAV5-miSNCA Scaffold 2 candidates were tested in the AAV-Syn rat model. The AAV5-miSNCA vectors recovered the disease phenotype in the AAV-Syn rat model, in motor phenotype, molecular and neurochemical alterations.

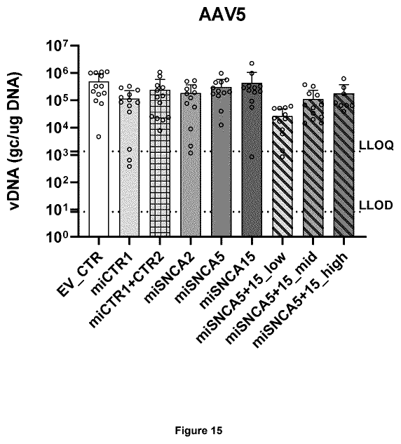

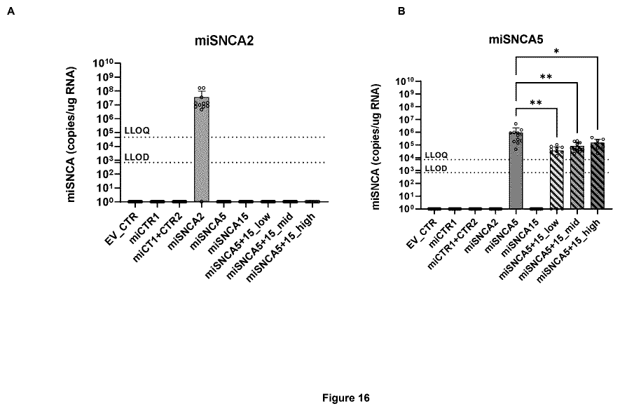

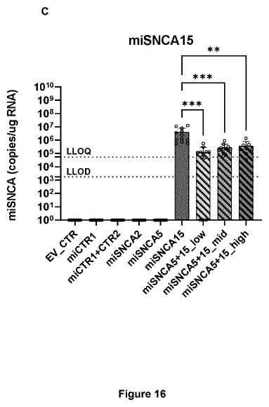

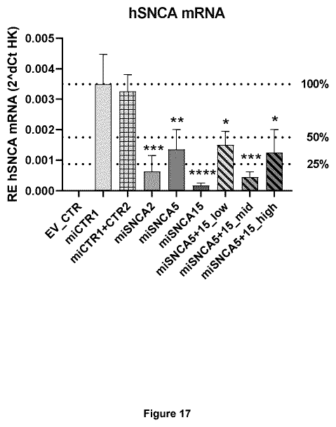

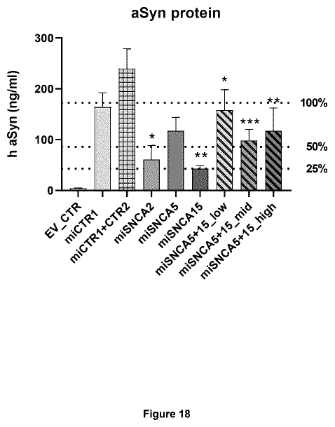

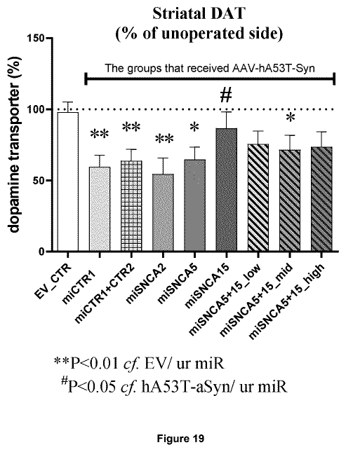

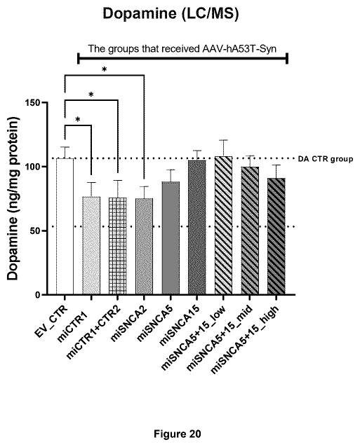

To determine the in vivo proof of concept of lowering SNCA levels, and the relation to motor phenotype improvement, a rat PD model was used (AAV1/2-hA53T-aSyn; viral-induced overexpression of human A53T α-syn). The right substantia nigra (SN) received unilateral injections of the disease-inducing AAV1/2-hSNCA vector, followed by administration of the different therapeutic vectors (AAV5-miSNCA) and respective controls miCTR1 or miCTR1+CTR2 Empty vector (EV_CTR) was used as control for the disease model. In all the injected groups, at the single dose used, vDNA was detected in the striatum of the AAV5 injected brain hemispheres. In the last three AAV5-miSNCA Scaffold 2 groups with low-mid and high doses, dose dependent vDNA levels were detected (FIG. 15), while in the control hemispheres (left striatum), vDNA levels were below the lower limit of quantification (LLOQ) (not shown). Transduction led to specific expression of miSNCA2, miSNCA5 or miSNCA15, in a vector-specific manner (FIG. 16 A, B, and C): miSNCA2 was detected only in the AAV5-miSNCA2 injected group, miSNCA5 was detected only in the AAV5-miSNCA5 and miSNCA5+15 injected groups, miSNCA15 only in the AAV5-miSNCA15 and miSNCA5+15 injected groups, and none was detected in the other samples from the negative control groups. In A53T-aSyn animals, all of the AAV5-miSNCA candidates were effective in reducing SNCA mRNA expression in the striatum of the injected hemisphere, as evaluated by Taqman RT-qPCR assays (SNCA2 primer and probe combination); AAV5-miSNCA scaffold 1 candidates were compared to the control striatum injected with unrelated miRNA (light grey solid bars; miCTR1) and AAV5-miSNCA scaffold 2 candidates were compared to the control striatum injected with unrelated miRNA (light grey checkered bars; miCTR1+CTR2) (FIG. 17). The miSNCA expression also led to lowering at the protein level reflected by reduced α-syn protein levels in all AAV5-miSNCA treated groups with respect to controls, as measured by ELISA (FIG. 18). Dopamine transporter deficits measured by [125I]-RTI-121 autoradiography were apparent in the A53T-aSyn animals injected with a control (non-targeting) miRNA (unrelated miR, groups 2 and 3 (miCTR1 and miCTR1+CTR2 in Table 6), similar to the deficits observed in PD patients, and were corrected in the miSNCA15 and miSNCA5+15 (low and high dose) treated groups (groups 6, 7 and 9 in Table 6) (FIG. 19). Related to the metabolite changes in this model, correction of hA53T-aSyn-induced striatal dopamine deficits by miSNCA candidates were observed in groups 5-9. FIG. 20 shows the dopamine levels in the study groups.

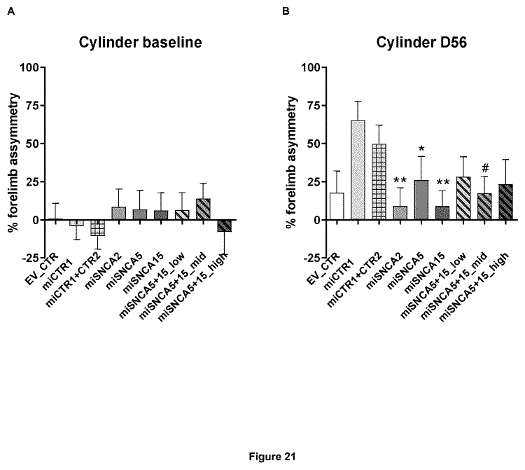

The motor behavioral deficits measured by percent asymmetry of left paw use were significantly rescued in the groups which received miSNCA2, miSNCA5, miSNCA15 or miSNCA5+15 (mid dose) (groups 4, 5, 6 and 8) treatment on day 56 compared to the baseline levels (FIG. 21 A and B).

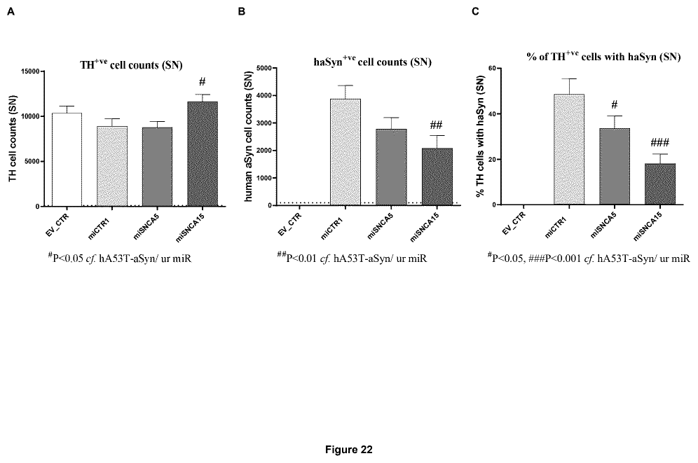

The molecular, biochemical and motor behavioral results were supported with histological observations. Immunostaining and quantification of dopaminergic (TH positive) and α-syn positive neurons in the substantia nigra showed that both AAV5-miSNCA5 and AAV5-miSNCA15 candidates rescued dopaminergic (TH) neuron cell loss (FIG. 22A) and reduced human α-syn positive cell counts (FIG. 22B). This was reflected by a reduction of the percentage of positive dopaminergic (TH) cells that expressed α-syn (FIG. 22C).

Overall, AAV5-miSNCA rescued the disease phenotype in the AAV-Syn rat model, improved motor phenotype and rescued the molecular and neurochemical alterations.

### Example 9. Phenotypic Rescue of Motor Phenotype in C. Elegans PD Model (Study 3)

In order to compare the speed of movement between the C. elegans worms (expressing human SNCA gene in their body wall muscle causing decreased motility) that were fed by different plasmids-expressing E. coli, 100 worms per condition were video tracked. According to the results the worms that were fed by full-length SNCA RNAi-expressing or miSNCA-expressing E. coli showed an improvement in their speed of movement compared to worms that were fed by the empty plasmid transformed E. coli. The worms that were treated with full-length SNCA or miSNCAs had an increased speed compared to the untreated disease model worms on all days that their movements were tracked. These results show that decreasing the expression of SNCA gene, thereby reducing the α-synuclein levels, is improving the motor phenotype in this C. elegans PD model.

RNAi by using miSNCA candidates has been shown to be effective when treated at different stages of the C. elegans development; Larval stage 1 (L1), Larval Stage 4 (L4) and adult stage day 1 (Day1). C. elegans motor behavior was rescued with treatments at, L1, L4 and Day1 stages (FIG. 10 A-C). The SNCA mRNA levels (FIG. 11 A-C) and α-synuclein protein levels (FIG. 11 D-F) were also shown to be decreased at these conditions.

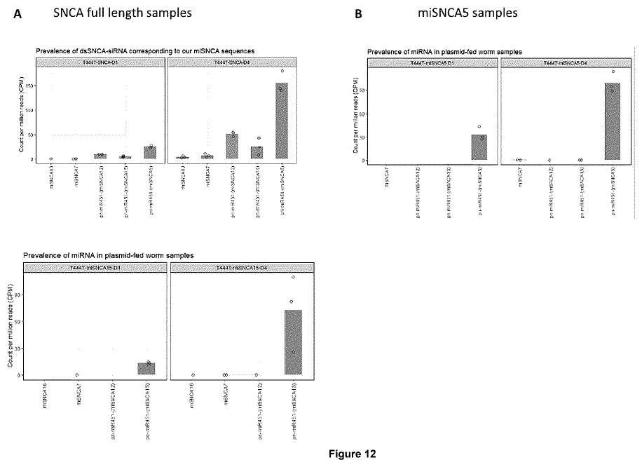

RNAi by full-length SNCA and miSNCAs rescued the motor phenotype behaviour caused by SNCA overexpression in the muscle cells of C. elegans PD model. SNCA full-length RNAi always shows a much more efficacious lowering of the SNCA mRNA and α-synuclein protein lowering. This could be explained by SNCA full-length RNAi resulting in different pieces of small RNAs that are collectively much more effective in lowering the mRNA expression of SNCA. Additionally, this could be due to increased dosage of the miRNAs introduced by full-length SNCA RNAi. The small RNA sequencing results demonstrated this by showing many different SNCA fragments (majority of the fragment size ranges between 20-25 bps). Some of the fragments had overlapping sequences with the miSNCAs designed within this study (FIG. 12), supporting the beneficial effect of combining different miRNAs to increase potency.

### Example 10. Proof of Concept Study in PFF Rat Model (Study 4)

Different AAV-miSNCA candidates are tested in the PFF rat model. A decrease in the pathological spreading of α-syn PFF in the rat brain is expected.

### Example 11. Dose-Response Study in Rodent Model of Parkinson's Disease (Study 5)

Several doses of the most potent AAV5-miSNCA Scaffold 1 and Scaffold 2 candidates are tested in a relevant rodent (rat or mouse) Parkinson's disease model (transgenic line). It is expected that AAV5-miSNCA Scaffold 1 and Scaffold 2 rescues the phenotype of molecular, histological and motor readouts.

1. A nucleic acid comprising a sequence encoding a first RNA and a sequence encoding a second RNA, wherein:
i. the first and second RNA each comprise a hairpin;
ii. the first RNA comprises a first guide sequence of at least 19 nucleotides substantially complementary to part of an alpha-synuclein (SNCA) gene;
iii. the second RNA comprises a second guide sequence of at least 19 nucleotides substantially complementary to part of an SNCA gene.

2. The nucleic acid according to claim 1, wherein, in a 5′ to 3′ direction, the sequence encoding the first RNA is followed by a first spacer comprising at least 15 nucleotides, which first spacer is followed by the sequence encoding the second RNA.

3. The nucleic acid according to claim 1, wherein the first RNA comprises a sequence selected from the group consisting of: SEQ ID NO. 11; SEQ ID NO. 101; and variants thereof; and the second RNA comprises SEQ ID NO. 11 or a variant thereof.

4. The nucleic acid according to claim 1, wherein the first RNA and the second RNA each comprise SEQ ID NO. 11 or a variant thereof.

5. The nucleic acid according to claim 4, further comprising a sequence encoding a third RNA, and wherein the third RNA comprises SEQ ID NO. 34 or a variant thereof.

6. The nucleic acid according to claim 5, wherein, in a 5′ to 3′ direction, the sequence encoding the third RNA is followed by a second spacer comprising at least 15 nucleotides, which second spacer is followed by the sequence encoding the first RNA, followed by the first spacer and the sequence encoding the second RNA.

7. The nucleic acid according to claim 5, wherein the third RNA is mutated to reduce or eliminate miR144 processing and/or expression of the third RNA.

8. The nucleic acid according to claim 1, wherein each of the guide sequences is substantially complementary to a sequence selected from the group consisting of: SEQ ID NO. 4 to SEQ ID NO. 10.

9. An expression cassette comprising a nucleic acid, wherein the expression cassette is a DNA molecule, and wherein the nucleic acid comprises a sequence encoding a first RNA and a sequence encoding a second RNA, wherein:
i. the first and second RNA each comprise a hairpin;
ii. the first RNA comprises a first guide sequence of at least 19 nucleotides substantially complementary to part of an alpha-synuclein (SNCA) gene; and
iii. the second RNA comprises a second guide sequence of at least 19 nucleotides substantially complementary to part of an SNCA gene.

10. The expression cassette according to claim 9, wherein the nucleic acid is operably linked to a promoter and optionally to a poly-A signal.

11. The expression cassette according to claim 9, wherein the expression cassette is flanked by at least one AAV Inverted Terminal Repeat (ITR).

12. An adeno-associated virus (AAV) vector comprising an expression cassette, wherein the expression cassette comprises a nucleic acid, wherein the expression cassette is a DNA molecule, and wherein the nucleic acid comprises a sequence encoding a first RNA and a sequence encoding a second RNA, wherein:
i. the first and second RNA each comprise a hairpin;
ii. the first RNA comprises a first guide sequence of at least 19 nucleotides substantially complementary to part of an alpha-synuclein (SNCA) gene; and
iii. the second RNA comprises a second guide sequence of at least 19 nucleotides substantially complementary to part of an SNCA gene.

13. The AAV vector according to claim 12 comprising an AAV5 or AAV9 capsid protein.

14. A pharmaceutical composition comprising:
a nucleic acid comprising a sequence encoding a first RNA and a sequence encoding a second RNA, wherein:
i. the first and second RNA each comprise a hairpin;
ii. the first RNA comprises a first guide sequence of at least 19 nucleotides substantially complementary to part of an alpha-synuclein (SNCA) gene;
iii. the second RNA comprises a second guide sequence of at least 19 nucleotides substantially complementary to part of an SNCA gene;
an expression cassette comprising the nucleic acid, wherein the expression cassette is a DNA molecule, or an adeno-associated virus (AAV) vector comprising the expression cassette; and
at least one pharmaceutically acceptable excipient.

15.-20. (canceled)
